(19) United States
(12) Patent Application Publication
(10) Pub. No.: US 2026/0308266 A1
(43) Pub. Date: Oct. 8, 2026

(54) Novel Use of Anti-BLyS Antibodies

(71) Applicants: GlaxoSmithKline Intellectual Property Management Limited, Stevenage (GB); GlaxoSmithKline Intellectual Property Limited, Stevenage (GB)

(72) Inventors: Elaine IRVING, Stevenage (GB); Mary MORSE, Stevenage (GB); Svetlana NIHTYANOVA, London (GB)

(73) Assignees: GlaxoSmithKline Intellectual Property Management Limited, Stevenage (GB); GlaxoSmithKline Intellectual Property Limited, Stevenage (GB)

(21) Appl. No.: 19/484,445

(22) PCT Filed: May 17, 2024
(86) PCT No.: PCT/EP2024/063695

§ 371 Date: Nov. 14, 2025

Related U.S. Application Data
(60) Provisional application No. 63/503,013, filed on May 18, 2023.

(51) Int. Cl. C07K 16/28; A61K 9/00; A61K 38/00; A61K 45/06; A61P 11/00

(52) U.S. Cl. CPC C07K 16/2875; A61K 9/0019; A61K 38/00; A61K 45/06; A61P 11/00; C07K 2317/565; C07K 2317/73; C07K 2317/76

(57) ABSTRACT

The present disclosure provides anti-BlyS antibodies for use in the treatment of connective tissue disease, in particular, connective tissue disease-associated interstitial lung disease, including systemic sclerosis, in particular, systemic sclerosis associated interstitial lung disease, for example, diffuse cutaneous systemic sclerosis-associated interstitial lung disease. Also provided is a method for the treatment of connective tissue disease-associated interstitial lung disease, including systemic sclerosis associated-interstitial lung disease comprising administering to a subject in need thereof a therapeutically effective amount of an anti-BlyS antibody.

### SEQUENCE LISTING SUBMITTED ELECTRONICALLY

This application contains a sequence listing, which is provided in XML format with a file name “70336WO01.xml”. The XML file has a size of about 14 kilobytes and was created on or about Apr. 23, 2024. The sequence listing submitted electronically is part of the specification and is incorporated herein by reference in its entirety.

### FIELD OF THE INVENTION

The present invention relates to anti-BlyS antibodies for use in the treatment of Connective Tissue Disease (CTD) such as Connective Tissue disease associated Interstitial Lung disease (CTD-ILD), Systemic sclerosis (SSc) and Systemic sclerosis associated interstitial lung disease (SSc-ILD). The present invention also relates to a method for the treatment of CTD-ILD or SSc-ILD and to a method of improving the quality of life of a human subject suffering from CTD-ILD or SSc-ILD using an anti-BlyS antibody.

### BACKGROUND

Connective tissue diseases (CTDs) refer to a group of autoimmune disorders which comprise diseases such as systemic sclerosis (SSc), rheumatoid arthritis (RA), primary Sjögren's syndrome (pSS), idiopathic inflammatory myositis (IIM), such as polymyositis (PM), dermatomyositis (DM) and anti-synthetase syndrome, systemic lupus erythematous (SLE), and mixed connective tissue disease (MCTD) which, while distinct, share common pathological features including autoimmunity and immune-mediated organ dysfunction.

Systemic sclerosis (SSc) is a rare heterogeneous chronic autoimmune disease characterised by widespread fibrosis, or the excessive production and accumulation of collagen and other extracellular matrix proteins, leading to thickening and hardening of affected tissues. This fibrosis can lead to impaired organ function, and potential organ failure, hence the high morbidity and mortality associated with the condition. SSc is divided into two major subsets determined by the extent of skin affected: limited cutaneous systemic sclerosis (lcSSc) and diffuse cutaneous systemic sclerosis (dcSSc).

LcSSc is characterised by fibrosis of the skin distal to the elbows and knees and can involve the face and neck. In contrast, dcSSc is associated with more diffuse fibrosis of the skin, with proximal limb or trunk involvement, as well as a higher risk of internal organ complications such as cardiac scleroderma, scleroderma renal crisis, and lung fibrosis (Khanna, et al., (2022) Arthritis Rheumatol. 2022; 74(1):13-27).

Interstitial lung disease (ILD) is a condition characterised by non-infective inflammation and/or fibrosis affecting the lung parenchyma, in which scarring to the lungs leads to reduction of lung function, and is a common systemic disease manifestation of SSc but also connective tissue disease (CTD) more broadly. It has been reported that the proportion of each CTD subtype with ILD is as follows: MCTD, 56%; SSc, 47%; IIM, 41%; pSS 17%, RA, 11%; and SLE, 6% (Joy G M, et al. Eur Respir Rev. 2023 Mar. 8; 32(167):220210).

Systemic sclerosis associated ILD (SSc-ILD) is currently the leading cause of death related to systemic sclerosis. Approximately one third of lcSSc subjects develop ILD, while more than half of subjects with dcSSc can develop ILD, with some cohorts reporting up to 90% of dcSSc subjects having some degree of fibrotic change when imaged with high-resolution computed tomography (DeSantis, et al., (2005) Respir Res. 2005; 6(1):96; and Frantz, et al., (2020) Autoimmun Rev. 2020; 19(2):102452. doi: 10.1016/j.autrev.2019.102452).

Despite recent approvals of therapeutics, including Nintedanib and Tocilizumab, for some subjects with ILD associated with Systemic sclerosis, there remains a significant unmet need for well tolerated targeted therapies for patients with CTD-associated ILD (CTD-ILD), which offer CTD-ILD subjects, including those with SSc-ILD, benefit beyond stabilisation of lung function.

B lymphocyte stimulating factor (BlyS/BAFF) is a cytokine that plays a crucial role in the survival and proliferation of B cells. The dysregulation of BlyS has been implicated in the pathogenesis of several autoimmune diseases, such as systemic lupus erythematosus (SLE), rheumatoid arthritis, and Sjögren's syndrome. In these conditions, elevated levels of BlyS lead to the survival and expansion of autoreactive B cells, which can produce autoreactive antibodies and contribute to disease progression.

Despite some early work suggesting the use of belimumab (an anti-BlyS/anti-BAFF antibody) in the treatment of Systemic sclerosis the studies were inconclusive. An example of this work is the 2017 pilot study which investigated the use of belimumab in combination with mycophenolate mofetil in the treatment of early diffuse systemic sclerosis (Gordon et al., (2018) Arthritis Rheumatol. 2018; 70(2):308-16. doi: 10.1002/art.40358). The study did not specifically select for subjects with ILD at baseline and was unable to determine a significant improvement on skin thickening or lung function following treatment with belimumab compared to placebo due to lack of statistical power. Another such study investigated the use of belimumab in the treatment of a single subject suffering with systemic lupus erythematosus (SLE) and scleroderma overlap syndrome (Mwangi, et al., (2021) Cureus. 2021; 13(11):e19218. doi:10.7759/cureus.19218). Though it appeared that the subject showed improvement in lung capacity and other pulmonary function tests, SLE subjects also suffer from ILD and therefore direct conclusion to a subject with only SSc or a documented primary diagnosis of SSc could not be made.

There remains a need to identify therapies for CTD and CTD-ILD, including Systemic sclerosis and Systemic sclerosis associated ILD. In particular, to identify therapies that have an impact on lung function as well as on extra-pulmonary disease manifestations, including skin thickening in subjects with SSc, and general symptoms, such as fatigue, that impact quality of life (QoL) of those suffering with the active disease and to identify those subjects most likely to respond to treatment.

### SUMMARY

In one aspect, there is provided an anti-BlyS antibody for use in the treatment of connective tissue disease-associated interstitial lung disease in a human subject in need thereof, wherein the connective tissue disease-associated interstitial lung disease comprises systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease, and wherein the connective tissue disease-associated interstitial lung disease does not comprise systemic lupus erythematosus-associated interstitial lung disease.

In one aspect, there is provided a method for the treatment of connective tissue disease-associated interstitial lung disease in a human subject in need thereof comprising administering to the human subject a therapeutically effective amount of the anti-BlyS antibody according to the invention, or a method for the treatment of systemic sclerosis-associated interstitial lung disease in a human subject in need thereof comprising administering to the human subject a therapeutically effective amount of the anti-BlyS antibody according to the invention.

In one aspect, there is provided a method of improving the quality of life of a human subject suffering from connective tissue disease-associated interstitial lung disease, wherein the human subject is administered a therapeutically suitable amount of the anti-BlyS antibody according to the invention, or a method of improving the quality of life of a subject suffering from systemic sclerosis-associated interstitial lung disease, wherein the human subject is administered a therapeutically suitable amount of the anti-BlyS antibody according to the invention.

In one aspect, there is provided a BlyS antagonist for use in the treatment of Connective Tissue disease associated Interstitial Lung disease (CTD-ILD).

In one aspect, there is provided a BlyS antagonist for use in increasing progression free survival of a human subject with Systemic sclerosis.

In one aspect, there is provided a BlyS antagonist for use in the treatment of Systemic sclerosis (SSc), for example, in one aspect, there is provided a BlyS antagonist for use in the treatment of Systemic sclerosis-associated interstitial lung disease (SSc-ILD).

In a further aspect, there is provided a BlyS antagonist for use in the treatment of diffuse cutaneous systemic sclerosis (dcSSc), for example, in the treatment of diffuse cutaneous systemic sclerosis-associated interstitial lung disease (dcSSc-ILD). In a specific aspect, there is provided a BlyS antagonist for use in the treatment of early dcSSc-ILD. In a further aspect, there is provided a BlyS antagonist for use in the treatment of limited cutaneous systemic sclerosis (lcSSc), for example, in the treatment of limited cutaneous systemic sclerosis-associated interstitial lung disease (lcSSc-ILD). In a specific aspect, there is provided a BlyS antagonist for use in the treatment of early lcSSc-ILD. In one aspect, there is provided a BlyS antagonist for use in the reduction of disease progression in a human subject with Systemic sclerosis, for example, with SSc-ILD.

In one aspect, there is provided a BlyS antagonist for use in the reduction of skin thickening in the skin of a human subject with Systemic sclerosis, for example, with SSc-ILD.

In one aspect, there is provided a BlyS antagonist for use in the improvement in quality of life of a human subject, suffering from Systemic sclerosis, for example, with SSc-ILD.

In one aspect, there is provided a BlyS antagonist for use in the treatment of SSc in a human subject, wherein the human subject is anti-topoisomerase (anti-Scl70) autoantibody positive.

In a further aspect, there is provided a BlyS antagonist for use in the treatment of SSc-ILD in a human subject, wherein the human subject is characterised by:
(i) Presence of thickened skin with a modified Rodnan Skin Score ≥10 points, optionally, ≥15 points;
(ii) Disease duration ≤7 years; and
(iii) Anti-topoisomerase I (anti-Scl70) autoantibody positive.

In a further aspect, there is provided a BlyS antagonist for use in the treatment of SSc-ILD in a human subject, wherein the human subject is characterised by:
(i) Presence of thickened skin with a modified Rodnan Skin Score ≥10 points, optionally, ≥15 points;
(ii) Disease duration ≤7 years; and
(iii) Anti-RNA polymerase autoantibody positive.

In a further aspect, there is provided a BlyS antagonist for use in the treatment of SSc-ILD in a human subject, wherein the human subject is characterised by:
(i) Presence of thickened skin with a modified Rodnan Skin Score ≥10 points, optionally, ≥15 points;
(ii) Disease duration ≤7 years; and
(iii) Anti-centromere polymerase autoantibody negative.

In one aspect, there is provided a method of treating Systemic sclerosis in a human subject comprising administering an anti-BlyS antagonist.

In one aspect, there is provided a BlyS antagonist for use in the manufacture of a medicament for the treatment of Systemic sclerosis.

In one aspect, there is provided a pharmaceutical composition comprising an anti-BlyS antagonist for use in the treatment of Systemic sclerosis.

Further aspects of the present disclosure are provided throughout the detailed description, below.

### DESCRIPTION OF FIGURES

FIG. 1. BLyS gene expression in B cell subsets.

FIG. 2. Expression of B cells gene subsets is increased in early dcSSc.

FIGS. 3A and 3B. Positive correlation of B cell gene sets and disease measures in dcSSc skin.

FIGS. 4A and 4B. Belimumab treatment reduced expression of genes associated with activated B cells in SSc skin.

FIG. 5. B cell gene sets and disease measures in lung.

### DETAILED DESCRIPTION

In one aspect, there is provided an anti-BlyS antibody for use in the treatment of connective tissue disease-associated interstitial lung disease in a human subject in need thereof, wherein the connective tissue disease-associated interstitial lung disease comprises systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease, and wherein the connective tissue disease-associated interstitial lung disease does not comprise systemic lupus erythematosus-associated interstitial lung disease.

In one embodiment, the interstitial lung disease does not comprise usual interstitial pneumonia. In another embodiment, the interstitial lung disease comprises usual interstitial pneumonia. In one embodiment, the interstitial lung disease comprises nonspecific interstitial pneumonia, organising pneumonia, and/or lymphocytic interstitial pneumonia.

In one embodiment, the connective tissue disease-associated interstitial lung disease is rheumatoid arthritis-associated interstitial lung disease, and the interstitial lung disease comprises usual interstitial pneumonia. In one embodiment, the connective tissue disease-associated interstitial lung disease is systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease, and the interstitial lung disease comprises nonspecific interstitial pneumonia. In one embodiment, the connective tissue disease-associated interstitial lung disease is Sjögren's syndrome-associated interstitial lung disease, and the interstitial lung disease comprises lymphocytic interstitial pneumonia. In one embodiment, the connective tissue disease-associated interstitial lung disease is an idiopathic inflammatory myopathy-associated interstitial lung disease, and the interstitial lung disease comprises organising pneumonia. In some embodiments, the idiopathic inflammatory myopathy comprises polymyositis, dermatomyositis, or anti-synthetase syndrome.

In one embodiment, the treatment is characterised by a reduction in a disease progression. In one embodiment, the disease progression is connective tissue disease progression and/or interstitial lung disease progression. In one embodiment, the disease progression is interstitial lung disease progression, and the interstitial lung disease progression is characterised by a reduction in forced vital capacity. In one embodiment, the interstitial lung disease progression is further characterised by: (i) a reduction in forced vital capacity of ≥5% relative to baseline, optionally, a reduction in forced vital capacity of ≥10% relative to baseline; or (ii) a reduction in forced vital capacity of ≥5% to <10% relative to baseline and a reduction in diffusing capacity of the lung for carbon monoxide of ≥15% relative to baseline. In another embodiment, the interstitial lung disease progression is characterised by two or more of the following (a) to (c) occurring within a 12 month time period: (a) a worsening of respiratory symptoms; (b) a physiological evidence of interstitial lung disease progression, wherein the physiological evidence of interstitial lung disease progression comprises: (i) an absolute reduction in forced vital capacity of ≥55% predicted; and/or (ii) an absolute reduction in diffusing capacity of the lung for carbon monoxide of ≥10% predicted; and/or (c) a radiological evidence of interstitial lung disease progression.

In one embodiment, the treatment is characterised by: (a) an improvement in one or more measures relative to baseline in a human subject, wherein the one or more measures are selected from: (i) forced vital capacity; (ii) diffusing capacity of the lung for carbon monoxide; (iii) connective tissue disease-associated interstitial lung disease symptom severity and/or (iv) quality of life, and/or (b) a reduction in one or more measures relative to baseline in the human subject, wherein the one or more measures are selected from: (iv) extent of interstitial lung disease; and/or (v) corticosteroid use. In one embodiment, the improvement in connective tissue disease-associated interstitial lung disease symptom severity comprises a reduction in fatigue, a reduction in dyspnea, and/or a reduction in cough relative to baseline.

In one embodiment, the anti-BlyS antibody according to the invention is for use in the treatment of systemic sclerosis-associated interstitial lung disease in the human subject. In one embodiment, the systemic sclerosis-associated interstitial lung disease is diffuse cutaneous systemic sclerosis-associated interstitial lung disease. In one embodiment, the diffuse cutaneous systemic sclerosis-associated interstitial lung disease is early diffuse cutaneous systemic sclerosis-associated interstitial lung disease. In another embodiment, the systemic sclerosis-associated interstitial lung disease is limited cutaneous systemic sclerosis-associated interstitial lung disease. In one embodiment, the limited cutaneous systemic sclerosis-associated interstitial lung disease is early limited cutaneous systemic sclerosis-associated interstitial lung disease.

In one embodiment, treatment is characterised by a reduction in disease progression. In one embodiment, the disease progression is characterised by a reduction in forced vital capacity. In one embodiment, the disease progression is further characterised by: (i) a reduction in forced vital capacity of ≥5% relative to baseline, optionally, a reduction in forced vital capacity of ≥10% relative to baseline; or (ii) a reduction in forced vital capacity of ≥5% to <10% relative to baseline and a reduction in diffusing capacity of the lung for carbon monoxide of ≥15% relative to baseline. In one embodiment, the disease progression is further characterised by an increase in modified Rodnan skin score of ≥20% relative to baseline. In one embodiment, the treatment is characterised by: (a) an improvement in forced vital capacity relative to baseline; and/or (b) a reduction in modified Rodnan skin score of ≥20% relative to baseline.

In one embodiment, a reduction in one or more measures relative to baseline is achieved in the human subject, wherein the one or more measures are selected from: (i) Fatigue, optionally, wherein the fatigue is as assessed by the functional assessment of chronic illness therapy-fatigue score compared to baseline; (ii) Health Assessment Questionnaire Disability Index score; and/or (iii) Scleroderma skin patient-reported outcome score. In one embodiment, an increase in one or more measures relative to baseline is achieved in the human subject, wherein the one or more measures are selected from: (i) Transition dyspnoea index score; and/or (ii) Medical outcome study 36-item short form score.

In one embodiment, the human subject is characterised by one or more of: (i) Presence of thickened skin with a modified Rodnan Skin Score ≥10 points, optionally, ≥15 points; (ii) Anti-topoisomerase I (anti-Scl70) autoantibody positive; (iii) Anti-RNA polymerase autoantibody positive; (iv) Anti-centromere autoantibody negative; and (v) Disease duration ≤7 years. In one embodiment, the human subject is anti-topoisomerase I (anti-Scl70) autoantibody positive. In one embodiment, the human subject is characterised by: (i) Presence of thickened skin with a modified Rodnan Skin Score ≥10 points, optionally, ≥15 points; (ii) Disease duration ≤7 years; and (iii) Anti-topoisomerase I (anti-Scl70) autoantibody positive. In one embodiment, the human subject is characterised by: (i) Presence of thickened skin with a modified Rodnan Skin Score ≥10 points, optionally, ≥15 points; (ii) Disease duration ≤7 years; and (iii) Anti-RNA polymerase autoantibody positive. In one embodiment, the human subject is characterised by: (i) Presence of thickened skin with a modified Rodnan Skin Score 10 points, optionally, ≥15 points; (ii) Disease duration ≤7 years; and (iii) Anti-centromere polymerase autoantibody negative. In one embodiment, the human subject is characterised by: (i) Presence of thickened skin with a modified Rodnan Skin Score ≥10 points, optionally, ≥15 points; and (ii) Anti-topoisomerase I (anti-Scl70) autoantibody positive.

In one embodiment, the human subject has a disease duration of ≥2 years and one or more of: (i) Reduction in forced vital capacity; (ii) Reduction in diffusing capacity of the lung for carbon monoxide; (iii) Anti-topoisomerase I (anti-Scl70) autoantibody positive; and/or (iv) Increased extent of interstitial lung disease on high-resolution computed tomography.

In one embodiment, the anti-BlyS antibody comprises: CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6 or variants thereof. In one embodiment, the anti-BlyS antibody comprises: CRDH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6. In one embodiment, the anti-BlyS antibody comprises a variable heavy chain sequence of SEQ ID NO: 7, or a sequence which is at least 90% identical thereto, and a variable light chain sequence of SEQ ID NO: 8 or a sequence which is at least 90% identical thereto. In one embodiment, the anti-BlyS antibody comprises a variable heavy chain sequence of SEQ ID NO: 7 and a variable light chain sequence of SEQ ID NO: 8. In one embodiment, the anti-BlyS antibody is belimumab.

In one embodiment, the anti-BlyS antibody is administered subcutaneously. In one embodiment, the anti-BlyS antibody is administered to a human subject at a unit dose of 200 mg per week. In one embodiment, the anti-BlyS antibody is co-administered with an additional immunosuppressive agent. In one embodiment, the additional immunosuppressive agent is selected from methotrexate, mycophenolate mofetil, mycophenolate sodium, azathioprine, a corticosteroid, a calcineurin inhibitor, a pyrimidine synthesis inhibitor, and/or an anti-malarial agent.

In one aspect, there is provided a pharmaceutical composition comprising the anti-BlyS antibody according to the invention and a pharmaceutically acceptable excipient, for use in the treatment of connective tissue disease-associated interstitial lung disease. In one embodiment, the connective tissue disease-associated interstitial lung disease is systemic sclerosis-associated interstitial lung disease.

In one aspect, there is provided use of the anti-BlyS antibody according to the invention for the manufacture of a medicament for use in the treatment of connective tissue disease-associated interstitial lung disease. In one embodiment, the connective tissue disease-associated interstitial lung disease is systemic sclerosis-associated interstitial lung disease.

In one aspect, there is provided a method for the treatment of connective tissue disease-associated interstitial lung disease in a human subject in need thereof comprising administering to the human subject a therapeutically effective amount of the anti-BlyS antibody according to the invention. In one embodiment, the connective tissue disease-associated interstitial lung disease is systemic sclerosis-associated interstitial lung disease.

In one aspect, there is provided a method of improving the quality of life of a subject suffering from connective tissue disease-associated interstitial lung disease, wherein the human subject is administered a therapeutically suitable amount of the anti-BlyS antibody according to the invention. In one embodiment, the connective tissue disease-associated interstitial lung disease is systemic sclerosis-associated interstitial lung disease.

In one aspect, there is provided a BlyS antagonist for use in the treatment of autoimmune interstitial lung disease. In one embodiment, the autoimmune interstitial lung disease comprises connective tissue disease-associated interstitial lung disease. In one aspect, there is provided a BlyS antagonist for use in the treatment of Connective Tissue Disease, such as Connective tissue disease associated Interstitial Lung disease (CTD-ILD).

The term “connective tissue disease” or “CTD” as used herein refers to a group of autoimmune disorders, which comprise systemic sclerosis (SSc), rheumatoid arthritis (RA), Sjögren's syndrome (SS), an idiopathic inflammatory myopathy (IIM), mixed connective tissue disease (MCTD), and systemic lupus erythematosus (SLE). In one embodiment, the IIM comprises polymyositis, dermatomyositis, and/or anti-synthetase syndrome. A further term for “connective tissue disease” is “systemic autoimmune rheumatic disease”. In one embodiment, the CTD is diagnosed in accordance with a set of internationally recognised classification criteria. In one embodiment, the internationally recognised classification criteria are the American College of Rheumatology (ACR) and/or the European Alliance of Associations for Rheumatology (EULAR) classification criteria. In one embodiment, the CTD is diagnosed using a set of classification criteria as set out in Table 1 below:

TABLE 1
CTD Classification Criteria

| Disease | Classification Criteria |
|---|---|
| Systemic sclerosis | ACR/EULAR 2013 SSc Classificiation |
| | Criteria (van den Hoogen F, et al. 2013 |
| | Arthritis Rheum. 2013;65(11):2737-47). |
| Rheumatoid arthritis | ACR/EULAR 2010 RA Classification Criteria |
| | (Aletaha D, et al. Ann Rheum Dis |
| | 2010;69:1580-1588) |
| Sjogren's syndrome | EULAR/ACR 2016 pSS Classification Criteria |
| | (Shiboski CH, et al. 2016 Arthritis Rheumatol. |
| | 2017;69(1):35-45). |
| Idiopathic inflammatory myopathy | EULAR/ACR 2019 IIM Classification Criteria |
| | (Lundberg I E, et al. Ann Rheum Dis. 2017; |
| | 76(12): 1955-1964). |
| Mixed connective tissue disease | Japan research committee of the MHLW |
| | (ministry of health, labor, and welfare) for |
| | systemic autoimmune diseases 2019 MCTD |
| | Classification Criteria (Tanaka Y, et al. 2019 |
| | Modern Rheumatology, Volume 31, 2021, |
| | Pages 29-33). |
| Systemic lupus erythematosus | ACR/EULAR 2019 SLE Classification |
| | Criteria SLE (Aringer M, et al. Ann Rheum |
| | Dis.2019;78(9): 1151-59). |

In one aspect, there is provided a BlyS antagonist for use in the treatment of Systemic sclerosis (SSc).

Systemic sclerosis is divided into two broad categories: limited and diffuse cutaneous disease. Diffuse cutaneous disease (dcSSc), involves skin thickening proximal to the elbows and knees, both of which are associated with a broad range of systemic organ manifestations affecting the lungs, heart, gastrointestinal tract, and kidney. While the relative risk of developing these internal organ manifestations varies across the cutaneous subgroups the pathophysiology underlying each specific organ manifestation is the same and rate of disease progression is similar. For example, diffuse cutaneous subset and presence of ATA antibodies are associated with greater risk of developing ILD; however, ILD patterns on imaging, such as high-resolution computed tomography (HRCT), do not differ between the two cutaneous subsets or across autoantibody subgroups (Bellia, et al., (2009) La Radiologia Medica. 114(2):190-203; Patiwetwitoon, et al., (2012) Journal of Clinical Rheumatology. 18(5):p 229-233). Furthermore, in the majority of cases ILD develops within the first 3-5 years from disease onset, regardless of cutaneous subset (Nihtyanova & Denton, (2020) Journal of Scleroderma and Related Disorders. 5(2S):p 6-16). Moreover, although extent of skin involvement is different between the two cutaneous subsets, pathology in involved skin are similar in the two cutaneous subsets (Bosello, et al., (2018) Journal of Clinical Pathology. 71:620-625.).

Therapies that work for those with the diffuse cutaneous subtype should therefore also impact those subjects suffering from the limited cutaneous form of the disease. As such, in one aspect of the invention, there is provided a BlyS antagonist for use in the treatment of a human subject with limited cutaneous systemic sclerosis. In a further aspect, the subject to be treated is characterised by the presence of thickened skin with a modified Rodnan skin score (mRSS) >0 points over at least one area distal to the knees and/or elbows. In a further aspect, the subject to be treated is characterised by the presence of thickened skin with an mRSS >0, >1, >2, >3, >4, >5, >6, >7, >8, >9, or >10 points.

In one aspect of the invention, there is provided a BlyS antagonist for use in the treatment of diffuse cutaneous systemic sclerosis. In a further aspect, the subject to be treated is characterised by the presence of thickened skin with a modified Rodnan skin score >0 points over at least one skin area proximal to the knees and/or elbows, for example, in addition to distal areas. In a further aspect, the subject to be treated is characterised by the presence of thickened skin with a modified Rodnan skin score >0, >1, >2, >3, >4, >5, >6, >7, >8, >9, >10, >11, >12, >13, >14, >15, >20, >25, or >30 points proximal to the knees and/or elbows in addition to the distal areas. In one aspect, the subject to be treated is characterised by the presence of thickened skin with a modified Rodnan skin score of 15-51 points.

In one aspect, the subject to be treated is characterised by thickened skin with an mRSS >0 points both over at least one area distal to the knees and/or elbows, and over at least one area proximal to the knees and/or elbows. In a further aspect, the subject to be treated is characterised by the presence of thickened skin with an mRSS >0, >1, >2, >3, >4, >5, >6, >7, >8, >9, >10, >11, or >12 points distal to the knees and/or elbows, and an mRSS >0, >1, >2, >3, >4, >5, >6, >7, >8, >9, >10, >11, or >12 points proximal to the knees/elbows.

The Modified Rodnan Skin Score (mRSS) is a well characterised method for measuring the severity of skin manifestations in Systemic sclerosis. Total mRSS is calculated as the sum of the scores of 17 body areas, including fingers, back of hands, forearms, upper arms, face, anterior chest, abdomen, thighs, legs, and feet (Khanna, et al., (2017) J Scleroderma Relat Disord. 2017; 2(1):11-8.). Each area is scored between 0 and 3 points, where mRSS score indicates:

| | |
|---|---|
| 0 | Normal skin |
| 1 | Definite but mild skin thickness, where the |
| | examiner can easily make skin folds between |
| | 2 fingers |
| 2 | Moderate skin thickness with difficulty in |
| | making skin folds |
| 3 | Severe skin thickness with inability to make |
| | skin folds between 2 fingers |

These individual values are added, and the sum is defined as the total skin score with a maximum score of 51 points.

Clinically meaningful disease progression in SSc has been defined by an increase in modified Rodnan skin score by at least 20%, or at least 5 points relative to baseline. In one aspect, the disease progression of SSc in a subject is characterised by an increase in modified Rodnan skin score of ≥20% relative to baseline. In one aspect, the disease progression of SSc in a subject is characterised by an increase in modified Rodnan skin score of ≥5 points relative to baseline.

In one aspect, the subject to be treated is characterised by diagnosis with interstitial lung disease (ILD) associated with SSc. In one aspect, the subject to be treated is characterised by diagnosis with interstitial lung disease (ILD) associated with a connective tissue disease.

The term “interstitial lung disease” or “ILD” as used herein refers to a disease characterised by non-infective inflammation and/or fibrosis affecting the lung parenchyma.

ILD is a disease manifestation which occurs in association with most connective tissue diseases including rheumatoid arthritis (RA-ILD), idiopathic inflammatory myopathies (IIM-ILD), such as polymyositis (PM-ILD), dermatomyositis (DM-ILD), and anti-synthetase syndrome-ILD, Systemic Lupus Erythematous (SLE-ILD), Sjögren's Syndrome (SS-ILD), Systemic sclerosis (SSc-ILD), and mixed connective tissue disease (MCTD-ILD). Despite differences in the pathophysiology of the underlying CTD, the ILD associated with each disease share similar pathogenesis driven by underlying immune system dysfunction and immune-mediated pulmonary inflammation. In one aspect, there is therefore provided a BLyS antagonist for the treatment of ILD, for example, CTD-ILD. In one aspect, there is provided a BlyS antagonist for use in the treatment of an inflammatory-driven ILD, for example, an inflammatory-driven CTD-ILD.

Interstitial lung disease is a common manifestation of SSc, and of other CTDs, that results from vascular injury resulting in aberrant inflammation and fibrosis of lung tissue. SSc-ILD is among the leading causes of SSc related death, and approximately one third of lcSSc subjects develop ILD, while more than half of subjects with dcSSc can develop ILD. In a subject with ILD, lung function is impacted through inflammation and damage through autoimmunity, vascular dysfunction leading to reduced blood flow to lung tissue, and fibrosis through excessive activation of fibroblasts.

As such, in a further aspect, there is provided a BlyS antagonist for use in the treatment of connective tissue disease-associated interstitial lung disease (CTD-ILD).

In one aspect, there is provided a BlyS antagonist for use in the treatment of CTD-ILD, wherein the CTD-ILD comprises systemic sclerosis-associated interstitial lung disease (SSc-ILD), rheumatoid arthritis-associated interstitial lung disease (RA-ILD), Sjögren's syndrome-associated interstitial lung disease (SS-ILD), an idiopathic inflammatory myopathy-associated interstitial lung disease (IIM-ILD), and/or mixed connective tissue disease-associated interstitial lung disease (MCTD-ILD).

In one aspect, there is provided a BlyS antagonist for use in the treatment of CTD-ILD, wherein the CTD-ILD comprises SSc-ILD, RA-ILD, SS-ILD, IIM-ILD, and/or MCTD-ILD, and wherein the CTD-ILD does not comprise systemic lupus erythematosus-associated interstitial lung disease (SLE-ILD).

In one embodiment, the aforementioned SSc-ILD, RA-ILD, SS-ILD, IIM-ILD, and/or MCTD-ILD comprises an overlap syndrome. That is to say, the CTD-ILD comprises a CTD disorder as a primary diagnosis and a further CTD disorder as a secondary diagnosis. In one embodiment, the primary diagnosis is the CTD that was diagnosed first and/or is the most predominant. In one embodiment, the SSc-ILD comprises SSc as the primary diagnosis and a further CTD disorder as the secondary diagnosis; the RA-ILD comprises RA as the primary diagnosis and a further CTD disorder as the secondary diagnosis; the IIM-ILD comprises an IIM as the primary diagnosis and a further CTD disorder as the secondary diagnosis; and/or the MCTD comprises MCTD as the primary diagnosis and a further CTD as the secondary diagnosis.

In one embodiment, the CTD-ILD is not systemic lupus erythematosus-associated interstitial lung disease (SLE-ILD). Thus, in one embodiment, the CTD-ILD does not comprise SLE as the primary diagnosis. In one embodiment, the CTD does not comprise SLE as the primary and/or the secondary diagnosis. Thus, in one embodiment, the CTD does not comprise SLE. In one embodiment, the SSc-ILD does not further comprise SLE.

In one aspect, there is provided a BlyS antagonist for use in the treatment of CTD-ILD, wherein the CTD-ILD comprises SSc-ILD, RA-ILD, SS-ILD, IIM-ILD, and/or MCTD-ILD, and wherein the SSc-ILD does not further comprise SLE or SLE-ILD.

In one embodiment, there is provided a BlyS antagonist for use in the treatment of CTD-ILD, wherein the CTD-ILD consists of SSc-ILD, RA-ILD, SS-ILD, IIM-ILD, and/or MCTD-ILD.

In one aspect, there is provided an anti-BlyS antagonist for use in the treatment of systemic sclerosis-associated ILD (SSc-ILD). In a further aspect, the ILD is associated with limited cutaneous systemic sclerosis (lcSSc-ILD). In a further aspect, the ILD is associated with diffuse cutaneous systemic sclerosis (dcSSc-ILD).

Several methods to diagnose ILD exist including, but not limited to, imaging tests, pulmonary function tests (PFTs), and lung tissue biopsy. Imaging tests include chest X-rays, high-resolution computed tomography (HRCT) scans, and magnetic resonance imaging (MRI) and can detect changes in lung tissues such as fibrosis, nodules, honeycombing, and ground glass opacities. HRCT scans are the reference standard for early diagnosis of CTD-ILD, for example, SSc-ILD, with quantitative HRCT allowing quantification of CTD-ILD (e.g., SSc-ILD), lung involvement and of fibrosis (Khanna, et al., (2021) Arthritis Rheumatol. 2022; 74(1):13-27).

In one aspect, the present disclosure provides a BlyS antagonist for use in the treatment of a subject diagnosed with CTD-ILD, for example, SSc-ILD, wherein diagnosis is confirmed through imaging tests selected from the group of X-rays, HRCT scans, and MRI scans. In a further aspect, diagnosis of CTD-ILD, for example, SSc-ILD, in the subject is confirmed by HRCT scans.

In one aspect, the present disclosure provides a BlyS antagonist for use in the treatment of a subject with CTD-ILD, for example, SSc-ILD, wherein diagnosis is confirmed through one or more pulmonary function tests selected from the group including Forced Vital Capacity (FVC), Total Lung Capacity (TLC), and/or Diffusing Capacity of the Lung for Carbon Monoxide (DLco). In a further aspect, diagnosis with SSc-ILD is confirmed with FVC, FVC, and TLC; FVC and DLco; or TLC and DLco. In one aspect, diagnosis with CTD-ILD is confirmed with FVC and/or DLco.

The terms “diffusing capacity of the lung for carbon monoxide”, “diffusing capacity for carbon monoxide”, and “DLco” are used herein interchangeably.

Pulmonary function testing is a procedure used for monitoring of lung involvement in CTD subjects, such as SSc subjects (Hoffmann-Vold, et al., (2021) ERJ Open Res. 2021; 7(1): 00235-2020) and Forced Vital Capacity (FVC) is globally accepted for assessment of treatment effects in subjects with ILD. FVC was also unanimously endorsed by the OMERACT Connective Tissue Disease-Interstitial Lung Disease Working Group on Lung Physiology for use in randomized clinical trials (Roofeh, et al., (2021) Seminars in Arthritis and Rheumatism. 2021; 51(6): 1331-1341). In ILD, FVC reflects loss of functional lung tissue and lung volume, with low FVC and decline in FVC over time being associated with increased mortality.

In one embodiment, the ILD comprises one or more of the following ILD patterns: nonspecific interstitial pneumonia (NSIP), usual interstitial pneumonia (UIP), organising pneumonia (OP), lymphocytic interstitial pneumonia (LIP), and/or diffuse alveolar damage (DAD). In one embodiment, the ILD comprises two or more of the aforementioned ILD patterns. In one embodiment, the ILD comprises NSIP and UIP. In one embodiment, the ILD comprises NSIP and OP. In one embodiment, the ILD comprises an indeterminant pattern. In one embodiment, the ILD pattern is diagnosed in accordance with a set of internationally recognised classification criteria. In one embodiment, the set of internationally recognised classification criteria comprise the American Thoracic Society/European Respiratory Society (ATS/ERS) criteria. In one embodiment, the ILD pattern is diagnosed in accordance with the American Thoracic Society/European Respiratory Society (ATS/ERS) 2002 consensus classification. In one embodiment, the ILD pattern is diagnosed by radiology. In one embodiment, the ILD pattern is diagnosed by high-resolution computed tomography (HRCT).

In one embodiment, the ILD pattern is UIP and diagnosis is by HRCT; imaging features of UIP comprise honeycombing, reticulation, and/or traction bronchiectasis (Raghu G, et al. Am J Respir Crit Care Med. 2022; 205(9):e18-e47; Travis W D, et al. Am J Respir Crit Care Med 2008; 177: 1338-1347). In one embodiment, the ILD pattern is definitive UIP or probable UIP. In an alternative embodiment, the UIP pattern is indeterminant for UIP. In one embodiment, the UIP pattern is diagnosed by HRCT in accordance with Table 2 below. In one embodiment, the ILD pattern is NSIP and diagnosis is by HRCT; imaging features of NSIP comprise basal predominant reticular abnormalities with traction bronchiectasis, peri-bronchovascular extension, and subpleural sparing, frequently associated with ground-glass attenuation (Fischer A, et al. Eur Respir J. 2015 October; 46(4):976-87; Travis W D, et al. Am J Respir Crit Care Med 2008; 177: 1338-1347). In one embodiment, the ILD pattern is OP and diagnosis is by HRCT; imaging features of OP comprise basal predominant consolidation, often peri-diaphragmatic, associated with features of fibrosis (for example, traction bronchiectasis, reticular abnormality, or lower lobe volume loss) (Fischer A, et al. Eur Respir J. 2015 October; 46(4):976-87). In one embodiment, the ILD pattern is LIP and diagnosis is by HRCT; imaging features of LIP comprise predominantly peri-bronchovascular cysts, with or without ground glass opacities or reticular abnormalities (Fischer A, et al. Eur Respir J. 2015 October; 46(4):976-87).

In one embodiment, the ILD pattern is associated with inflammation and/or fibrosis of the lung. In one embodiment, the ILD pattern is OP, LIP, and/or NSIP and is associated with inflammation of the lung. In one embodiment, the ILD pattern is NSIP and/or UIP and is associated with inflammation and/or fibrosis of the lung. In one embodiment, the ILD pattern is UIP and is associated with fibrosis of the lung.

It has been reported that the prevalence of the aforementioned ILD patterns varies depending on the underlying CTD (Joy G M, et al. European Respiratory Review 2023; 32(167):220210). Thus, in one embodiment, an ILD pattern is associated with a particular CTD in a patient with CTD-ILD. In one embodiment, the CTD comprises rheumatoid arthritis and the ILD pattern comprises UIP. In one embodiment, the CTD comprises systemic sclerosis, Sjögren's syndrome, an idiopathic inflammatory myopathy, systemic lupus erythematosus, and/or mixed connective tissue disease and the ILD pattern comprises NSIP. In one embodiment, the CTD comprises systemic sclerosis, Sjögren's syndrome, an idiopathic inflammatory myopathy, and/or mixed connective tissue disease and the ILD pattern comprises NSIP. In one embodiment, the CTD comprises Sjögren's syndrome and the ILD pattern comprises LIP. In one embodiment, the CTD comprises an idiopathic inflammatory myopathy and the ILD pattern comprises OP. In one embodiment, the idiopathic inflammatory myopathy comprises polymyositis, dermatomyositis, or anti-synthetase syndrome.

In one embodiment, the patient with CTD-ILD exhibits an ILD pattern that comprises UIP. In one embodiment, the UIP is a definitive UIP and/or probable UIP. In one embodiment, the definitive UIP and/or probable UIP is diagnosed by radiology, optionally, HRCT. In a further embodiment, the patient with CTD-ILD exhibits an ILD pattern that does not comprise UIP. In one embodiment, the ILD pattern does not comprise a definitive UIP and/or probable UIP. In one embodiment, the ILD pattern is indeterminate for UIP. In one embodiment, the absence of definitive UIP and/or probable UIP, or the indeterminate for UIP pattern, is diagnosed by radiology, optionally, HRCT. In one embodiment, the UIP pattern is diagnosed by HRCT in accordance with Table 2 below.

TABLE 2
HRCT pattern of UIP

| | | | Indeterminate |
|---|---|---|---|
| | UIP Pattern (Definitive) | Probable UIP Pattern | for UIP |
| Level of | Confident (>90%) | Provisional high confidence | Provisional low |
| confidence | | (70-89%) | confidence |
| for UIP | | | (51-69%) |
| histology | | | |
| Distribution | Subpleural and basal | Subpleural and basal | Diffuse |
| | predominant | predominant | distribution |
| | Often heterogeneous (areas of | Often heterogeneous (areas of | without |
| | normal lung interspersed with | normal lung interspersed with | subpleural |
| | fibrosis) | reticulation and traction | predominance |
| | Occasionally diffuse | bronchiectasis/bronchiolectasis) | |
| | May be asymmetric | | |
| CT features | Honeycombing with or | Reticular pattern with traction | CT features of |
| | without traction | bronchiectasis/bronchiolectasis | lung fibrosis |
| | bronchiectasis/bronchiolectasis | May have mild ground-glass | that do not |
| | Presence of irregular | opacity | suggest any |
| | thickening of interlobular septa | Absence of subpleural sparing | specific etiology |
| | Usually superimposed with a | | |
| | reticular pattern, mild ground- | | |
| | glass opacity | | |
| | May have pulmonary | | |
| | ossification | | |

(Adapted from Raghu G, et al. Am J Respir Crit Care Med. 2022; 205(9): e18-e47)

In one aspect, the present disclosure provides a BlyS antagonist for use in the treatment of a subject diagnosed with SSc-ILD, wherein the subject is characterised by thickened skin with a modified Rodnan skin score ≥10 points, optionally, ≥15 points.

In one aspect, the present disclosure provides a BlyS antagonist for use in the treatment of a subject diagnosed with SSc-ILD by HRCT scans, wherein the subject is characterised by thickened skin with a modified Rodnan skin score ≥10 points, optionally, ≥15 points.

Autoantibody profiles are used as one of the factors to assess prognosis of patients with SSc and SSc ILD. For example, patients who are anti-topoisomerase (ATA) (anti-Scl70) and/or anti-RNA polymerase (ARA) autoantibodies are most likely to develop ILD. Conversely, patients positive with anti-centromere (ACA) autoantibodies are at low risk of developing ILD and tend to belong to the lcSSc cutaneous subtype. Methods of determining presence or absence of autoantibodies in a subject are well known to a person skilled in the art.

In one aspect, the present disclosure provides a BlyS antagonist for use in the treatment of a subject diagnosed with SSc-ILD, wherein the subject is anti-topoisomerase (anti-Scl70) autoantibody positive. In one aspect, the present disclosure provides a BlyS antagonist for use in the treatment of a subject diagnosed with dcSSc-ILD, wherein the subject is anti-topoisomerase (anti-Scl70) autoantibody positive.

In one aspect, there is provided a BlyS antagonist for use in the treatment of a subject with SSc-ILD, for example, dcSSc-ILD, wherein the subject is anti-RNA polymerase autoantibody positive.

In an alternative aspect, there is provided a BlyS antagonist for use in the treatment of a subject with SSc-ILD, for example, dcSSc-ILD, wherein the subject is anti-centromere autoantibody negative.

In one aspect, the present disclosure provides a BlyS antagonist for use in the treatment of a subject diagnosed with SSc-ILD by HRCT scans, wherein the subject is anti-topoisomerase (anti-Scl70) autoantibody positive.

In one aspect, the present disclosure provides a BlyS antagonist for use in the treatment of a subject diagnosed with SSc-ILD, wherein:
(i) The subject is anti-topoisomerase (anti-Scl70) autoantibody positive; and
(ii) The subject is characterised by thickened skin with a modified Rodnan skin score ≥10 points, optionally, ≥15 points.

In one aspect, the present disclosure provides a BlyS antagonist for use in the treatment of a subject diagnosed with SSc-ILD by HRCT scans, wherein:
(i) The subject is anti-topoisomerase (anti-Scl70) autoantibody positive; and
(ii) The subject is characterised by thickened skin with a modified Rodnan skin score ≥10 points, optionally, ≥15 points.

In another embodiment, the aforementioned subject is characterised by thickened skin with a modified Rodnan skin score of ≥1, ≥2, ≥3, ≥4, ≥5, ≥6, ≥7, ≥8, ≥9, ≥10, ≥11, ≥12, ≥13, ≥14, or ≥15 points.

Interstitial lung disease is a disease manifestation associated with connective tissue diseases, such as systemic sclerosis, rheumatoid arthritis, Sjögren's syndrome, systemic lupus erythematosus, idiopathic inflammatory myopathies (e.g., polymyositis, dermatomyositis, anti-synthetase syndrome), and mixed connective tissue disease. ILD is characterised by inflammation or scarring (fibrosis) of the lungs, or both, which can prevent oxygen from being absorbed in the lung, leading to shortness of breath, cough, and fatigue. The natural history of ILD is variable regardless of background CTD, some patients experience accelerated loss of lung function, and others progress slowly or exhibit stable disease. There are no validated predictors of progression and therefore patients need to be closely monitored and treated accordingly. Outcome Measures in Rheumatology (OMERACT) has defined clinically meaningful progression for CTD-ILD by a relative decline in FVC of ≥10% or a 5% to <10% relative decline in FVC % and 15% relative decline in DLco %. This definition has also been applied to SSc-ILD. Recently, the American Thoracic Society has defined clinically meaningful progression for ILD as the presence of at least two of the following three criteria occurring within one year: worsening respiratory symptoms; physiological evidence of disease progression, including one or both of: absolute decline in forced vital capacity of at least 5% predicted or absolute decline in diffusing capacity for carbon monoxide of at least 10% predicted; or radiological evidence of disease progression.

As such, in one aspect, there is provided a BlyS antagonist for use in the treatment of progressive CTD-ILD, for example, progressive SSc-ILD. In a further aspect, the invention provides a BlyS antagonist for use in the treatment of progressive lcSSc-ILD. In another aspect, the invention provides an anti-BlyS antagonist for use in the treatment of progressive dcSSc-ILD. In a further aspect, the invention provides an anti-BlyS antibody for use in the treatment of progressive dcSSc-ILD.

In one aspect, progression of lung function decline (or interstitial lung disease progression) in a human subject diagnosed with SSc-ILD, for example, with dcSSc-ILD or lcSSc-ILD, is characterised by a reduction in forced vital capacity of at least 10% (≥10%) relative to baseline. In one aspect, progression of lung function decline (or interstitial lung disease progression) in a human subject diagnosed with CTD-ILD is characterised by a reduction in forced vital capacity of at least 10% (≥10%) relative to baseline. In a further embodiment, progression of lung function decline (or interstitial lung disease progression) is characterised by a reduction in forced vital capacity of at least 5%, at least 6%, at least 7%, at least 8%, or at least 9% (≥5%, ≥6%, ≥7%, ≥8%, or ≥9%) relative to baseline.

Throughout the specification the term “baseline” is used to define a previous measurement against which it is possible to identify or record a change in the same clinical endpoint or physician reported outcome at a later timepoint. In one embodiment, the previous measurement against which the comparison is made is taken 3, 6, 12, 24, or 36 months earlier. In one embodiment, the previous measurement against which the comparison is made is taken 8, 10, 12, 20, 30, 40, 50, 51, 52, 53, 54, 55, 56, 57, 58, 59, 60, 70, 80, 90, 100, 101, 102, 103, 104, 105, or 110 weeks earlier. In one embodiment, the previous measurement against which the comparison is made is taken 52 weeks or 104 weeks earlier. In one embodiment, the “baseline” measurement is taken prior to or within 1, 2, 5, or 7 days of a first administration of a BlyS antagonist.

In another aspect, disease progression, for example, interstitial lung disease progression, is characterised by a reduction in forced vital capacity of 0.1-<10%, 0.1-9%, 0.1-8%, 0.1-7%, 0.1-6%, 0.1-5%, 0.1-4%, 0.1-3%, 0.1-2%, or 0.1-1% relative to baseline.

In one aspect, progression of lung function decline is measured in absolute decline in FVC ≥5% as determined by a comparison of the screening lung function test and a previous lung function test done within 12 months prior to screening and worsening respiratory symptoms, or absolute decline in DLco (corrected for Hb) ≥10% predicted as determined by a comparison of the screening lung function test and a previous lung function test done within 12 months prior to screening and worsening respiratory symptoms.

In another aspect, progression of lung function decline (or interstitial lung disease progression) is characterised by a relative reduction in FVC ≥5% to <10% and a relative reduction in carbon monoxide diffusing capacity (DLco) of at least 15% relative to baseline. Thus, in one embodiment, progression of lung function decline (or interstitial lung disease progression) in a human subject diagnosed with CTD-ILD, for example, SSc-ILD, is characterised by a reduction in forced vital capacity (FVC) of at least 5% to less than 10% (≥5% to <10%) relative to baseline and a reduction in diffusing capacity of the lung for carbon monoxide (DLco) of at least 15% (≥15%) relative to baseline. In a further aspect, disease progression is characterised by a reduction in carbon monoxide diffusing capacity (DLco) of at least 15% relative to baseline. For example, in one aspect, the reduction in lung function decline is characterised by a reduction in carbon monoxide diffusing capacity (DLco) of <15%, for example, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1% relative to baseline.

In a further aspect, the reduction in lung function decline is characterised by a reduction in carbon monoxide diffusing capacity (DLco) of 0.1-<15%, 0.1-14%, 0.1-13%, 0.1-12%, 0.1-11%, 0.1-10%, 0.1-9%, 0.1-8%, 0.1-7%, 0.1-6%, 0.1-5%, 0.1-4%, 0.1-3%, 0.1-2%, or 0.1-1% relative to baseline.

In a further aspect, the invention provides a BlyS antagonist for use in the treatment of progressive SSc-ILD or progressive CTD-ILD, wherein progression of lung disease (or interstitial lung disease progression) is characterised by either:
(i) a reduction in forced vital capacity of at least 5% (≥5%) or at least 10% (≥10%) relative to baseline; or
(ii) a reduction in forced vital capacity of at least 5% relative to baseline and a reduction in carbon monoxide diffusing capacity of at least 15% relative to baseline.

In a further aspect, there is provided a BlyS antagonist for use in the treatment of progressive SSc-ILD, wherein the disease progression is further characterised by an increase in modified Rodnan skin score ≥20% relative to baseline and one of:
(i) A reduction in forced vital capacity ≥10% relative to baseline; or
(ii) A reduction in forced vital capacity ≥5% relative to baseline and a reduction in carbon monoxide diffusing capacity of ≥15% relative to baseline.

In another aspect, there is provided a BlyS antagonist for use in the treatment of progressive CTD-ILD, wherein the progressive CTD-ILD comprises interstitial lung disease progression. In one embodiment, the interstitial lung disease progression is characterised by two or more of the following (a) to (c) occurring within a 12 month time period:
(a) a worsening of respiratory symptoms;
(b) a physiological evidence of interstitial lung disease progression, wherein the physiological evidence of interstitial lung disease progression comprises:
   (i) an absolute reduction in forced vital capacity of ≥5% predicted; and/or
   (ii) an absolute reduction in diffusing capacity of the lung for carbon monoxide of ≥10% predicted; and/or
(c) a radiological evidence of interstitial lung disease progression.

In one embodiment, the worsening of respiratory symptoms comprises a worsening breathlessness and/or cough. In one embodiment, the physiological evidence of interstitial lung disease progression comprises an absolute reduction in forced vital capacity of ≥1%, ≥2%, ≥3%, ≥4%, ≥5%, ≥6%, ≥7%, ≥8%, ≥9%, or ≥10% predicted; and/or an absolute reduction in diffusing capacity of the lung for carbon monoxide of ≥5%, ≥6%, ≥7%, ≥8%, ≥9%, ≥10%, ≥11%, ≥12%, ≥13%, ≥14%, ≥15% predicted. In one embodiment, the radiological evidence of interstitial lung disease progression is based on an HRCT scan. In one embodiment, the radiological evidence of interstitial lung disease progression comprises a combined assessment of reticulation and/or fibrosis and/or ground glass opacity.

In one embodiment, disease progression comprises connective tissue disease progression and/or interstitial lung disease progression. In one embodiment, the connective tissue disease progression comprises SSc disease progression. In one embodiment, the SSc progression can also be characterised by the development of further SSc attributed complications. In a further aspect, there is provided a BlyS antagonist for use in the treatment of progressive SSc-ILD, wherein the disease progression (for example, the SSc disease progression) is further characterised by:
(i) A reduction in forced vital capacity of at least 10% relative to baseline; or
(ii) A reduction in forced vital capacity of at least 5% relative to baseline and a reduction in carbon monoxide diffusing capacity of at least 15% relative to baseline; or
(iii) A ≥20% increase in mRSS relative to baseline; or
(iv) New scleroderma renal crisis; or
(v) New onset of pulmonary arterial hypertension on right heart catheterization, requiring treatment; or
(vi) New onset of left ventricular failure, defined as left ventricular ejection fraction ≤45%, requiring treatment; or
(vii) Gastrointestinal dysmotility, requiring enteral or parenteral nutrition; or
(viii) Ischemic digital ulcer, requiring hospitalization, or development of critical digital ischemia, gangrene, or amputation; or
(ix) Any cause of death (regardless of association with SSc).

In a further aspect, there is provided a BlyS antagonist for use in the treatment of progressive SSc-ILD in a subject, wherein the subject is anti-topoisomerase (anti-Scl70) autoantibody positive, and wherein the disease progression is further characterised by either:
(i) A reduction in forced vital capacity ≥10% relative to baseline; or
(ii) A reduction in forced vital capacity ≥5% relative to baseline and a reduction in carbon monoxide diffusing capacity of ≥15% relative to baseline.

In one aspect, the invention provides a BlyS antagonist for use in the treatment of progressive CTD-ILD, wherein the progressive CTD-ILD comprises interstitial lung disease progression, and wherein the interstitial lung disease progression is characterised by any one or more of:
(i) A reduction in forced vital capacity of ≥10% relative to baseline;
(ii) A reduction in forced vital capacity of ≥5% to <10% relative to baseline combined with worsening of respiratory symptoms;
(iii) A reduction in forced vital capacity of ≥5% to <10% relative to baseline combined with radiological evidence of interstitial lung disease progression; and/or
(iv) A reduction in diffusing capacity of the lung for carbon monoxide of ≥10% relative to baseline combined with radiological evidence of interstitial lung disease.

In a further embodiment, the reduction in forced vital capacity is ≥5%, ≥10%, ≥15%, ≥20%, ≥25%, or ≥30% relative to baseline. In one embodiment, the reduction in diffusing capacity of the lung for carbon monoxide is ≥5%, ≥10%, ≥15%, ≥20%, ≥25%, or ≥30% relative to baseline. In one embodiment, the worsening of respiratory symptoms comprises worsening breathlessness and/or cough. In one embodiment, the radiological evidence of interstitial lung disease progression is based on an HRCT scan. In one embodiment, the radiological evidence of interstitial lung disease progression comprises a combined assessment of reticulation and/or fibrosis and/or ground glass opacity. In one embodiment, the subject with progressive CTD-ILD is characterised by interstitial lung disease progression that has occurred within the previous 12, 24, or 36 months. In one embodiment, the interstitial lung disease progression has occurred within the previous 24 months. That is to say, in one embodiment, the baseline measurement is taken 24 months previously.

In one embodiment, the interstitial lung disease progression comprises progressive pulmonary fibrosis (PPF) (also termed “progressive fibrosing ILD”). In one embodiment, the PPF is characterised by any two or more of worsening respiratory symptoms, physiological evidence of disease progression, and/or radiological evidence of disease progression within 12 months. In one embodiment, the worsening respiratory symptoms comprise worsening breathlessness and/or cough. In one embodiment, the physiological evidence of disease progression comprises absolute decline in FVC ≥5% predicted within 12 months of follow-up and/or absolute decline in DLco (corrected for Hb) ≥10% predicted within 12 months of follow-up. In one embodiment, the radiological evidence of disease progression comprises radiological evidence of pulmonary fibrosis; for example, any one or more of: increased extent or severity of traction bronchiectasis and bronchiolectasis, new ground-glass opacity with traction bronchiectasis, new fine reticulation, increased extent or increased coarseness of reticular abnormality, new or increased honeycombing, and/or increased lobar volume loss (Raghu, G et al. Am J Respir Crit Care Med. 2022 May 1; 205(9):e18-e47). In one embodiment, the radiological evidence of disease progression is assessed by HRCT.

Disease progression can also be characterised by the development of further CTD attributed complications. In a further aspect, there is provided a BlyS antagonist for use in the treatment of progressive CTD-ILD, wherein the disease progression comprises a connective tissue disease progression. In one embodiment, the connective tissue disease progression comprises a worsening of existing disease manifestation(s) and/or a development of new extra-pulmonary disease manifestation(s). In one embodiment, the worsening of existing disease manifestation(s) and/or a development of new extra-pulmonary disease manifestation(s) requires treatment escalation. In one embodiment, the worsening of the existing disease manifestation(s) and/or the development of the new extra-pulmonary disease manifestation(s) comprises any one or more of:
(i) Development of a new extra-pulmonary CTD-related organ complication;
(ii) Deterioration of an existing extra-pulmonary CTD-related organ complication; and/or
(iii) Increase in overall disease activity as measured by an internationally recognised disease activity score (excluding lung domains), laboratory test, and/or clinical examination.

In one embodiment, (i) to (iii) above require an additional treatment intervention and/or treatment escalation. In one embodiment, the increase in overall disease activity is measured using the American College of Rheumatology (ACR) and/or European Alliance of Associations for Rheumatology (EULAR) criteria. In one embodiment, the extra-pulmonary CTD-related organ complication is an organ complication affecting any one or more of: joints, bones, muscle, heart, kidneys, gastrointestinal system, central nervous system, peripheral nervous system, vascular system, skin, liver, haematology, lymphatic system, salivary glands, and/or eye.

In one embodiment, the time to CTD progression as defined above is measured as progression-free survival.

In one aspect, there is provided a BlyS antagonist for use in treating a human subject with CTD-ILD, wherein the human subject is characterised by having an extent of ILD that is ≥10% of the whole lung. In one embodiment, the extent of ILD is assessed by HRCT. In one embodiment, the human subject is characterised by having ≥10% extent of ILD of the whole lung prior to a first administration of the BlyS antagonist. In one embodiment, the ≥10% extent of ILD of the whole lung comprises inflammation and/or fibrosis. In one embodiment, the ≥10% extent of ILD of the whole lung includes the combined assessment of reticulation/fibrosis and ground glass opacity. In a further embodiment, the extent of ILD is ≥1%, ≥2%, ≥3%, ≥4%, ≥5%, ≥6%, ≥7%, ≥8%, ≥9%, ≥10%, ≥11%, ≥12%, ≥13%, ≥14%, ≥15%, ≥20%, ≥25%, or ≥30% of the whole lung. In another embodiment, there is provided a BlyS antagonist for use in treating a human subject with CTD-ILD, wherein the human subject is characterised by having an extent of fibrosis that is ≥10% of the whole lung. In one embodiment, the extent of fibrosis is assessed by HRCT. In one embodiment, the human subject is characterised by having ≥10% extent of fibrosis of the whole lung prior to a first administration of the BlyS antagonist. In a further embodiment, the extent of fibrosis is ≥1%, ≥2%, ≥3%, ≥4%, ≥5%, ≥6%, ≥7%, ≥8%, ≥9%, ≥10%, ≥11%, ≥12%, ≥13%, ≥14%, ≥15%, ≥20%, ≥25%, or ≥30% of the whole lung.

The natural progression of SSc and SSc-ILD is highly variable (Man, et al., (2015) Rheumatology (Oxford). 2015; 54(8):1464-71); however, deterioration in lung function tends to occur early, in the first few years from diagnosis of SSc with 45% to 55% of SSc subjects showing deterioration in pulmonary function tests.

As such, in one aspect, there is provided a BlyS antagonist for use in treating a subject with early SSc, for example, with early SSc-ILD. In a further aspect, early SSc-ILD is characterised as a disease duration ≤7 years. In a further aspect, early SSc-ILD is characterised as a disease duration ≤5 years. In a further aspect, early SSc-ILD is characterised as a disease duration ≤3 years.

SSc-ILD patients most at risk of progressive lung disease display a combination of risk factors including early disease, high mRSS scores, extent of ILD at diagnosis, and autoantibody profile. Subject populations displaying multiple of these risk factors are therefore most likely to require timely intervention and respond to treatment.

Measurement of skin thickness is used as a surrogate for disease activity, severity, and mortality in patients with dcSSc. An increase in skin thickening is generally associated with new or worsening internal organ involvement, while severe skin disease that persists over time is associated with increased mortality (Steen & Medsger, (2001) Arthritis Rheum. 2001; 44(12):2828-35; Shand, et al., (2007) Arthritis Rheumatol. 2007; 56(7):2422-2431). Skin is therefore considered a very visible and accessible ‘window’ into the overall SSc disease process (Herrick, et al., (2022) Nat Rev Rheumatol 2022; 18: 276-285). As such, in one aspect, there is provided an anti-BlyS antagonist for use in the treatment of a human subject diagnosed with SSc, wherein the human subject is characterised by the presence of thickened skin with a modified Rodnan skin score ≥10 points, optionally, ≥15 points or is ATA positive.

In one aspect, there is provided a BlyS antagonist for use in the treatment of SSc, for example, with SSc-ILD, for example, progressive SSc-ILD, in a human subject, wherein the human subject is characterised by:
(i) Presence of thickened skin with a modified Rodnan Skin Score ≥10 points, optionally, ≥15 points;
(ii) Disease duration ≤7 years; and
(iii) Anti-topoisomerase I (anti-Scl70) autoantibody positive.

In one aspect, there is provided a BlyS antagonist for use in the treatment of a subject with SSc, for example, with SSc-ILD, for example, with progressive SSc-ILD, wherein the human subject is characterised by one or more of:
(i) Presence of thickened skin with a modified Rodnan skin score 10 points, optionally, 15 points;
(ii) Anti-topoisomerase I (anti-Scl70) autoantibody positive;
(iii) Anti-RNA polymerase autoantibody positive;
(iv) Anti-centromere autoantibody negative; and
(v) Disease duration ≤7 years.

In a further aspect, there is provided a BlyS antagonist for use in the treatment of SSc, for example, SSc-ILD, for example, with progressive SSc-ILD, in a human subject, wherein the human subject is characterised by:
(i) Presence of thickened skin with a modified Rodnan Skin Score ≥10 points, optionally, ≥15 points;
(ii) Disease duration ≤7 years; and
(iii) Anti-RNA polymerase autoantibody positive.

In a further aspect, there is provided a BlyS antagonist for use in the treatment of SSc, for example, SSc-ILD, for example, with progressive SSc-ILD, in a human subject, wherein the human subject is characterised by:
(i) Presence of thickened skin with a modified Rodnan Skin Score 10 points, optionally, ≥15 points;
(ii) Disease duration ≤7 years; and
(iii) Anti-centromere polymerase autoantibody negative.

In one aspect, there is provided a BlyS antagonist for use in the treatment of a subject diagnosed with SSc, for example, SSc-ILD with evidence of progression, wherein the human subject is characterised by:
(i) Presence of thickened skin with a modified Rodnan Skin Score ≥10 points, optionally, ≥15 points; and
(ii) Anti-topoisomerase I (anti-Scl70) autoantibody positive.

As ILD is most progressive in the early years of disease, subjects with a diagnosis of SSc-ILD with a disease duration of ≥2 years can be screened for additional measures to determine the presence of early progressive SSc-ILD. These measures include decline in pulmonary function tests or increased extent of ILD on HRCT. Subjects who are ATA positive and have high skin scores are at greater risk of progression.

Thus, in one aspect, there is provided a BlyS antagonist for use in the treatment of a human subject diagnosed with SSc-ILD, wherein the human subject has a disease duration of 2 years and one or more of:
(i) Reduction in forced vital capacity ≥5% relative to baseline; and
(ii) a worsening of respiratory symptoms.

Thus, in one aspect, there is provided a BlyS antagonist for use in the treatment of a human subject diagnosed with SSc-ILD, wherein the human subject has a disease duration of ≥2 years and one or more of:
(i) Reduction in forced vital capacity ≥10% relative to baseline;
(ii) A reduction in forced vital capacity ≥5% relative to baseline and a reduction in carbon monoxide diffusing capacity of ≥15% relative to baseline;
(iii) Anti-topoisomerase I (anti-Scl70) autoantibody positive;
(iv) Increased extent of ILD on HRCT since previous scan; and
(v) mRSS score ≥10 points, optionally, ≥15 points.

In another aspect, there is provided a BlyS antagonist for use in the treatment of a human subject diagnosed with SSc-ILD, wherein the human subject has a disease duration of ≥2 years and one or more of:
(i) A predicted forced vital capacity <80% relative to baseline;
(ii) A relative decline in predicted forced vital capacity 10% relative to baseline;
(iii) Anti-topoisomerase I (anti-Scl70) autoantibody positive;
(iv) Increased extent of ILD on HRCT since previous scan; and
(v) mRSS score 10 points, optionally, 15 points.

In one aspect, there is provided a BlyS antagonist for use in the treatment of a human subject diagnosed with SSc-ILD, wherein the human subject has a disease duration of 2 years and shows increased extent of ILD on HRCT. In one aspect, the extent of ILD on HRCT is a risk factor for progression and an increase is in comparison to baseline, e.g., an increase since the previous scan.

In a further aspect, there is provided a BlyS antagonist for use in the treatment of a human subject diagnosed with SSc-ILD, wherein the human subject has a disease duration of 2 years and a:
(i) Reduction in forced vital capacity ≥10% relative to baseline;
(ii) A reduction in forced vital capacity 5% relative to baseline and a reduction in carbon monoxide diffusing capacity of 15% relative to baseline;
(iii) Anti-topoisomerase I (anti-Scl70) autoantibody positive; and
(iv) Increased extent of ILD on HRCT.

Previous therapies have had limited success in treating both lung and skin manifestations of Systemic sclerosis. In one aspect, therefore, there is provided an-anti-BlyS antibody for use in the treatment of Systemic sclerosis, wherein both lung and skin are improved relative to baseline.

In another embodiment, the mRSS score is ≥1, ≥2, ≥3, ≥4, ≥5, ≥6, ≥7, 8, ≥9, ≥10, ≥11, ≥12, ≥13, ≥14, or ≥15 points.

It is an object of the present invention to treat the symptoms of Systemic sclerosis. In one aspect, the treatment of Systemic sclerosis, for example, SSc-ILD is achieved by the reduction of disease progression. It is an object of the present invention to treat the symptoms of connective tissue disease. In one aspect, the treatment of connective tissue disease, for example, CTD-ILD, is achieved by the reduction of disease progression.

In one aspect, the reduction in the disease progression is characterised by a reduction in forced vital capacity of <10% relative to baseline. In one embodiment, the reduction in the disease progression is characterised by a reduction in forced vital capacity of no more than 9%, no more than 8%, no more than 7%, no more than 6%, no more than 5%, no more than 4%, no more than 3%, no more than 2%, or no more than 1% relative to baseline. For example, in one embodiment, the reduction in the disease progression is characterised by a reduction in forced vital capacity of 0-<10%, 0-9%, 1-9%, 0-8%, 1-8%, 0-70%, 1-7%, 0-6%, 1-66%, 0-5%, 1-5%, 0-4%, 1-4%, 0-3%, 1-3%, 0-2%, 1-2%, or 0-1% relative to baseline.

For example, and not limitation, in one embodiment, the baseline represents 100% (e.g., an FVC of 100 mL). In one embodiment, the disease progression is any number that is smaller than the baseline. In one embodiment, the disease progression is a decrease to <90% of baseline (e.g., an FVC of <90 mL), which represents a decrease of ≥10% relative to the baseline. In one embodiment, a reduction in disease progression (e.g., due to treatment) results in a decrease to ≥90% of baseline (e.g., an FVC of ≥90 mL), which represents decrease of ≤10% relative to the baseline.

In one embodiment, the reduction in the disease progression is characterised by a forced vital capacity greater than the baseline.

In one aspect, the reduction in the disease progression is characterised by an absolute decline in FVC ≥5% predicted as determined by a comparison of the screening lung function test and a previous lung function test done within 12 months prior to screening and worsening respiratory symptoms, or an absolute decline in DLco (corrected for Hb) ≥10% predicted as determined by a comparison of the screening lung function test and a previous lung function test done within 12 months prior to screening and worsening respiratory symptoms.

In an alternative aspect, the reduction in the disease progression is characterised by a reduction in carbon monoxide diffusing capacity (DLco) of <15% relative to baseline. In one embodiment, the reduction in the disease progression is characterised by a reduction in carbon monoxide diffusing capacity (DLco) of no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1% relative to baseline. For example, in one embodiment, the reduction in the disease progression is characterised by a reduction in carbon monoxide diffusing capacity of 0-<15%, 0-14%, 0-13%, 0-12%, 0-11%, 0-10%, 0-9%, 0-8%, 0-7%, 0-6%, 0-5%, 0-4%, 0-3%, 0-2%, or 0-1% relative to baseline.

For example, and not limitation, in one embodiment, the baseline represents 100%. In one embodiment, the disease progression is any number that is smaller than the baseline. In one embodiment, the disease progression is a decrease to ≤85% of baseline, which represents a decrease of ≥15% relative to the baseline. In one embodiment, a reduction in disease progression (e.g., due to treatment) results in a decrease to ≥85% of baseline, which represents decrease of <15% relative to the baseline.

In one embodiment, the reduction in the disease progression is characterised by a DLco greater than the baseline.

In one aspect, there is provided a BlyS antagonist for use in treating Systemic sclerosis or connective tissue disease, for example, SSc-ILD or CTD-ILD, wherein reduction in disease progression is characterised by:
i. A reduction in forced vital capacity of no more than 5%, or no more than 4%, or no more than 3%, or no more than 2%, or no more than 1% relative to baseline; and
ii. a reduction in carbon monoxide diffusing capacity of no more than 14%, or no more than 13%, or no more than 12%, or no more than 11%, or no more than 10%, or no more than 9%, or no more than 8%, or no more than 7%, or no more than 6%, or no more than 5%, or no more than 4%, or no more than 3%, or no more than 2%, or no more than 1% relative to baseline.

In one aspect, there is provided a BlyS antagonist for use in treating Systemic sclerosis, wherein the reduction in the disease progression is further characterised by an increase in modified Rodnan skin score of <20% relative to baseline. In one embodiment, there is provided a BlyS antagonist for use in treating Systemic sclerosis, wherein the reduction in the disease progression is further characterised by an increase in modified Rodnan skin score of no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1% relative to baseline. For example, in one embodiment, the reduction in the disease progression is further characterised by an increase in modified Rodnan skin score of 0-<20%, 0-19%, 0-18%, 0-17%, 0-16%, 0-15%, 0-14%, 0-13%, 0-12%, 0-11%, 0-10%, 0-9%, 0-8%, 0-7%, 0-6%, 0-5%, 0-4%, 0-3%, 0-2%, or 0-1% relative to baseline.

For example, and not limitation, in one embodiment, the baseline represents 100% (e.g., an mRSS of 20 points). In one embodiment, the disease progression is any number that is larger than the baseline. In one embodiment, the disease progression is an increase to ≥120% of baseline (e.g., an mRSS of ≥24 points), which represents an increase of 20% relative to the baseline. In one embodiment, a reduction in the disease progression (e.g., due to treatment) results in a decrease to <120% of baseline (e.g., an mRSS of <24 points), which represents an increase of <20% relative to the baseline. In one embodiment, the treatment results in a reduction in the disease progression to <100% of baseline (e.g., an mRSS of <20 points).

Although some therapies have achieved the reduction or cessation of disease progression on certain aspects of disease it is also an object of the present invention to achieve a clinically meaningful improvement in measures as a means of treatment.

In a further aspect, the treatment of Systemic sclerosis, for example, SSc-ILD, is achieved by an improvement of clinical measures. In a further aspect, the treatment of connective tissue disease, for example, CTD-ILD, is achieved by an improvement of clinical measures.

In one embodiment, the clinical measure is any one or more of: forced vital capacity (FVC), FVC % predicted, diffusing capacity of the lung for carbon monoxide (DLco), DLco % predicted, and/or modified Rodnan skin score. In a further embodiment, the clinical measure further comprises any one or more of: extent of ILD and/or corticosteroid use.

In one aspect, there is provided a BlyS antagonist for use in treating Systemic sclerosis, for example, SSc-ILD, wherein treatment is characterised by an improvement in forced vital capacity relative to baseline. In one aspect, there is provided a BlyS antagonist for use in treating connective tissue disease, for example, CTD-ILD, wherein treatment is characterised by an improvement in forced vital capacity relative to baseline. In one aspect, the improvement in forced vital capacity is any improvement such as in 0.1-5% relative to baseline or, for example, ≥5% relative to baseline, for example, the improvement in forced vital capacity is between 5-30%, 5-25%, 5-20%, 5-15%, or 5-10% relative to baseline.

Forced vital capacity may also be measured as an absolute value in mL.

In one aspect, the improvement in forced vital capacity is more than 5 mL, or more than 10 mL, or more than 15 mL, or more than 20 mL, or more than 30 mL, or more than 50 mL, or more than 100 mL, or more than 150 mL, or more than 200 mL relative to baseline after 52 weeks following start of treatment with the anti-BlyS antagonist.

In one embodiment, treatment is characterised by a stabilisation in forced vital capacity. In one embodiment, the stabilisation in forced vital capacity is a less than 0.1%, a less than 0.5%, a less than 1%, or a less than 2% change relative to baseline.

In one aspect, there is provided a BlyS antagonist for use in treating Systemic sclerosis, for example, SSc-ILD, wherein treatment is characterised by an improvement in carbon monoxide diffusing capacity (DLco) relative to baseline. In one aspect, there is provided a BlyS antagonist for use in treating connective tissue disease, for example, CTD-ILD, wherein treatment is characterised by an improvement in carbon monoxide diffusing capacity (DLco) relative to baseline. In one aspect, the improvement in carbon monoxide diffusing capacity is between 5-10%, or 5-15%, or 5-20%, or 5-25%, or 5-30%, or 5-35%, or 5-40%, or 5-45%, or 5-50% relative to baseline. For example, in one aspect, the improvement in carbon monoxide diffusing capacity is ≥25% relative to baseline.

In one embodiment, there is provided a BlyS antagonist for use in treating CTD-ILD, wherein treatment is characterised by an improvement in FVC % predicted and/or DLco % predicted relative to baseline. In one embodiment, the improvement in FVC % predicted and/or DLco % predicted is an increase in the % predicted value. In one embodiment, the improvement in FVC % predicted and/or DLco % predicted is between 5-10%, or 5-15%, or 5-20%, or 5-25%, or 5-30%, or 5-35%, or 5-40%, or 5-45%, or 5-50% relative to baseline. In a further embodiment, the improvement in FVC % predicted is such that the FVC % predicted value is ≥80%, ≥90%, or ≥100%. In a further embodiment, the improvement in DLco % predicted is such that the DLco % predicted value is ≥75%, ≥80%, ≥90%, or ≥100%.

In one aspect, there is provided a BlyS antagonist for use in treating Systemic sclerosis, for example, SSc-ILD, wherein treatment is characterised by an improvement in modified Rodnan skin score. In one aspect, the improvement in modified Rodnan skin score is more than 5%, more than 10%, more than 15%, more than 20%, or more than 25% relative to baseline. In one aspect, the improvement in modified Rodnan skin score is a reduction of more than 5%, more than 10%, more than 15%, more than 20%, or more than 25% relative to baseline. In one aspect, there is provided a BlyS antagonist for use in treating Systemic sclerosis, for example, SSc-ILD, wherein treatment is characterised by a decrease in modified Rodnan skin score.

In a further aspect, the decrease in modified Rodnan skin score is characterised by a decrease of ≥1, ≥2, ≥3, ≥4, ≥5, ≥6, ≥7, 8, ≥9, ≥10, ≥11, ≥12, ≥13, ≥14, ≥15, or ≥20 points relative to baseline.

In one aspect, the decrease in modified Rodnan skin score is characterised by a decrease of ≥5 points. In one aspect, the decrease in modified Rodnan skin score is characterised by a decrease of ≥10 points. In one aspect, the decrease in modified Rodnan skin score is characterised by a decrease of ≥20 points.

In one aspect, there is provided a BlyS antagonist for use in treating Systemic sclerosis or connective tissue disease, for example, SSc-ILD or CTD-ILD, wherein treatment is characterised by an improvement in forced vital capacity relative to baseline and an improvement in carbon monoxide diffusing capacity relative to baseline.

In another aspect, there is provided a BlyS antagonist for use in treating Systemic sclerosis, for example, SSc-ILD, wherein treatment is characterised by an improvement in forced vital capacity relative to baseline and improvement in modified Rodnan skin score relative to baseline.

In another aspect, there is provided a BlyS antagonist for use in treating Systemic sclerosis, for example, SSc-ILD, wherein treatment is characterised by an improvement in modified Rodnan skin score relative to baseline and improvement in carbon monoxide diffusing capacity relative to baseline.

In one embodiment, any one of the aforementioned improvements (for example, in FVC, FVC % predicted, DLco, DLco % predicted and/or modified Rodnan skin score) is achieved after at least 8, at least 10, at least 12, at least 20, at least 30, at least 40, at least 50, at least 51, at least 52, at least 53, at least 54, at least 55, at least 56, at least 57, at least 58, at least 59, at least 60, at least 70, at least 80, at least 90, at least 100, at least 101, at least 102, at least 103, at least 104, at least 105, or at least 110 weeks following the start of treatment with a Blys antagonist. In one embodiment, the improvement is achieved after 52 weeks or after 104 weeks following the start of treatment with the BlyS antagonist.

In one aspect, there is provided a BlyS antagonist for use in treating CTD-ILD, wherein treatment is characterised by an increase in the progression free survival of a subject with CTD-ILD. In one embodiment, the progression free survival is measured as the time until CTD progression as defined above. In one embodiment, the increase in progression free survival is an increase of at least 2%, at least 5%, at least 10%, at least 15%, or at least 20% as compared with a reference. In one embodiment, the reference is an average progression free survival time of a population of subjects who are not receiving a BlyS antagonist.

In one aspect, there is provided a BlyS antagonist for use in treating Systemic sclerosis, for example, SSc-ILD, wherein treatment is characterised by an improvement in:
Relative decline in FVC (mL) ≥10%, OR
Relative decline in FVC ≥5% to <10% and DLco decline ≥15%, OR
≥20% increase in mRSS, OR
New scleroderma renal crisis, OR
New onset of pulmonary arterial hypertension on right heart catheterization, requiring treatment, OR
New onset of left ventricular failure, defined as left ventricular ejection fraction ≤45%, requiring treatment, OR
Gastrointestinal dysmotility, requiring enteral or parenteral nutrition, OR
Ischemic digital ulcer, requiring hospitalization, or development of critical digital ischemia, gangrene, or amputation, OR
All-cause death (regardless of association with SSc).

In one aspect, there is provided a BlyS antagonist for use in the treatment of CTD-ILD, wherein treatment is characterised by a reduction in the extent of ILD relative to baseline. In one embodiment, the extent of ILD is measured by HRCT, for example, quantitative HRCT. In one embodiment, the reduction in the extent of ILD is a ≥2%, ≥5%, ≥10%, or ≥20% decrease in the extent of ILD as assessed by HRCT in the whole lung relative to baseline.

In one embodiment, the extent of ILD is measured by a quantitative measure of interstitial lung disease (QILD) and/or a quantitative measure of lung fibrosis (QLF) score. In one embodiment, the QILD and/or QLF score is a whole lung (WL) score, for example, a QILD-WL and/or a QLF-WL score. QLF measures the extent of reticular patterns with architectural distortion (fibrosis) and the QILD score incorporates QLF, ground glass opacification, and honeycombing features. In one embodiment, quantitative analysis is conducted using a computer-aided analysis tool for fibrotic patterns based on texture measures from HRCT (Kim, et al. Clinical and experimental rheumatology vol. 28, 5 Suppl 62 (2010): S26-35; and Kim, et al. European radiology vol. 21, 12 (2011): 2455-65). In one embodiment, the reduction in the extent of ILD is measured by a ≥2%, ≥5%, ≥10%, or ≥20% decrease in QILD-WL and/or QLF-WL score relative to baseline.

In one aspect, there is provided a BlyS antagonist for use in the treatment of CTD-ILD, wherein treatment is characterised by a reduction in corticosteroid use. In one embodiment, the reduction in corticosteroid use is measured by a reduction in the cumulative dose of corticosteroids over a defined time period. In one embodiment, the defined time period is 10, 20, 30, 40, 50, 51, 52, 53, 54, 55, 56, 57, 58, 59, 60, 70, 80, 90, or 100, 101, 102, 103, 104, 105, or 110 weeks from the start of treatment with the BlyS antagonist. In one embodiment, the defined time period is 52 weeks or 104 weeks from the start of treatment with the BlyS antagonist. In one embodiment, the reduction in the cumulative dose of corticosteroid is a ≥2%, ≥5%, ≥10%, ≥15%, ≥20%, ≥25%, ≥30%, ≥40%, or ≥50% reduction.

In one embodiment, the reduction in corticosteroid use is measured by a reduction in the daily dose of a corticosteroid administered to the subject. In one embodiment, the daily dose of the corticosteroid administered to the subject is reduced to ≤20 mg/day, ≤15 mg/day, ≤10 mg/day, ≤7.5 mg/day or ≤5 mg/day of the corticosteroid. In one embodiment, the corticosteroid is an oral corticosteroid. In one embodiment, the oral corticosteroid is prednisolone or equivalent.

In one embodiment, the aforementioned reduction in the extent of ILD and/or the reduction in corticosteroid use is achieved after at least 8, at least 10, at least 12, at least 20, at least 30, at least 40, at least 50, at least 51, at least 52, at least 53, at least 54, at least 55, at least 56, at least 57, at least 58, at least 59, at least 60, at least 70, at least 80, at least 90, at least 100, at least 101, at least 102, at least 103, at least 104, at least 105, or at least 110 weeks following the start of treatment with a Blys antagonist. In one embodiment, the reduction in the extent of ILD and/or the reduction in corticosteroid use is achieved after 52 weeks or after 104 weeks following the start of treatment with the BlyS antagonist. In another embodiment, the aforementioned reduction in the extent of ILD is achieved after at least 3, at least 6, at least 12, at least 15, at least 18, at least 21, at least 24 months following the start of treatment with the Blys antagonist. In one embodiment, the reduction in the extent of ILD is achieved after at least 12 months or at least 24 months following the start of treatment with the Blys antagonist.

Although treatments for Systemic sclerosis may improve aspects of disease, many subjects do not see improvements to their wellbeing and continue to suffer with fatigue, shortness of breath (dyspnoea), and physical limitations imposed by skin tightness/thickness.

The “Canadian Scleroderma Patient Survey of Health Concerns and Research Priorities” suggests that patients with SSc are more unsatisfied with healthcare than other chronically ill patients, because SSc involves more visible physical disfigurement that tends to worsen over time, contributing to high psychological morbidity (more depressive symptoms and anxiety), regular use of healthcare and related increased costs. Physicians may ignore or use unreliable measurements to evaluate psychological distress. While physicians may prioritize objective indicators of disease status, patients may perceive other aspects of their disease experience as more debilitating or distressing, like limitations in mobility and hand function, pain, fatigue, sleep disturbance, depression, sexual dysfunction, and body image distress from disfiguring changes in appearance (pigment changes, hand contractures, and facial telangiectasias, for example).

As such, in one aspect, there is provided a BlyS antagonist for use in improving the Quality of life of a human subject suffering from Systemic sclerosis, for example, Systemic sclerosis associated-ILD. In a further aspect, there is provided a BlyS antagonist for use in improving the symptom severity of a human subject suffering from Systemic sclerosis, for example, Systemic sclerosis associated-ILD. In a further aspect, there is provided a BlyS antagonist for use in the treatment of Systemic sclerosis, for example, Systemic sclerosis associated-ILD, wherein the treatment is characterised by an improvement in Quality of life and/or symptom severity. In a further aspect, the improvement in Quality of life and/or the improvement in symptom severity is characterised by a reduction in one or more measures selected from the group:
i. Fatigue, optionally, as assessed by the functional assessment of chronic illness therapy score compared to baseline;
ii. Health Assessment Questionnaire Disability Index score compared to baseline;
iii. Dyspnoea;
iv. Scleroderma Health Assessment Questionnaire;
v. SSc-associated symptoms assessment;
vi. Patient Health Questionnaire—9;
vii. The Patient-Reported Outcomes Measurement information System (PROMIS) framework;
viii. The University of California Los Angeles Sclerodermia Clinical Trial Consortium Gastrointestinal Scale 2; and/or
ix. Scleroderma skin patient-reported outcome score.

In a further aspect, the improvement in Quality of life and/or the improvement in symptom severity is characterised by an increase in one or more measures selected from the group:
i. Transition dyspnoea index score; and/or
ii. Medical outcome study form (SF-36) score.

Other measures for assessing Quality of life and/or symptom severity are known to those skilled in the art.

In one embodiment, the Patient Health Questionnaire—9 is as reported in Kroenke K, et al. J Gen Intern Med. 2001 September; 16(9):606-13. In one embodiment, the PROMIS framework is as reported at www.healthmeasures.net/explore-measurement-systems/promis (last accessed May 14, 2024). In one embodiment, the University of California Los Angeles Scleroderma Clinical Trial Consortium Gastrointestinal Scale 2 is as reported in Khanna D, et al. Arthritis Rheum. 2009 Sep. 15; 61(9):1257-63. Further detail on other of the abovementioned measures is provided below.

Patients with CTD-ILD are also affected by general degradation of health-related quality of life and by symptoms such as dyspnea, cough, and fatigue, which are common symptoms raised by patients. Thus, in one aspect, there is provided a BlyS antagonist for use in improving the Quality of life of a human subject suffering from CTD, for example, CTD-ILD. In a further aspect, there is provided a BlyS antagonist for use in improving the symptom severity of a human subject suffering from CTD, for example, CTD-ILD. In a further aspect, there is provided a BlyS antagonist for use in the treatment of CTD, for example, CTD-ILD, wherein the treatment is characterised by an improvement in Quality of life and/or symptom severity. In one embodiment, the CTD symptom is a CTD-ILD symptom. In one embodiment, the CTD-ILD symptom is any one or more of fatigue, dyspnea and/or cough. Therefore, in one embodiment, an improvement in CTD-ILD symptom severity is a reduction in fatigue, a reduction in dyspnea and/or a reduction in cough as experienced by the patient.

Fatigue is an important symptom affecting subjects with SSc and is consistently reported to be one of the most problematic and disabling aspects of the disease. Fatigue is also a common symptom raised by patients with CTD-ILD. Functional Assessment of Chronic Illness Therapy (FACIT)-Fatigue is a validated subject-reported measure developed originally to assess fatigue in individuals with cancer and has subsequently been used and validated in numerous chronic conditions, including SSc, and other chronic autoimmune conditions, such as SLE. It is a 13-item questionnaire that assesses self-reported fatigue and its impact upon daily activities and function, with a maximum score of 52 (higher scores indicate less fatigue). In one embodiment, the FACIT-Fatigue questionnaire is version 4.

Thus, in one aspect, the improvement in subject Quality of life and/or the improvement in CTD-ILD symptom severity, is measured by an increase in FACIT-Fatigue score from baseline, for example, after 52 weeks of treatment. In one such aspect, the improvement in Quality of life and/or the improvement in CTD-ILD symptom severity, is measured by an increase in FACIT-Fatigue score from baseline of ≥2, for example, ≥3 or, for example, ≥5.

In one aspect, the improvement in subject Quality of life and/or the improvement in CTD-ILD symptom severity is measured using a Living with Idiopathic Pulmonary Fibrosis (L-PF) questionnaire. In one embodiment, the L-PF questionnaire is as reported in Swigris J, et al. ERJ Open Res. 2021 May 24; 7(2):00145-2020. The L-PF questionnaire was developed with input from patients with idiopathic pulmonary fibrosis (IPF) to assess symptoms and health-related quality of life (HRQoL). It consists of 44 items divided into two modules: Symptoms (23 items) and Impacts (21 items). The Symptoms module assesses shortness of breath (dyspnea), cough, and fatigue in the past 24 hours. The Impacts module assesses multiple aspects of health-related quality of life with a recall period of 1 week. Items in both modules have response options on a five-option numeric rating score with ‘n’ anchor of 0 “Not at all” to 4 “Extremely”. Overall scores range from 0 to 100, with higher numbers indicating a greater impairment and poorer HRQoL.

In one embodiment, an improvement in CTD-ILD symptom severity is measured by a decrease in the L-PF Symptoms score relative to baseline. In one embodiment, the improvement in CTD-ILD symptom severity is measured by a decrease in the L-PF Symptoms score of at least 1, at least 2, at least 5, at least 10, at least 15, at least 20, or at least 25 points relative to baseline.

In one embodiment, the improvement in CTD-ILD symptom severity is a reduction in fatigue. In one embodiment, the reduction in fatigue is measured by a decrease in the L-PF Symptoms score within the fatigue domain of the questionnaire, relative to baseline. In one embodiment, the improvement in CTD-ILD symptom severity is a reduction in dyspnea (shortness of breath). In one embodiment, the reduction in dyspnea is measured by a decrease in the L-PF Symptoms score within the dyspnea domain of the questionnaire, relative to baseline. In one embodiment, the improvement in CTD-ILD symptom severity is a reduction in cough. In one embodiment, the reduction in cough is measured by a decrease in the L-PF Symptoms score within the cough domain of the questionnaire, relative to baseline.

In one embodiment, the improvement in subject Quality of life is measured using the L-PF Impacts score as defined above. In one embodiment, the improvement in the subject Quality of life is measured by a decrease in the L-PF Impacts score relative to baseline. In one embodiment, the decrease in the L-PF Impacts score is a decrease of at least 1, at least 2, at least 5, at least 10, at least 15, at least 20, or at least 25 points relative to baseline.

In a further embodiment, the improvement in subject Quality of life and the improvement in CTD-ILD symptom severity is measured by a decrease in the total L-PF score relative to baseline. In one embodiment, the improvement in subject Quality of life and the improvement in CTD-ILD symptom severity is measured by a decrease in the total L-PF score of at least 1, at least 2, at least 5, at least 10, at least 15, at least 20, at least 25, at least 30, at least 40, or at least 50 points relative to baseline.

Dyspnea is commonly reported as having significant impact on the daily life of SSc subjects with ILD and on CTD-ILD patients more broadly. The Mahler baseline (or Baseline Dyspnea Index) and transitional dyspnea index (BDI/TDI) assesses the severity of dyspnea at baseline while TDI denotes changes from that baseline. The instrument assesses the functional impairment, magnitude of task, and magnitude of effort.

The BDI includes five grades of severity from zero (very severe impairment) to four (no impairment) and the categories are summed to create the focal score (zero to twelve). The TDI ranges from minus three (major deterioration) to plus three (major improvement) including a zero score to indicate “no change”. For the TDI the three categories are added to obtain a focal score ranging from minus nine, including zero, to plus nine. In one embodiment, the TDI is as reported in Mahler D A, et al. Chest. 1984 June; 85(6):751-8. In a further aspect, the improvement in Quality of life, and/or the improvement in CTD-ILD symptom severity of a human subject involves a decrease in patient reported dyspnoea characterised by a TDI score ≥0. In a further aspect, the improvement in Quality of life and/or the improvement in CTD-ILD symptom severity of a human subject involves a decrease in patient reported dyspnoea characterised by a TDI score >1. That is to say, in one embodiment, the improvement in Quality of life and/or the improvement in CTD-ILD symptom severity of a human subject is characterised by an increase in a TDI score.

In a further aspect, the improvement in Quality of life and/or the improvement in symptom severity of a human subject with SSc involves improvement in the skin of the patient, as measured by a decrease in Scleroderma skin patient-reported outcome (SSPRO) score from baseline. In one embodiment, the SSPRO is as reported in Man A, et al. Ann Rheum Dis. 2017 August; 76(8):1374-1380. In one embodiment, the improvement in the skin of the patient is measured by a decrease in the SSPRO score of at least 2, at least 5, at least 10, at least 15, at least 20, at least 21, at least 22, at least 23, at least 24, at least 25, at least 30 or at least 40 points relative to baseline. In one embodiment, the SSPRO score is a SSPRO total score.

The Health Assessment Questionnaire Disability Index (HAQ-DI) is a 26-question instrument assessing the degree of difficulty a person has in accomplishing tasks in eight functional areas: dressing and grooming, arising, eating, walking, hygiene, reach, grip, and common daily activities.

In a further aspect, the improvement in Quality of life of a human subject involves improvement in the ability of the subject to achieve perform daily functions, as measured by a decrease in health assessment questionnaire disability index score from baseline.

In a further aspect, the improvement in Quality of life is measured by a decrease in health assessment questionnaire disability index score of ≥0.1, for example, ≥0.14 from baseline.

In one embodiment, the HAQ-DI is version “STANFORD-RA (MAY99—Phase 31)—English, USA”. In one embodiment, the Scleroderma Health Assessment Questionnaire is as reported in Steen V D, Medsger T A Jr. Arthritis Rheum. 1997 November; 40(11):1984-91.

The health-related quality of life of a subject can be assessed using the participant-completed medical outcome study 36-item short form (MOS SF-36) which is a generic health survey that contains 36 questions covering eight domains of health. These domains include limitations in physical activities because of health problems; limitations in social activities because of physical or emotional problems; limitations in usual role activities because of physical health problems; bodily pain; general mental health (psychological distress and well-being); limitations in usual role activities because of emotional problems; vitality (energy and fatigue); and general health perceptions. The SF-36 yields an 8-scale profile of functional health and well-being scores as well as physical and mental component health summary scores. Each scale is directly transformed into a 0-100 scale on the assumption that each question carries equal weight. The lower the score, the more disability. In one embodiment, the participant completed medical outcome study 36-item short form is a participant-completed Short-Form Health Survey 36-item version 2 (SF-36v2).

In a further aspect, the improvement in Quality of life of a human subject is measured by an increase in medical outcome study 36-item short form score from baseline. In one embodiment, the improvement in quality of life a human subject is measured by an increase in SF-36v2 score from baseline. In a further aspect, the improvement in Quality of life of a human subject is measured by an increase in medical outcome study score (such as SF-36v2 score) of ≥2 in any domain. In one embodiment, the improvement in Quality of life of a human subject is measured by an increase in SF-36v2 score of at least 1, at least 2, at least 5, at least 10, at least 15, at least 20, at least 25, at least 30, at least 40, or at least 50 points relative to baseline.

In one embodiment, the improvement in Quality of life of a human subject is measured by the King's Brief Interstitial Lung Disease (K-BILD) Questionnaire. In one embodiment, the K-BILD Questionnaire is as reported in Patel A S, et al. Thorax. 2012 September; 67(9):804-10. The K-BILD questionnaire is a patient-reported outcome (PRO) measure developed and validated for a range of ILDs. It was designed to measure the impact of ILD on the patient's wellbeing and daily life. K-BILD includes 15 items in 3 domains (Breathlessness and activity: 4 items, Psychological: 7 items, Chest symptoms: 3 items) and a single item related to finance. Participants are to rate the impact of ILD disease on various aspects of their life during the past 2 weeks using 7-point rating scale (e.g., “All of the time”, “Most of the time”, “A good bit of the time”, “Some of the time”, “A little of the time”, “Hardly any of the time”, and “None of the time”). A total score and 3 domain scores are calculated ranging from 0-100 with greater scores denoting better quality of life. In one embodiment, the improvement in Quality of life of a human subject is measured by an increase in the K-BILD questionnaire score relative to baseline. In one embodiment, the increase in the K-BILD questionnaire score is an increase of at least 1, at least 2, at least 5, at least 10, at least 15, at least 20, at least 25, at least 30, at least 40, or at least 50 points relative to baseline.

In one embodiment, the improvement in Quality of life and/or the improvement in CTD-ILD symptom severity of a human subject is measured by a physician global assessment (PhGA) score. In a further embodiment, the PhGA score is used as a measure of the overall disease severity of the human subject suffering from CTD-ILD. The PhGA score provides an overall measure of the participant's current disease activity, on a scale from 0 to 10, where a higher score indicates a greater disease severity. Thus, in one embodiment, the improvement in Quality of life, the improvement in CTD-ILD symptom severity, and/or the improvement in the overall disease severity of a human subject is measured by a decrease in a PhGA score relate to baseline. In one embodiment, the improvement in Quality of life, the improvement in CTD-ILD symptom severity, and/or the improvement in the overall disease severity is measured by a decrease in the PhGA score from baseline of at least 1, at least 2, at least 3, at least 4, at least 5, at least 6, at least 7, at least 8, or at least 9 points.

In one embodiment, the improvement in Quality of life and/or the improvement in CTD-ILD symptom severity of a human subject is measured by a Global Impression of Change and Meaningfulness of Change (PGIC)-ILD questionnaire. In a further embodiment, the PGIC-ILD questionnaire is used as a measure of the overall disease severity of the human subject suffering from CTD-ILD. The PGIC contains two items, a global question asking participants to rate their overall change in ILD severity since first starting this study, and a yes/no question asking participants to indicate whether the change is meaningful from their perspective. Participants complete the PGIC using a 7-point verbal rating scale from “Very much worse”, “Moderately worse”, “Minimally worse”, “No change”, “Minimally improved”, “Moderately improved”, to “Very much improved”. Participants then will complete “Yes” or “No” regarding whether the change that they experienced was meaningful to them or not. In one embodiment, the improvement in Quality of life, the improvement in CTD-ILD symptom severity, and/or the improvement in the overall disease severity of a human subject is measured as a positive change as recording using the PGIC-ILD questionnaire, for example, the participant rates their overall change in ILD severity as “Minimally improved”, “Moderately improved”, or “Very much improved”. In one embodiment, the aforementioned positive change is considered meaningful from their perspective.

In one aspect, any one of the aforementioned improvement in Quality of life, improvement in symptom severity, for example, CTD-ILD symptom severity, and/or improvement in overall disease severity is achieved after at least 8, at least 10, at least 12, at least 20, at least 30, at least 40, at least 50, at least 51, at least 52, at least 53, at least 54, at least 55, at least 56, at least 57, at least 58, at least 59, at least 60, at least 70, at least 80, at least 90, at least 100, at least 101, at least 102, at least 103, at least 104, at least 105, or at least 110 weeks following the start of treatment with the Blys antagonist. In one embodiment, the improvement is achieved after 52 weeks or after 104 weeks following the start of treatment with the BlyS antagonist.

In some embodiments, the aforementioned Quality of life is a health-related quality of life (HRQoL).

With regard to SSc, current treatment approaches, with some variability between regions, generally include the off-label use of methotrexate (for skin thickening only) and mycophenolate as first line therapies. Cyclophosphamide can be used as a first line therapy but given mycophenolate offers a better benefit-risk profile, common clinical practice is to reserve cyclophosphamide and also rituximab for subjects not responding to mycophenolate, while azathioprine is used as maintenance after cyclophosphamide treatment or as an alternative to mycophenolate. Current expert recommendations for treatment of SSc suggest that all subjects with clinical ILD, as well as those with subclinical ILD, but at high risk of progression, should be considered for immunomodulatory treatment, with mycophenolate being the most broadly used first line treatment. As such, permitted immunosuppressive treatments for SSc include mycophenolate (mycophenolate mofetil or mycophenolate sodium), methotrexate, or azathioprine, not given in combination.

In one aspect, the BlyS antagonist, such as an anti-BlyS antibody, for example, belimumab, for use in the treatment of Systemic sclerosis is administered as a monotherapy; in another aspect, it is administered in combination with other therapies. In one aspect, the BlyS antagonist, such as an anti-BlyS antibody, for example, belimumab, for use in the treatment of CTD-ILD, is administered as a monotherapy; in another aspect, it is administered in combination with a further therapy. In one embodiment, the BlyS antagonist is administered simultaneously, separately, or sequentially with the further therapy. Thus, in one aspect, the anti-BlyS antagonist may be administered with additional immunosuppressive agents, for example, with Cyclophosphamide (CYC), Azathioprine (AZA), Methotrexate (MTX), Mycophenolate Mofetil (MMF), and/or Mycophenolate sodium.

In one aspect, the BlyS antagonist for use in the treatment of CTD-ILD is administered with an additional immunosuppressive agent. In one embodiment, the additional immunosuppressive agent is one or more of mycophenolate mofetil, mycophenolate sodium, methotrexate, azathioprine, a corticosteroid, a calcineurin inhibitor, a pyrimidine synthesis inhibitor, and/or an anti-malarial agent. In one embodiment, the corticosteroid is an oral corticosteroid, for example, prednisolone. In one embodiment, the calcineurin inhibitor is tacrolimus or cyclosporin. In one embodiment, the pyrimidine synthesis inhibitor is leflunomide. In one embodiment, the anti-malarial agent is hydroxychloroquine. In one embodiment, the additional immunosuppressive agent is administered at a dose in accordance with standard treatment guidelines.

In one aspect, the additional immunosuppressive agent is methotrexate. In one embodiment, the methotrexate is administered at a dose of ≤25 mg/week. In one aspect, the additional immunosuppressive agent is methotrexate, wherein the methotrexate is administered at a dose of ≤25 mg/week.

In one aspect, the additional immunosuppressive agent is azathioprine. In one embodiment, the azathioprine is administered at a dose of ≤2.5 mg/kg/day. In one aspect, the additional immunosuppressive agent is azathioprine, wherein the azathioprine is administered at a dose of ≤2.5 mg/kg/day.

In one aspect, the additional immunosuppressive agent is mycophenolate mofetil. In one embodiment, the mycophenolate mofetil is administered at a dose of ≤3000 mg/day. In one aspect, the additional immunosuppressive agent is mycophenolate mofetil, wherein the mycophenolate mofetil is administered at a dose of ≤3000 mg/day.

In one aspect, the additional immunosuppressive agent is mycophenolate sodium. In one embodiment, the mycophenolate sodium is administered at a dose of ≤2160 mg/day. In one aspect, the additional immunosuppressive agent is mycophenolate sodium, wherein the mycophenolate sodium is administered at a dose of ≤2160 mg/day.

In one aspect, the additional immunosuppressive agent is tacrolimus. In one embodiment, the tacrolimus is administered at a dose of ≤5 mg/day or ≤2.5 mg/day. In one aspect, the additional immunosuppressive agent is tacrolimus, wherein the tacrolimus is administered at a dose of ≤5 mg/day or ≤2.5 mg/day.

In one aspect, the additional immunosuppressive agent is cyclosporin. In one embodiment, the cyclosporin is administered at a dose of ≤4 mg/kg/day. In one aspect, the additional immunosuppressive agent is cyclosporin, wherein the cyclosporin is administered at a dose of ≤4 mg/kg/day.

In one aspect, the additional immunosuppressive agent is hydroxychloroquine. In one embodiment, the hydroxychloroquine is administered at a dose of ≤400 mg/day. In one aspect, the additional immunosuppressive agent is hydroxychloroquine, wherein the hydroxychloroquine is administered at a dose of ≤400 mg/day.

In one aspect, the additional immunosuppressive agent is leflunomide. In one embodiment, the leflunomide is administered at a dose of ≤20 mg/day. In one aspect, the additional immunosuppressive agent is leflunomide, wherein the leflunomide is administered at a dose of ≤20 mg/day.

In one aspect, the additional immunosuppressive agent is a corticosteroid. In one embodiment, the corticosteroid is an oral corticosteroid. In one embodiment, the oral corticosteroid is prednisolone. In one embodiment, the corticosteroid, for example, prednisolone or equivalent, is administered at a dose of ≤20 mg/day. In one aspect, the additional immunosuppressive agent is prednisolone, wherein the prednisolone is administered at a dose of ≤20 mg/day.

In an additional aspect, the further therapy is an anti-fibrotic agent. Therefore, in one embodiment, the BlyS antagonist for use in the treatment of CTD-ILD, for example, SSc-ILD, is administered with an additional anti-fibrotic agent. In one embodiment, the additional anti-fibrotic agent is nintedanib. In one embodiment, the additional anti-fibrotic agent, for example, nintedanib, is administered at a dose in accordance with standard treatment guidelines.

The term “BlyS antagonist” as used herein refers to an agent which reduces or blocks BlyS activity, for example, by binding to BlyS or the BR3, TACI, or BCMA receptors. In one embodiment, the BlyS antagonist is a small chemical molecule. In another aspect, the BlyS antagonist is an anti-BlyS binding protein or an anti-BlyS receptor binding protein. In one aspect, the BlyS antagonist binds specifically to BlyS.

In a further aspect, the BlyS antagonist is an anti-BlyS antibody. In one aspect, the anti-BlyS antibody comprises any one or more of: CDRH 1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5, and CDRL3 of SEQ ID NO: 6. In one aspect, the anti-BlyS antibody comprises CDRH1 of SEQ ID NO: 1, CDRH2 of SEQ ID NO: 2, CDRH3 of SEQ ID NO: 3, CDRL1 of SEQ ID NO: 4, CDRL2 of SEQ ID NO: 5, and CDRL3 of SEQ ID NO: 6. In one such aspect, the anti-BlyS antibody comprises a heavy chain variable region (VH) with at least 80%, or at least 85%, or at least 90%, or at least 95%, or at least 96%, or at least 97%, or at least 98%, or at least 99% identity to SEQ ID NO: 7 and/or a light chain variable region (VL) with at least 80%, or at least 85%, or at least 90%, or at least 95%, or at least 96%, or at least 97%, or at least 98%, or at least 99% identity to SEQ ID NO: 8.

In another aspect, the anti-BlyS antibody comprises at least one of a variable heavy chain sequence of SEQ ID NO: 7 and a variable light chain sequence of SEQ ID NO: 8. In one aspect, the anti-BlyS antibody comprises at least one of a variable heavy chain sequence of SEQ ID NO: 7 and a variable light chain sequence of amino acids 1 to 108 of SEQ ID NO: 8. In a further aspect, the anti-BlyS antibody comprises both the heavy chain variable region (VH) of SEQ ID NO: 7 and the light chain variable region (VL) of SEQ ID NO: 8. In a further aspect, the anti-BlyS antibody comprises both the heavy chain variable region (VH) of SEQ ID NO: 7 and the light chain variable region (VL) of amino acids 1 to 108 of SEQ ID NO: 8.

In a further aspect, the anti-BlyS antibody comprises a heavy chain region (HC) with at least 80%, or at least 85%, or at least 90%, or at least 95%, or at least 96%, or at least 97%, or at least 98%, or at least 99% identity to SEQ ID NO: 9 and/or a light chain region (LC) with at least 80%, or at least 85%, or at least 90%, or at least 95%, or at least 96%, or at least 97%, or at least 98%, or at least 99% identity to SEQ ID NO: 10.

In a further aspect, the anti-BlyS antibody comprises a heavy chain region (HC) of SEQ ID NO: 9 and a light chain region (LC) of SEQ ID NO: 10.

In a specific aspect, the anti-BlyS antibody is belimumab.

The term “antibody” is used herein in the broadest sense to refer to molecules with an immunoglobulin-like domain (for example, IgG, IgM, IgA, IgD, or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)₂, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, Vol 23, No. 9, 1126-1136).

The term, full, whole or intact antibody, used interchangeably herein, refers to a heterotetrameric glycoprotein with an approximate molecular weight of 150,000 Daltons. An intact antibody is composed of two identical heavy chains (HCs) and two identical light chains (LCs) linked by covalent disulphide bonds. This H₂L₂ structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE, and IgD are defined by distinct heavy chain amino acid sequences, which are called μ, α, γ, ε, and δ, respectively, each heavy chain can pair with either a K or λ light chain. The majority of antibodies in the serum belong to the IgG class, there are four isotypes of human IgG (IgG1, IgG2, IgG3, and IgG4), the sequences of which differ mainly in their hinge region.

Fully human antibodies can be obtained using a variety of methods, for example, using yeast-based libraries or transgenic animals (e.g., mice) that are capable of producing repertoires of human antibodies. Yeast presenting human antibodies on their surface that bind to an antigen of interest can be selected using FACS (Fluorescence-Activated Cell Sorting) based methods or by capture on beads using labelled antigens. Transgenic animals that have been modified to express human immunoglobulin genes can be immunised with an antigen of interest and antigen-specific human antibodies isolated using B-cell sorting techniques. Human antibodies produced using these techniques can then be characterised for desired properties such as affinity, developability and selectivity.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301), or an EGF domain.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4^{th} Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example, those set out in Chothia et al. (1989) Nature 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

CDRs may be modified by at least one amino acid substitution, deletion, or addition, wherein the variant antigen binding protein substantially retains the biological characteristics of the unmodified protein, such as, for example, the ability to block BlyS from binding to its receptor.

It will be appreciated that each of CDR H1, H2, H3, L1, L2, or L3 may be modified alone or in combination with any other CDR, in any permutation or combination. In one embodiment, a CDR is modified by the substitution, deletion, or addition of up to 3 amino acids, for example, 1 or 2 amino acids, for example, 1 amino acid. Typically, the modification is a substitution, particularly a conservative substitution, for example, as shown in Table 3 below.

TABLE 3

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence | Gly, Pro |
| chain orientation | |
| Aromatic | Trp, Tyr, Phe |

For example, in a variant CDR, the flanking residues that comprise the CDR as part of alternative definition(s), e.g., Kabat or Chothia, may be substituted with a conservative amino acid residue.

Such antigen binding proteins comprising variant CDRs as described above may be referred to herein as “functional CDR variants”.

“Percent identity” or “% identity” between a query amino acid sequence and a subject amino acid sequence is the “Identities” value, expressed as a percentage, that is calculated using a suitable algorithm (e.g., BLASTP, FASTA, Needleman-Wunsch, Smith-Waterman, LALIGN, or GenePAST/KERR) or software (e.g., DNASTAR Lasergene, GenomeQuest, EMBOSS needle, or EMBOSS infoalign), over the entire length of the query sequence after a pair-wise global sequence alignment has been performed using a suitable algorithm (e.g., Needleman-Wunsch or GenePAST/KERR) or software (e.g., DNASTAR Lasergene or GenePAST/KERR). Importantly, a query amino acid sequence may be described by an amino acid sequence disclosed herein, in particular, in one or more of the claims.

For antibody sequences, the % identity may be determined across the entire length of the query sequence, including the CDRs. Alternatively, the % identity may exclude one or more or all of the CDRs, for example, all of the CDRs are 100% identical to the subject sequence and the % identity variation is in the remaining portion of the query sequence, e.g., the framework sequence, so that the CDR sequences are fixed and intact. The variant sequence substantially retains the biological characteristics of the unmodified protein.

The reduction or inhibition in biological activity may be partial or total. In some aspects, the anti-BlyS antibody may neutralise the activity of BlyS by at least 20%, at least 30%, at least 40%, at least 50%, at least 55%, at least 60%, at least 65%, at least 70%, at least 75%, at least 80%, at least 82%, at least 84%, at least 86%, at least 88%, at least 90%, at least 92%, at least 94%, at least 95%, at least 96%, at least 97%, at least 98%, at least 99%, or 100% relative to BlyS activity in the absence of the anti-BlyS antibody. Neutralisation may be determined or measured using one or more assays known to the skilled person or as described herein.

An antagonist or antibody antigen binding protein as described herein may be incorporated into pharmaceutical compositions for use in the treatment of the human diseases described herein. In one embodiment, the pharmaceutical composition comprises an antigen binding protein in combination with one or more pharmaceutically acceptable carriers and/or excipients.

Such compositions comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular, intraocular, and intraportal). In one embodiment, the pharmaceutical composition may be given subcutaneously, however, some subjects may find subcutaneous administration difficult to achieve as a result of skin thickening and as such intravenous administration is also provided. Pharmaceutical compositions may be suitable for topical administration (which includes, but is not limited to, epicutaneous, inhaled, intranasal, or ocular administration) or enteral administration (which includes, but is not limited to, oral, vaginal, or rectal administration).

The pharmaceutical composition may be included in a kit containing the antigen binding protein together with other medicaments and/or with instructions for use. For convenience, the kit may comprise the reagents in predetermined amounts with instructions for use. The kit may also include devices used for administration of the pharmaceutical composition.

The terms “individual”, “subject” and “patient” are used herein interchangeably. In one embodiment, the subject is an animal. In another embodiment, the subject is a mammal, such as a primate, for example, a marmoset or monkey. In another embodiment, the subject is a human.

The antigen binding protein described herein may also be used in methods of treatment. It will be appreciated by those skilled in the art that references herein to treatment refer to the treatment of established conditions. However, compounds of the invention may, depending on the condition, also be useful in the prevention of certain diseases. The antigen binding protein described herein is used in an effective amount for therapeutic, prophylactic, or preventative treatment. A therapeutically effective amount of the antigen binding protein described herein is an amount effective to ameliorate or reduce one or more symptoms of, or to prevent, or cure the disease. The term “prevention” refers to avoidance of the stated disease in a subject who is not suffering from the stated disease.

Antigen binding proteins may be prepared by any of a number of conventional techniques. For example, antigen binding proteins may be purified from cells that naturally express them (e.g., an antibody can be purified from a hybridoma that produces it) or produced in recombinant expression systems.

A number of different expression systems and purification regimes can be used to generate the antigen binding protein of the invention. Generally, host cells are transformed with a recombinant expression vector encoding the desired antigen binding protein. The expression vector may be maintained by the host as a separate genetic element or integrated into the host chromosome depending on the expression system. A wide range of host cells can be employed, including Prokaryotes (including Gram negative or Gram positive bacteria, for example, Escherichia coli, Bacilli sp., Pseudomonas sp., Corynebacterium sp.), Eukaryotes including yeast (for example, Saccharomyces cerevisiae, Pichia pastoris), fungi (for example, Aspergillus sp.), or higher Eukaryotes including insect cells and cell lines of mammalian origin (for example, CHO, NS0, PER.C6, HEK293, HeLa).

The host cell may be an isolated host cell. The host cell is usually not part of a multicellular organism (e.g., plant or animal). The host cell may be a non-human host cell.

Appropriate cloning and expression vectors for use with bacterial, fungal, yeast, and mammalian host cells are known in the art.

The cells can be cultured under conditions that promote expression of the antigen binding protein using a variety of equipment such as shake flasks, spinner flasks, and bioreactors. The polypeptide is recovered by conventional protein purification procedures. Protein purification procedures typically consist of a series of unit operations comprised of various filtration and chromatographic processes developed to selectively concentrate and isolate the antigen binding protein. The purified antigen binding protein may be formulated in a pharmaceutically acceptable composition.

### EMBODIMENTS

The present disclosure also provides the following numbered embodiments. Combinations of features of the present disclosure presented below are exemplary, and not to be construed as exhaustive.

1. A BlyS antagonist for use in the treatment of a human subject diagnosed with systemic sclerosis.

2. A BlyS antagonist for use in the treatment of a human subject diagnosed with systemic sclerosis-associated interstitial lung disease.

3. A BlyS antagonist for use according to embodiment 1 or 2, wherein the anti-BlyS antagonist is an anti-BlyS antibody.

4. An anti-BlyS antibody for use according to embodiment 2 or 3 for the treatment of a human subject diagnosed with limited cutaneous systemic sclerosis-associated interstitial lung disease.

5. A BlyS antagonist for use according to embodiment 4, wherein the limited cutaneous systemic sclerosis is characterised via a modified Rodnan skin score of >0 points distal to the knees and/or elbows.

6. A BlyS antagonist for use according to any one of embodiments 1 to 3 for the treatment of a human subject diagnosed with diffuse cutaneous systemic sclerosis.

7. A BlyS antagonist for use according to embodiment 6, wherein the human subject is characterised by the presence of thickened skin with a modified Rodnan skin score >0 points over at least one skin area proximal to the knees and/or elbows in addition to distal areas.

8. A BlyS antagonist for the use according to any preceding embodiment, wherein the treatment is characterised by a reduction in disease progression.

9. A BlyS antagonist for the use according to embodiment 8, wherein the disease progression is further characterised by a reduction in forced vital capacity of at least 10% relative to baseline.

10. A BlyS antagonist for the use according to embodiment 8 or 9, wherein the reduction in the disease progression is characterised by a reduction in forced vital capacity of no more than 9%, no more than 8%, no more than 7%, no more than 6%, no more than 5%, no more than 4%, no more than 3%, no more than 2%, or no more than 1% relative to baseline.

11. A BlyS antagonist for the use according to embodiment 8 or 9, wherein the reduction in the disease progression is characterised by a reduction in forced vital capacity of 0.1-<10%, or 0.1-9%, or 0.1-8%, or 0.1-7%, or 0.1-6%, or 0.1-5%, or 0.1-4%, or 0.1-3%, or 0.1-2%, or 0.1% relative to baseline.

12. A BlyS antagonist for the use according to embodiment 8, wherein the disease progression is characterised by a reduction in carbon monoxide diffusing capacity of at least 15% relative to baseline.

13. A BlyS antagonist for the use according to embodiment 8 or 12, wherein the reduction in the disease progression is characterised by a reduction in carbon monoxide diffusing capacity of no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, no more than 1%, or 0% relative to baseline.

14. A BlyS antagonist for the use according to embodiment 8 or 12, wherein the reduction in the disease progression is characterised by a reduction in carbon monoxide diffusing capacity of 0.1-<15%, or 0.1-14%, or 0.1-13%, or 0.1-12%, or 0.1-11%, or 0.1-10%, or 0.1-9%, or 0.1-8%, or 0.1-7%, or 0.1-6%, or 0.1-5%, or 0.1-4%, or 0.1-3%, or 0.1-2%, or 0.1-1% relative to baseline.

15. A BlyS antagonist for use according to embodiment 8, wherein the disease progression is further characterised by:
i. a reduction in forced vital capacity of at least 5% or at least 10% relative to baseline; or
ii. a reduction in forced vital capacity of at least 5% relative to baseline and a reduction in carbon monoxide diffusing capacity of at least 15% relative to baseline.

16. A BlyS antagonist for use according to embodiment 8, wherein the reduction in the disease progression is characterised by:
i. A reduction in forced vital capacity of no more than 5%, or no more than 4%, or no more than 3%, or no more than 2%, or no more than 1% relative to baseline; and
ii. a reduction in carbon monoxide diffusing capacity of no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, no more than 1%, or 0% relative to baseline.

17. A BlyS antagonist for use according to any one of embodiments 8 to 16, wherein the disease progression is further characterised by an increase in modified Rodnan skin score of ≥20% relative to baseline.

18. A BlyS antagonist for use according to any one of embodiments 8 to 17, wherein the reduction in the disease progression is further characterised by an increase in modified Rodnan skin score of no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, no more than 1%, or 0% relative to baseline.

19. A BlyS antagonist for use according to embodiment 17, wherein the reduction in the disease progression is further characterised by an increase in modified Rodnan skin score of 0-<20%, 0-19%, 0-18%, 0-17%, 0-16%, 0-15%, 0-14%, 0-13%, 0-12%, 0-11%, 0-10%, 0-9%, 0-8%, 0-7%, 0-6%, 0-5%, 0-4%, 0-3%, 0-2%, or 0-1% relative to baseline.

20. A BlyS antagonist for use according to embodiment 8, wherein the disease progression is further characterised by:
i. A reduction in forced vital capacity ≥10% relative to baseline; and
ii. An increase in modified Rodnan skin score ≥20% relative to baseline.

21. A BlyS antagonist for use according to any previous embodiment, wherein the disease progression is further characterised by:
(i) A reduction in forced vital capacity of at least 10% relative to baseline; or
(ii) A reduction in forced vital capacity of at least 5% relative to baseline and a reduction in carbon monoxide diffusing capacity of at least 15% relative to baseline; or
(iii) A ≥20% increase in mRSS relative to baseline; or
(iv) New scleroderma renal crisis; or
(v) New onset of pulmonary arterial hypertension on right heart catheterization, requiring treatment; or
(vi) New onset of left ventricular failure, defined as left ventricular ejection fraction ≤45%, requiring treatment; or
(vii) Gastrointestinal dysmotility, requiring enteral or parenteral nutrition; or
(viii) Ischemic digital ulcer, requiring hospitalization, or development of critical digital ischemia, gangrene, or amputation; or
(ix) Any cause of death (regardless of association with SSc).

22. A BlyS antagonist for the use according to any preceding embodiment, wherein the treatment is characterised by an increase in forced vital capacity of the human subject compared to baseline.

23. A BlyS antagonist for use according to embodiment 22, wherein an improvement in the forced vital capacity of ≥5% relative to baseline is achieved in the human subject.

24. A BlyS antagonist for use according to embodiment 23, wherein the improvement in the forced vital capacity of the human subject is shown as an increase between 5-30%, 5-25%, 5-20%, 5-15%, or 5-10% relative to baseline.

25. A BlyS antagonist for use according to embodiment 22, wherein the improvement in the forced vital capacity of the human subject is more than 5 mL, more than 10 mL, more than 15 mL, more than 17 mL, more than 20 mL, more than 25 mL, more than 35 mL, more than 50 mL, more than 100 mL, or more than 200 mL relative to baseline.

26. A BlyS antagonist for the use according to any preceding embodiment, wherein the treatment is characterised by an improvement in carbon monoxide diffusing capacity relative to baseline.

27. A BlyS antagonist for use according to embodiment 26, wherein the improvement in the carbon monoxide diffusing capacity of the human subject is between 5-10%, 5-15%, 5-20%, 5-25%, 5-30%, 5-35%, 5-40%, 5-45%, or 5-50% relative to baseline.

28. A BlyS antagonist for use according to embodiment 26, wherein the improvement in carbon monoxide diffusing capacity is ≥25% relative to baseline.

29. A BlyS antagonist for use according to any preceding embodiment, wherein the treatment is characterised by an improvement in modified Rodnan skin score in the human subject.

30. A BlyS antagonist for use according to embodiment 29, wherein the improvement in the modified Rodnan skin score is a decrease of more than 5%, more than 10%, more than 15%, more than 20%, or more than 25% relative to baseline.

31. A BlyS antagonist for use according to any preceding embodiment, wherein the treatment is characterised by an improvement in forced vital capacity and an improvement in carbon monoxide diffusing capacity relative to baseline.

32. A BlyS antagonist for use according to any preceding embodiment, wherein the treatment is characterised by an improvement in forced vital capacity relative to baseline and an improvement in modified Rodnan skin score relative to baseline.

33. A BlyS antagonist for use according to any preceding embodiment, wherein the treatment is characterised by an improvement in modified Rodnan skin score relative to baseline and an improvement in carbon monoxide diffusing capacity relative to baseline.

34. A BlyS antagonist for use according to any preceding embodiment, wherein the treatment of the human subject is further characterised by:
(a) a reduction in one or more measures selected from the group of:
   i. Fatigue, optionally as assessed by the functional assessment of chronic illness therapy score compared to baseline;
   ii. Dyspnoea; and/or
   iii. Health Assessment Questionnaire Disability Index score; and/or
(b) an increase in one or more measures selected from the group of:
   v. Transition Dyspnoea Index score; and/or
   vi. Medical outcome study 36 item short form score.

35. A BlyS antagonist for use according to any preceding embodiment, wherein the human subject is anti-topoisomerase I (anti-Scl70) autoantibody positive.

36. A BlyS antagonist for use according to any preceding embodiment, wherein the human subject is anti-RNA polymerase autoantibody positive.

37. A BlyS antagonist for use according to any preceding embodiment, wherein the human subject is anti-centromere autoantibody negative.

38. A BlyS antagonist for use according to any preceding embodiment, wherein the human subject is anti-topoisomerase I (anti-Scl70) autoantibody positive, anti-RNA polymerase autoantibody positive, and anti-centromere autoantibody negative.

39. A BlyS antagonist for use according to any preceding embodiment, wherein the human subject is characterised by the presence of thickened skin with a modified Rodnan skin score ≥10 points, optionally, ≥15 points.

40. A BlyS antagonist for use according to any previous embodiment, wherein the human subject is characterised by a disease duration ≤7 years.

41. A BlyS antagonist for use according to any preceding embodiment, wherein the human subject is characterised by one or more of:
i. Presence of thickened skin with a modified Rodnan skin score ≥10 points, optionally, ≥15 points;
ii. Anti-topoisomerase I (anti-Scl70) autoantibody positive;
iii. Anti-RNA polymerase autoantibody positive;
iv. Anti-centromere autoantibody negative; and
v. Disease duration ≤7 years.

42. A BlyS antagonist for use according to embodiment 41, wherein the human subject is characterised by:
(i) Presence of thickened skin with a modified Rodnan Skin Score ≥10 points, optionally, ≥15 points;
(ii) Disease duration ≤7 years; and
(iii) Anti-topoisomerase I (anti-Scl70) autoantibody positive.

43. A BlyS antagonist for use according to embodiment 41, wherein the human subject is characterised by:
(i) Presence of thickened skin with a modified Rodnan Skin Score 10 points, optionally, 15 points;
(ii) Disease duration ≤7 years; and
(iii) Anti-RNA polymerase autoantibody positive.

44. A BlyS antagonist for use according to embodiment 41, wherein the human subject is characterised by:
(i) Presence of thickened skin with a modified Rodnan Skin Score ≥10 points, optionally, ≥15 points;
(ii) Disease duration ≤7 years; and
(iii) Anti-centromere polymerase autoantibody negative.

45. A BlyS antagonist for use according to any preceding embodiment, wherein the human subject is characterised by:
i. Presence of thickened skin with a modified Rodnan Skin Score ≥10 points, optionally, ≥15 points;
ii. Anti-topoisomerase I (anti-Scl70) autoantibody positive; and
iii. Anti-centromere autoantibody negative.

46. A BlyS antagonist for use according to any one of embodiments 40 to 45, wherein the human subject is diagnosed with lcSSc-ILD.

47. A BlyS antagonist for use according to any one of embodiments 40 to 45, wherein the human subject is diagnosed with dcSSc-ILD.

48. A BlyS antagonist for use according to any preceding embodiment, wherein the human subject has a disease duration of ≥2 years and one or more of:
i. Reduction in forced vital capacity;
ii. Reduction in carbon monoxide diffusing capacity;
iii. Anti-topoisomerase I (anti-Scl70) autoantibody positive; and iv. Increased extent of ILD on HRCT.

49. An anti-BlyS antibody for use according to any one of embodiments 3 to 48, wherein the antibody comprises: CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6 or variants thereof.

50. An anti-BlyS antibody for use according to any one of embodiments 3 to 49, wherein the antibody comprises: CRDH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6.

51. An anti-BlyS antibody for use according to any one of embodiments 3 to 50, wherein the antibody comprises a variable heavy chain sequence of SEQ ID NO: 7, or a sequence which is at least 90%, at least 91%, at least 92%, at least 93%, at least 94%, at least 95%, at least 96%, at least 97%, at least 98%, or at least 99% identical thereto, and a light chain variable sequence of SEQ ID NO: 8 or a sequence which is at least 90%, at least 91%, at least 92%, at least 93%, at least 94%, at least 95%, at least 96%, at least 97%, at least 98%, or at least 99% identical thereto.

52. An anti-BlyS antibody for use according to any one of embodiments 3 to 51, wherein the antibody comprises a variable heavy chain sequence of SEQ ID NO: 7 and a variable light chain sequence of SEQ ID NO: 8.

53. An anti-BlyS antibody for use according to any one of embodiments 3 to 52, wherein the antibody is belimumab.

54. The anti-BlyS antibody for use according to any one of embodiments 3 to 53, wherein the antibody is administered subcutaneously.

55. The anti-BlyS antibody for use according to embodiment 54, wherein the antibody is administered to a subject at a unit dose of 200 mg per week.

56. The anti-BlyS antibody for use according to embodiment 53, wherein the antibody is administered intravenously.

57. The anti-BlyS antibody for use according to embodiment 56, wherein the antibody is administered to a subject at a unit dose of 10 mg/kg.

58. The anti-BlyS antibody for use according to any one of embodiments 3 to 57, wherein the antibody is co-administered with an additional immunosuppressive agent.

59. The anti-BlyS antibody for use according to embodiment 58, wherein the additional immunosuppressive agent is selected from the group methotrexate, mycophenolate mofetil, mycophenolate sodium, or azathioprine.

60. The anti-BlyS antibody for use according to embodiment 59, wherein the additional immunosuppressive agent is methotrexate.

61. The anti-BlyS antibody for use according to embodiment 59, wherein the additional immunosuppressive agent is mycophenolate mofetil.

62. The anti-BlyS antibody for use according to embodiment 59, wherein the additional immunosuppressive agent is mycophenolate sodium.

63. The anti-BlyS antibody for use according to embodiment 59, wherein the additional immunosuppressive agent is azathioprine.

64. A BlyS antagonist according to any preceding embodiment for use in the manufacture of a medicament for the treatment of Systemic sclerosis or Systemic sclerosis-associated interstitial lung disease.

65. A method of treating Systemic sclerosis or Systemic sclerosis-associated interstitial lung disease comprising administering a BlyS antagonist according to any previous embodiment.

66. A method of improving the quality of life of a human subject suffering from early diffuse cutaneous systemic sclerosis-associated interstitial lung disease or early limited cutaneous systemic sclerosis-associated interstitial lung disease, wherein the human subject is administered a therapeutically suitable amount of an anti-BlyS antibody.

67. An anti-BlyS antibody for use in the treatment of systemic sclerosis-associated interstitial lung disease in a human subject in need thereof.

68. An anti-BlyS antibody for use according to embodiment 67, in the treatment of diffuse cutaneous systemic sclerosis-associated interstitial lung disease.

69. An anti-BlyS antibody for use according to embodiment 68, in the treatment of early diffuse cutaneous systemic sclerosis-associated interstitial lung disease.

70. An anti-BlyS antibody for use according to any one of embodiments 67 to 69, wherein the treatment is characterised by a reduction in disease progression.

71. An anti-BlyS antibody for use according to embodiment 70, wherein the disease progression is characterised by a reduction in forced vital capacity.

72. An anti-BlyS antibody for use according to embodiment 71, wherein the disease progression is further characterised by:
(i) a reduction in forced vital capacity of at least 10% relative to baseline; or
(ii) a reduction in forced vital capacity of at least 5% relative to baseline and a reduction in carbon monoxide diffusing capacity of at least 15% relative to baseline.

73. An anti-BlyS antibody for use according to embodiment 70 or 71, wherein the disease progression is further characterised by an increase in modified Rodnan skin score of ≥20% relative to baseline.

74. An anti-BlyS antibody for use according to any one of embodiments 67 to 73, wherein the treatment is characterised by an improvement in forced vital capacity relative to baseline.

75. An anti-BlyS antibody for use according to any one of embodiments 67 to 74, wherein the treatment is characterised by a reduction in modified Rodnan skin score of ≥20% relative to baseline.

76. An anti-BlyS antibody for use according to any one of embodiments 67 to 75, wherein a reduction in one or more measures relative to baseline selected from the group is achieved in the human subject:
(i) Fatigue as assessed by the functional assessment of chronic illness therapy-fatigue score compared to baseline;
(ii) Transition dyspnoea index score;
(iii) Health Assessment Questionnaire Disability Index score; or
(iv) Medical outcome study 36-item short form score.

77. An anti-BlyS antibody for use according to any one of embodiments 67 to 76, wherein the human subject is characterised by one or more of:
(i) Presence of thickened skin with a modified Rodnan Skin Score ≥15;
(ii) Anti-topoisomerase I (anti-Scl70) autoantibody positive;
(iii) Anti-RNA polymerase autoantibody positive;
(iv) Anti-centromere autoantibody negative; and
(v) Disease duration ≤7 years.

78. An anti-BlyS antibody for use according to any one of embodiments 67 to 77, wherein the human subject is anti-topoisomerase I (anti-Scl70) autoantibody positive.

79. An anti-BlyS antibody for use according to embodiment 77, wherein the human subject is characterised by:
(i) Presence of thickened skin with a modified Rodnan Skin Score ≥15;
(ii) Disease duration ≤7 years; and
(iii) Anti-topoisomerase I (anti-Scl70) autoantibody positive.

80. An anti-BlyS antibody for use according to embodiment 77, wherein the human subject is characterised by:
(i) Presence of thickened skin with a modified Rodnan Skin Score ≥15;
(ii) Disease duration ≤7 years; and
(iii) Anti-RNA polymerase autoantibody positive.

81. An anti-BlyS antibody for use according to embodiment 77, wherein the human subject is characterised by:
(i) Presence of thickened skin with a modified Rodnan Skin Score ≥15;
(ii) Disease duration ≤7 years; and
(iii) Anti-centromere polymerase autoantibody negative.

82. An anti-BlyS antibody for use according to any one of embodiments 67 to 77, wherein the human subject is characterised by:
(i) Presence of thickened skin with a modified Rodnan Skin Score ≥15; and
(ii) Anti-topoisomerase I (anti-Scl70) autoantibody positive.

83. An anti-BlyS antibody for use according to any one of embodiments 67 to 82, wherein the human subject has a disease duration of ≥2 years and one or more of:
(i) Reduction in forced vital capacity;
(ii) Reduction in carbon monoxide diffusing capacity;
(iii) Anti-topoisomerase I (anti-Scl70) autoantibody positive; and
(iv) Increased extent of ILD on HRCT.

84. The anti-BlyS antibody for use according to any one of embodiments 67 to 83, wherein the antibody comprises: CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6 or variants thereof.

85. The anti-BlyS antibody for use according to any one of embodiments 67 to 84, wherein the antibody comprises: CRDH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6.

86. The anti-BlyS antibody for use according to any one of embodiments 67 to 85, wherein the antibody comprises a variable heavy chain sequence of SEQ ID NO: 7, or a sequence which is at least 90% identical thereto, and a variable light chain sequence of SEQ ID NO: 8, or a sequence which is at least 90% identical thereto.

87. The anti-BlyS antibody for use according to any one of embodiments 67 to 86, wherein the antibody comprises a variable heavy chain sequence of SEQ ID NO: 7 and a variable light chain sequence of SEQ ID NO: 8.

88. The anti-BlyS antibody for use according to any one of embodiments 67 to 87, wherein the antibody is belimumab.

89. The anti-BlyS antibody for use according to any one of embodiments 67 to 88, wherein the antibody is administered subcutaneously.

90. The anti-BlyS antibody for use according to embodiment 89, wherein the antibody is administered to the human subject at a unit dose of 200 mg per week.

91. The anti-BlyS antibody for use according to any one of embodiments 67 to 90, wherein the antibody is co-administered with an additional immunosuppressive agent.

92. The anti-BlyS antibody for use according to embodiment 91, wherein the additional immunosuppressive agent is selected from the group methotrexate, mycophenolate mofetil, mycophenolate sodium, or azathioprine.

93. A method of treating Systemic sclerosis-associated interstitial lung disease using the anti-BlyS antibody according to any one of embodiments 67 to 92.

94. A method of improving the quality of life of a subject suffering from early diffuse cutaneous systemic sclerosis-associated interstitial lung disease, wherein the subject is administered a therapeutically suitable amount of an anti-BlyS antibody.

95. A BlyS antagonist for use in the treatment of autoimmune interstitial lung disease in a human subject in need thereof.

96. A BlyS antagonist for use according to embodiment 95, wherein the autoimmune interstitial lung disease is connective tissue disease-associated interstitial lung disease.

97. A BlyS antagonist for use according to embodiment 96, wherein the connective tissue disease-associated interstitial lung disease comprises systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease.

98. A BlyS antagonist for use according to embodiment 97, wherein the connective tissue disease-associated interstitial lung disease does not comprise systemic lupus erythematosus-associated interstitial lung disease.

99. A BlyS antagonist for use according to embodiment 97, wherein the systemic sclerosis-associated interstitial lung disease does not further comprise systemic lupus erythematosus or systemic lupus erythematosus-associated interstitial lung disease.

100. A BlyS antagonist for use according to any one of embodiments 97 to 99, wherein the connective tissue disease-associated interstitial lung disease consists of systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease.

101. A BlyS antagonist for use according to any one of embodiments 96 to 100, wherein the interstitial lung disease does not comprise usual interstitial pneumonia.

102. A BlyS antagonist for use according to any one of embodiments 96 to 100, wherein the interstitial lung disease comprises usual interstitial pneumonia.

103. A BlyS antagonist for use according to any one of embodiments 96 to 102, wherein the interstitial lung disease comprises nonspecific interstitial pneumonia, organising pneumonia, and/or lymphocytic interstitial pneumonia.

104. A BlyS antagonist for use according to embodiment 102, wherein the connective tissue disease-associated interstitial lung disease is rheumatoid arthritis-associated interstitial lung disease.

105. A BlyS antagonist for use according to any one of embodiments 96 to 103, wherein the connective tissue disease-associated interstitial lung disease is systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease, and wherein the interstitial lung disease comprises nonspecific interstitial pneumonia.

106. A BlyS antagonist for use according to any one of embodiments 96 to 103, wherein the connective tissue disease-associated interstitial lung disease is Sjögren's syndrome-associated interstitial lung disease, and wherein the interstitial lung disease comprises lymphocytic interstitial pneumonia.

107. A BlyS antagonist for use according to any one of embodiments 96 to 103, wherein the connective tissue disease-associated interstitial lung disease is an idiopathic inflammatory myopathy-associated interstitial lung disease, and wherein the interstitial lung disease comprises organising pneumonia.

108. A BlyS antagonist for use according to embodiment 96, wherein the connective tissue disease-associated interstitial lung disease is characterised by one or more of.
(i) the connective tissue disease does not comprise systemic lupus erythematosus; and
(ii) the interstitial lung disease comprises usual interstitial pneumonia.

109. A BlyS antagonist for use according to embodiment 108, part (i), wherein the interstitial lung disease does not comprise usual interstitial pneumonia.

110. A BlyS antagonist for use according to embodiment 108 or 109, wherein the interstitial lung disease comprises nonspecific interstitial pneumonia, organising pneumonia, and/or lymphocytic interstitial pneumonia.

111. A BlyS antagonist for use according to any one of embodiments 108 to 110, wherein the connective tissue disease comprises systemic sclerosis, rheumatoid arthritis, Sjögren's syndrome, an idiopathic inflammatory myopathy, and/or mixed connective tissue disease.

112. A BlyS antagonist for use according to embodiment 108, part (ii), wherein the connective tissue disease comprises systemic lupus erythematosus.

113. A BlyS antagonist for use according to embodiment 108, part (ii), wherein the connective tissue disease comprises rheumatoid arthritis.

114. A BlyS antagonist for use according to any one of embodiments 108 to 111, wherein the connective tissue disease comprises systemic sclerosis, rheumatoid arthritis, Sjögren's syndrome, an idiopathic inflammatory myopathy, and/or mixed connective tissue disease, and wherein the interstitial lung disease comprises nonspecific interstitial pneumonia.

115. A BlyS antagonist for use according to any one of embodiments 108 to 111, wherein the connective tissue disease comprises Sjögren's syndrome, and wherein the interstitial lung disease comprises lymphocytic interstitial pneumonia.

116. A BlyS antagonist for use according to any one of embodiments 108 to 111, wherein the connective tissue disease comprises an idiopathic inflammatory myopathy, and wherein the interstitial lung disease comprises organising pneumonia.

117. A BlyS antagonist for use according to any one of embodiments 96 to 103, 105, 107, 111, 114, or 116, wherein the idiopathic inflammatory myopathy comprises polymyositis, dermatomyositis, or anti-synthetase syndrome.

118. A BlyS antagonist for use according to any one of embodiments 96 to 117, wherein the treatment is characterised by a reduction in disease progression.

119. A BlyS antagonist for use according to embodiment 118, wherein the disease progression is connective tissue disease progression and/or interstitial lung disease progression.

120. A BlyS antagonist for use according to embodiment 119, wherein the disease progression is interstitial lung disease progression, and wherein the interstitial lung disease progression is characterised by a reduction in forced vital capacity.

121. A BlyS antagonist for use according to embodiment 120, wherein the interstitial lung disease progression is further characterised by:
(i) a reduction in forced vital capacity of ≥5% relative to baseline, optionally, a reduction in forced vital capacity of ≥10% relative to baseline; or
(ii) a reduction in forced vital capacity of ≥5% to <10% relative to baseline and a reduction in diffusing capacity of the lung for carbon monoxide of ≥15% relative to baseline.

122. A BlyS antagonist for use according to embodiment 119, wherein the disease progression is interstitial lung disease progression, and wherein the interstitial lung disease progression is characterised by two or more of the following (a) to (c) occurring within a 12 month time period:
(a) a worsening of respiratory symptoms;
(b) a physiological evidence of interstitial lung disease progression, wherein the physiological evidence of interstitial lung disease progression comprises:
   (i) an absolute reduction in forced vital capacity of ≥5% predicted; and/or
   (ii) an absolute reduction in diffusing capacity of the lung for carbon monoxide of ≥10% predicted; and/or
(c) a radiological evidence of interstitial lung disease progression.

123. A BlyS antagonist for the use according to embodiment 120, wherein the interstitial lung disease progression is characterised by a reduction in forced vital capacity of ≥10% relative to baseline.

124. A BlyS antagonist for the use according to embodiment 118, wherein the reduction in disease progression is a reduction in interstitial lung disease progression, wherein the reduction in interstitial lung disease progression is characterised by a reduction in forced vital capacity of no more than 9%, no more than 8%, no more than 7%, no more than 6%, no more than 5%, no more than 4%, no more than 3%, no more than 2%, or no more than 1% relative to baseline.

125. A BlyS antagonist for the use according to embodiment 119, wherein the disease progression is interstitial lung disease progression, and wherein the interstitial lung disease progression is characterised by a reduction in diffusing capacity of the lung for carbon monoxide.

126. A BlyS antagonist for the use according to embodiment 125, wherein the interstitial lung disease progression is characterised by a reduction in diffusing capacity of the lung for carbon monoxide of ≥15% relative to baseline.

127. A BlyS antagonist for the use according to embodiment 118, wherein the reduction in disease progression is a reduction in interstitial lung disease progression, wherein the reduction in interstitial disease progression is characterised by a reduction in diffusing capacity of the lung for carbon monoxide of no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, no more than 1%, or 0% relative to baseline.

128. A BlyS antagonist for use according to any one of embodiments 119 to 127, wherein the interstitial lung disease progression further comprises progressive pulmonary fibrosis.

129. A BlyS antagonist for use according to any one of embodiments 96 to 128, wherein the treatment is characterised by:
(a) an improvement in one or more measures relative to baseline in the human subject, wherein the one or more measures are selected from:
   (i) forced vital capacity;
   (ii) diffusing capacity of the lung for carbon monoxide;
   (iii) connective tissue disease-associated interstitial lung disease symptom severity; and/or
   (iv) quality of life, and/or
(b) a reduction in one or more measures relative to baseline in the human subject, wherein the one or more measures are selected from:
   (iv) extent of interstitial lung disease; and/or
   (v) corticosteroid use.

130. A BlyS antagonist for use according to embodiment 129, wherein the improvement in connective tissue disease-associated interstitial lung disease symptom severity comprises a reduction in fatigue, a reduction in dyspnea and/or a reduction in cough relative to baseline.

131. A BlyS antagonist for use according to embodiment 130, wherein the reduction in fatigue is measured by an increase in a Functional Assessment of Chronic Illness Therapy (FACIT)-Fatigue score or by a decrease a Living with Idiopathic Pulmonary Fibrosis (L-PF) Symptoms score within the fatigue domain.

132. A BlyS antagonist for use according to embodiment 130 or 131, wherein the reduction in dyspnea is measured by a decrease in a Living with Idiopathic Pulmonary Fibrosis (L-PF) Symptoms score within the dyspnea domain or by an increase in a Transition Dyspnea Index score.

133. A BlyS antagonist for use according to any one of embodiments 130 to 132, wherein the reduction in cough is measured by a decrease in a Living with Idiopathic Pulmonary Fibrosis (L-PF) Symptoms score within the cough domain.

134. A BlyS antagonist for use according to any one of embodiments 96 to 133, wherein the treatment is characterised by an increase in progression free survival.

135. A BlyS antagonist for use according to any one of embodiments 96 to 134, wherein the human subject is characterised by an extent of interstitial lung disease that is ≥10% of the whole lung or by an extent of fibrosis that is ≥10% of the whole lung.

136. A BlyS antagonist for use according to embodiment 135, wherein the extent of interstitial lung disease or the extent of fibrosis is measured prior to a first administration of the BlyS antagonist, optionally, wherein the extent of interstitial lung disease is measured by high-resolution computed tomography.

137. A BlyS antagonist for use according to any one of embodiments 95 to 136, wherein the BlyS antagonist is an anti-BlyS antibody.

138. An anti-BlyS antibody for use according to embodiment 137, wherein the antibody comprises: CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6 or variants thereof.

139. An anti-BlyS antibody for use according to embodiment 137 or 138, wherein the antibody comprises: CRDH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6.

140. An anti-BlyS antibody for use according to any one of embodiments 137 to 139, wherein the antibody comprises a variable heavy chain sequence of SEQ ID NO: 7, or a sequence which is at least 90%, at least 91%, at least 92%, at least 93%, at least 94%, at least 95%, at least 96%, at least 97%, at least 98%, or at least 99% identical thereto, and a variable light chain sequence of SEQ ID NO: 8, or a sequence which is at least 90%, at least 91%, at least 92%, at least 93%, at least 94%, at least 95%, at least 96%, at least 97%, at least 98%, or at least 99% identical thereto.

141. An anti-BlyS antibody for use according to any one of embodiments 137 to 140, wherein the anti-BlyS antibody comprises a variable heavy chain sequence of SEQ ID NO: 7 and a variable light chain sequence of SEQ ID NO: 8.

142. An anti-BlyS antibody for use according to any one of embodiments 137 to 141, wherein the anti-BlyS antibody comprises a variable heavy chain sequence of SEQ ID NO: 7 and a variable light chain sequence of amino acids 1 to 108 of SEQ ID NO: 8.

143. An anti-BlyS antibody for use according to any one of embodiments 137 to 142, wherein the anti-BlyS antibody comprises a heavy chain sequence of SEQ ID NO: 9 and a light chain sequence of SEQ ID NO: 10.

144. An anti-BlyS antibody for use according to any one of embodiments 137 to 143, wherein the anti-BlyS antibody is belimumab.

145. An anti-BlyS antibody for use according to any one of embodiments 137 to 144, wherein the anti-BlyS antibody is administered subcutaneously.

146. An anti-BlyS antibody for use according to embodiment 145, wherein the anti-BlyS antibody is administered to the human subject at a unit dose of 200 mg per week.

147. An anti-BlyS antibody for use according to any one of embodiments 137 to 144, wherein the anti-BlyS antibody is administered intravenously.

148. An anti-BlyS antibody for use according to embodiment 147, wherein the anti-BlyS antibody is administered to the human subject at a unit dose of 10 mg/kg.

149. An anti-BlyS antibody for use according to any one of embodiments 137 to 148, wherein the anti-BlyS antibody is co-administered with an additional immunosuppressive agent.

150. An anti-BlyS antibody for use according to embodiment 149, wherein the additional immunosuppressive agent is selected from methotrexate, mycophenolate mofetil, mycophenolate sodium, azathioprine, a corticosteroid, a calcineurin inhibitor, a pyrimidine synthesis inhibitor, and/or an anti-malarial agent.

151. An anti-BlyS antibody for use according to embodiment 150, wherein the calcineurin inhibitor is tacrolimus or cyclosporin.

152. An anti-BlyS antibody for use according to embodiment 150, wherein the pyrimidine synthesis inhibitor is leflunomide.

153. An anti-BlyS antibody for use according to embodiment 150, wherein the anti-malarial agent is hydroxychloroquine.

154. An anti-BlyS antibody for use according to any one of embodiments 137 to 153, wherein the anti-BlyS antibody is co-administered with an additional anti-fibrotic agent.

155. An anti-BlyS antibody for use according to embodiment 154, wherein the additional anti-fibrotic agent is nintedanib.

156. A pharmaceutical composition comprising the BlyS antagonist according to any one of embodiments 96 to 136 or the anti-BlyS antibody according to any one of embodiments 137 to 155 and a pharmaceutically acceptable excipient, for use in the treatment of connective tissue disease-associated interstitial lung disease.

157. Use of the BlyS antagonist according to any one of embodiments 96 to 136, or the anti-BlyS antibody according to any one of embodiments 137 to 155, in the manufacture of a medicament for use in the treatment of connective tissue disease-associated interstitial lung disease.

158. A method for the treatment of connective tissue disease-associated interstitial lung disease in a human subject in need thereof comprising administering to the human subject a therapeutically effective amount of the BlyS antagonist according to any one of embodiments 96 to 136, the anti-BlyS antibody according to any one of embodiments 137 to 155, or the pharmaceutical composition according to embodiment 156.

159. A method of improving the quality of life of a human subject suffering from connective tissue disease-associated interstitial lung disease, wherein the human subject is administered a therapeutically suitable amount of the BlyS antagonist according to any one of embodiments 96 to 136, the anti-BlyS antibody according to any one of embodiments 137 to 155, or the pharmaceutical composition according to embodiment 156.

### EXAMPLES

Despite recent approvals, there remains a significant unmet need for targeted therapies that offer SSc-ILD subjects benefit beyond lung function. Furthermore, there remains a significant unmet need for therapies for CTD-ILD.

We hypothesise that elevated BlyS in early dcSSc subjects will promote survival and generation of autoreactive B cells which accumulate in the diseased tissues (skin and lung) in SSc, and that these infiltrating autoreactive B cells contribute to pathogenesis by multiple effector mechanisms. Mechanisms may include B cell-cytokine release, e.g., IL-6, activation of fibroblasts by B cell-fibroblast direct contact, cognate activation of autoreactive T cells which produce pro-fibrotic T-cell cytokines, e.g., IL13, and production of autoantibodies which form immune complexes promoting tissue damage (activation of innate immune cells via FcRs and ADCC).

Although it has been previously described that circulating BlyS protein is increased in SSc subject serum and that BlyS gene expression is enhanced in early dcSSc skin versus healthy control samples (Matsushita, et al., (2006) Arthritis Rheum. 2006; 54(1):192-201; Minh, et al., (2019) Open Access Maced J Med Sci. 2019 Jan. 28; 7(2):264-268; Fawzy, et al., (2011) The Egyptian Rheumatologist 2011; 33(1):45-51), it was not yet understood how the presence of BlyS may be contributing to the disease pathology.

It is hypothesised that BlyS is produced locally in tissue, probably from infiltrating immune cells and potentially fibroblasts, helps maintain the survival, expansion, and activation of B cells within SSc tissues. These cells contribute to pathogenesis early in disease and in the development of fibrotic tissues. Targeting BlyS will reduce the survival and activation of B cells in tissue and block the disease progression.

With regard to CTD more generally, without wishing to be bound by theory, it is hypothesised that in CTD, immune mediated microvascular injury may lead to damage to the lung epithelial cells which then triggers inflammation resulting in activation and infiltration of B and T cells leading to cytokine release systemically and within the lung. B-cells are reported to play an important role in the pathogenesis of ILD. Persistent inflammation may lead to tissue injury and eventual fibrosis that results in thickening of the alveolar wall with consequent impairment of gas exchange. Without wishing to be bound by any particular theory, it is further hypothesised that treatment with a BlyS antagonist may reduce the number of circulating autoreactive B cells and/or promote a shift towards a more regulatory B cell profile. This may lead to reduced inflammation and fibrosis across multiple organ systems, including the lung. Therefore, by targeting BlyS, the survival and activation of B cells in tissue may be reduced, leading to reduced disease progression.

### Example 1—Evaluation of B Cell Gene Expression in SSc Skin Samples

The expression of B-cell related genes in SSc skin samples was explored using Gene Set Variance Analyses (GSVA).

A specific novel set of gene lists representing different B cell subsets (Activated B/Atypical B cells, Generic Activated B cells, Generic B cells, Germinal Centre B cells, Memory B cells, and Plasmablasts/Plasma cells) were designed based on genes known to be expressed in these functional subsets. A random gene set of a similar size was used as a control reference set.

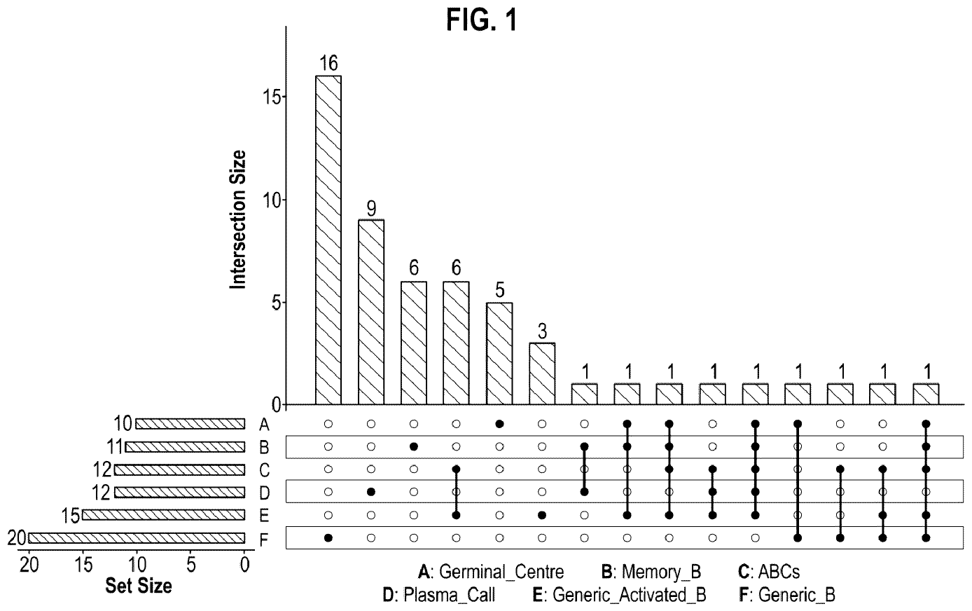

Of particular interest were atypical B cells (also called age-related B cells) (FIG. 1) which have been previously associated with autoimmunity (Mouat, et al., (2022) Cell. Mol. Life Sci. 2022; 79:402). The gene lists were relatively short, and some genes overlapped (for example, CD19 is present in all B cells), thus whilst the results for B cell gene-set enrichment can be a guide to presence of specific B cell types, additional experimental validation is planned.

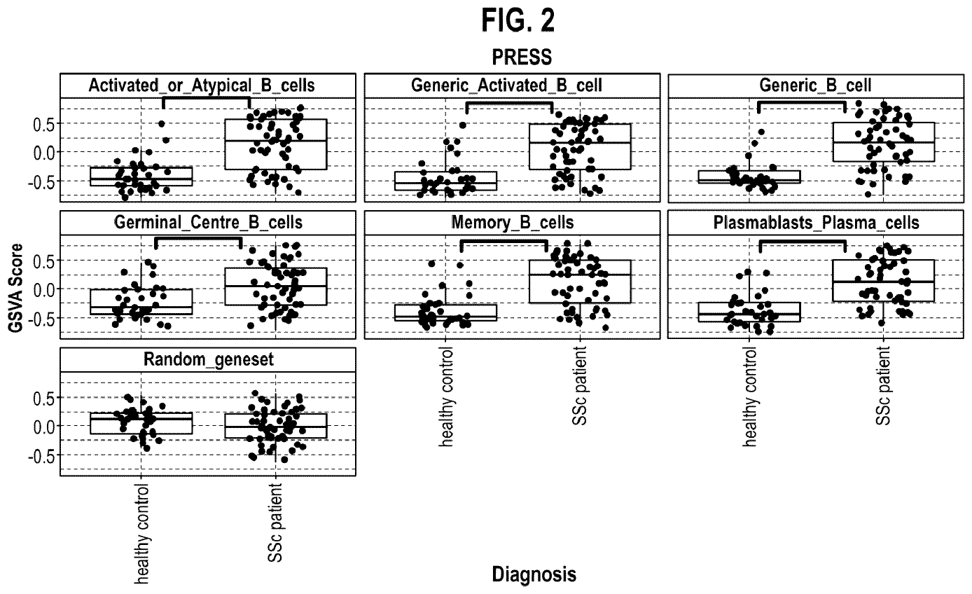

Using GSVA on our gene signature we observed a significant enhanced enrichment of B cell related genes including those associated with activated B cells in early dcSSc skin samples versus healthy control skin samples in the Prospective Registry for Early Systemic Sclerosis (PRESS) cohort These results demonstrate B cells are present in SSc skin and are supportive of the hypothesis for activated B cells playing a role in disease. (FIG. 2).

### Example 2—B Cell Gene Sets and Disease Measures in dcSSc Skin

We next examined the relationship of the B cell expression to a previously derived SSc skin disease signature and the Modified Rodnan Skin Scores (mRSS) disease score to show the relationship in diffuse cutaneous systemic sclerosis subjects in the PRESS cohort.

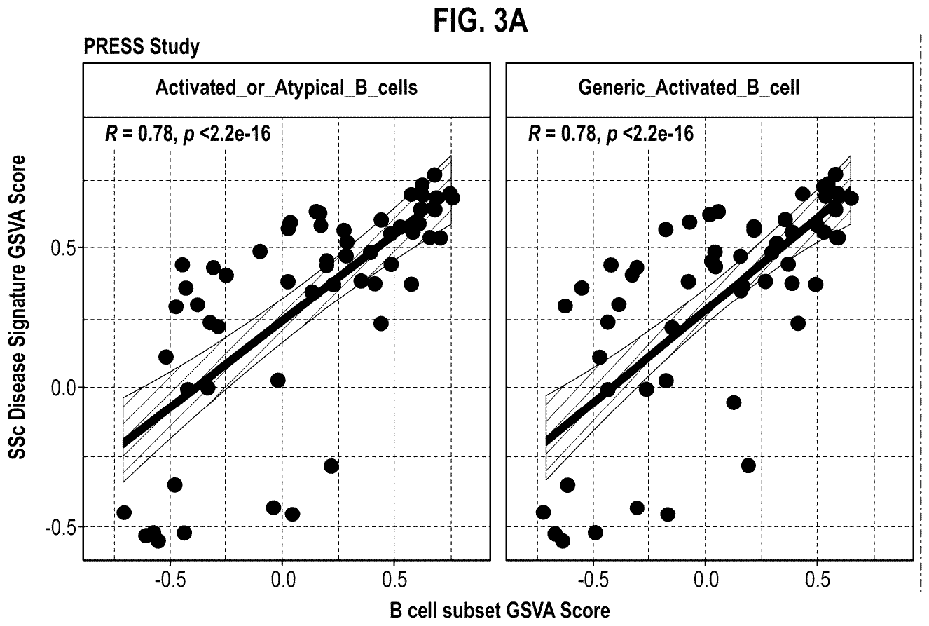

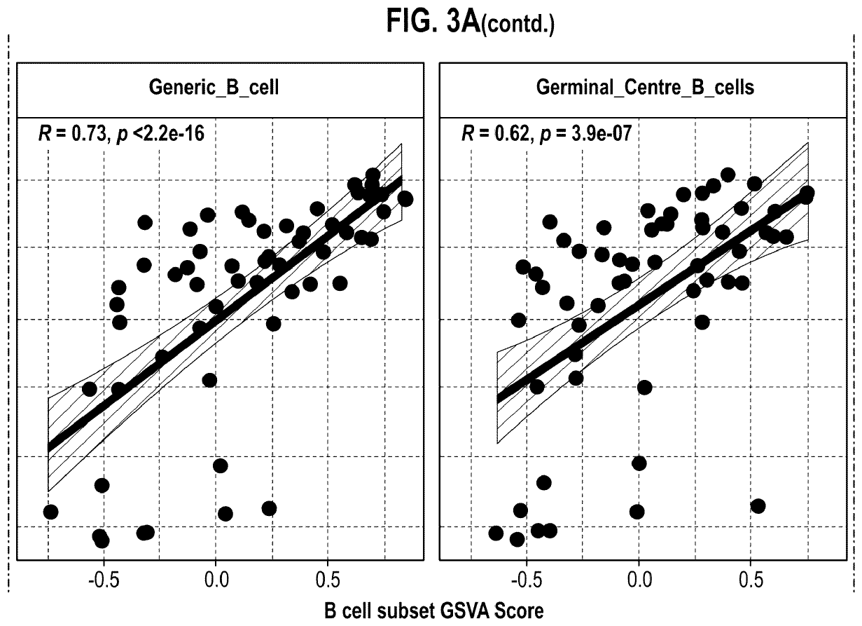

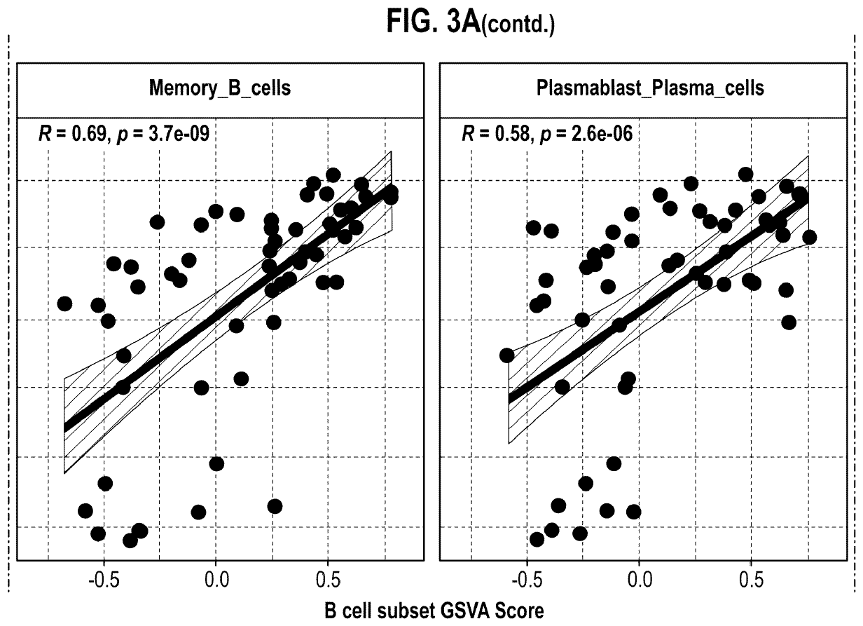

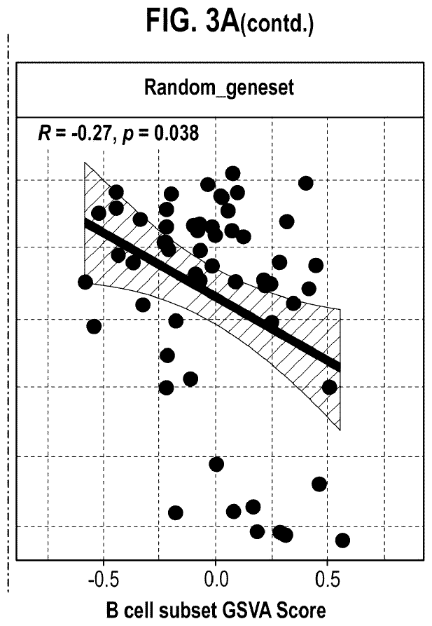

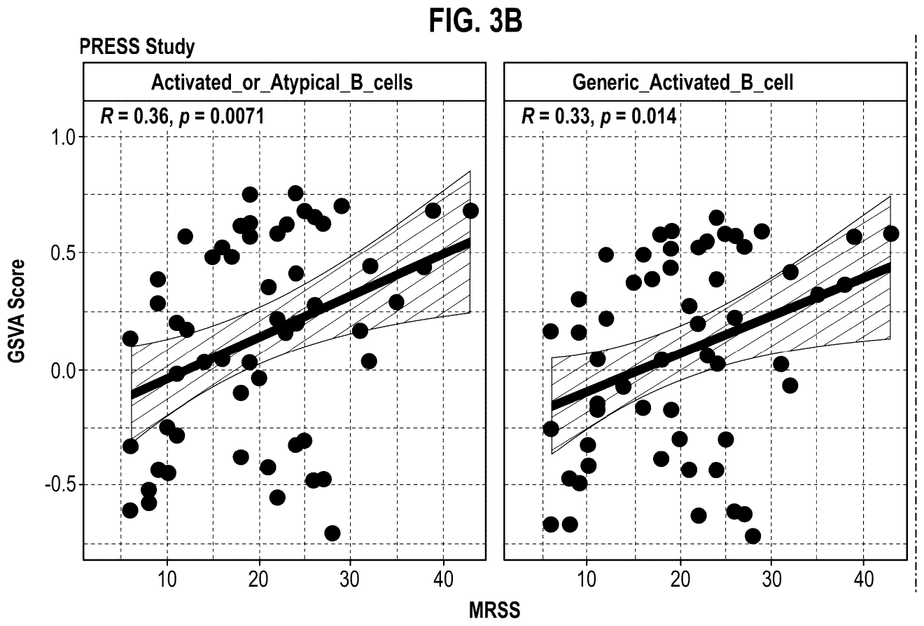

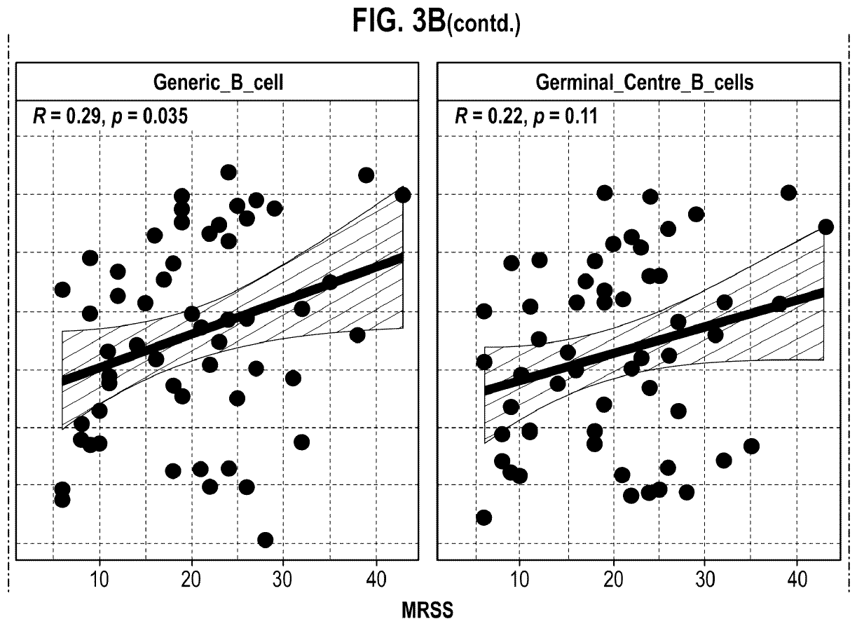

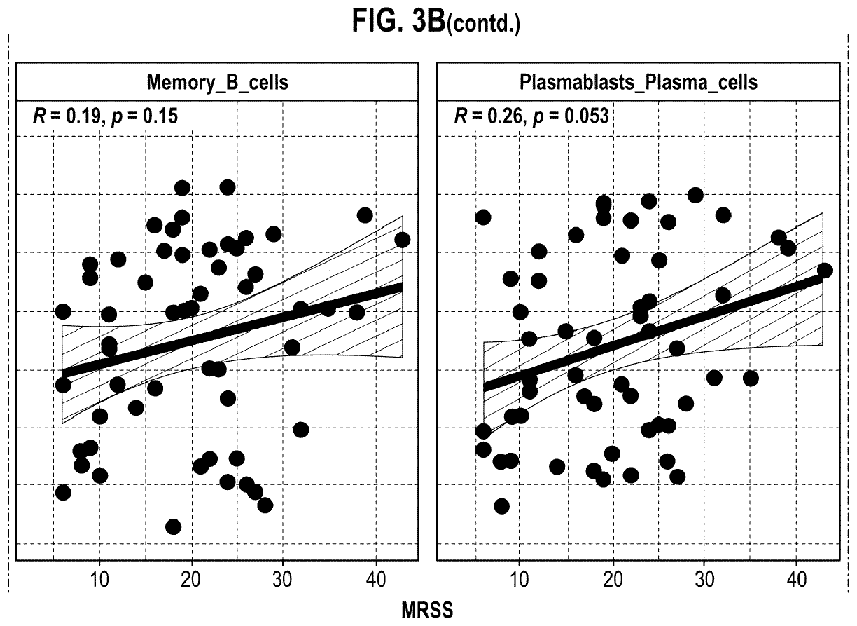

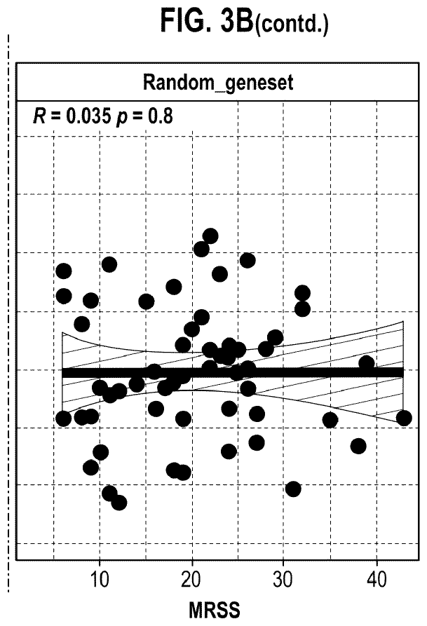

There was a moderate to strong positive correlation (R ≥0.58 and p-value <0.05) between B cell enrichment scores for all subsets in early dcSSc skin with the disease signature (FIG. 3A) and a weak positive correlation (R ≥0.29 and p-value <0.05) between the Activated or Atypical B cell, Generic Activated B cell, and Generic B cell signatures with the mRSS (FIG. 3B). The random gene set did not show a correlation with the mRSS and showed a weak negative correlation with the disease signature. These results further support a role for B cells in the disease process in SSc tissues.

### Example 3—Belimumab Treatment Reduced Expression of Genes Associated with Activated B Cells in SSc Skin

A previous clinical study explored the effect of belimumab treatment in combination with Standard of Care in dcSSc subjects, and global gene expression data were derived from the pre- and post-treatment skin biopsy samples from this study (Gordon et al., (2018) Arthritis Rheumatol. 2018; 70(2):308-16. Doi: 10.1002/art.40358).

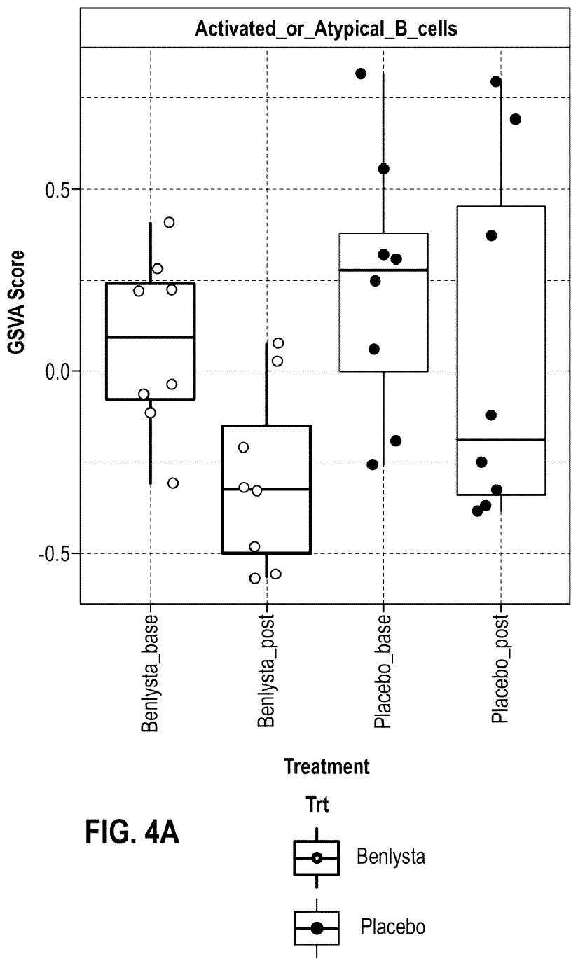

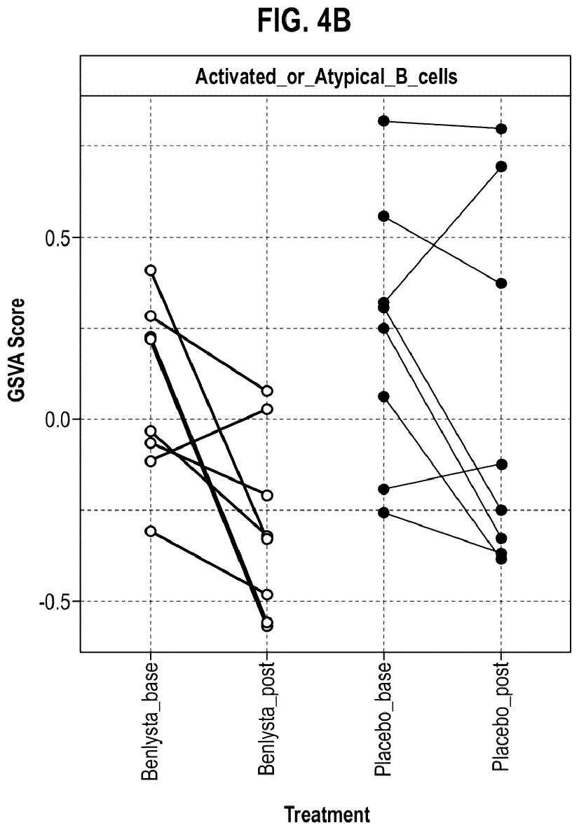

Using this data, we further evaluated changes in enrichment of the B cell gene sets across the treatment groups using GSVA and observed a reduction in the Activated/Atypical cell gene set enrichment scores following belimumab treatment which was not observed in the PBO treated group. (Pairwise Wilcox-test for belimumab baseline versus post-baseline comparison p-value: 0.044; Placebo baseline versus post-baseline p-value: 0.459). This showed that belimumab has potential to reduce B cell activation in the skin of subjects. (See FIGS. 4A and 4B).

### Example 4—B Cell Gene Sets and Disease Measures in Lung

As a result of the data seen above in skin, the inventors then looked into whether the same gene expression and B cell subsets could be seen in the lung tissue of Systemic sclerosis subjects using published lung single cell RNA seq data sets from 3 different studies.

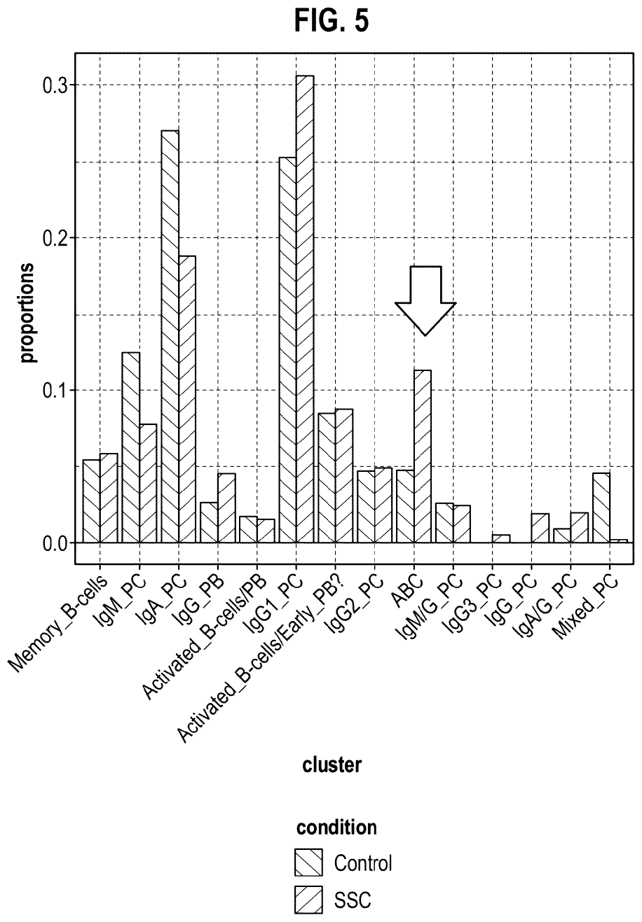

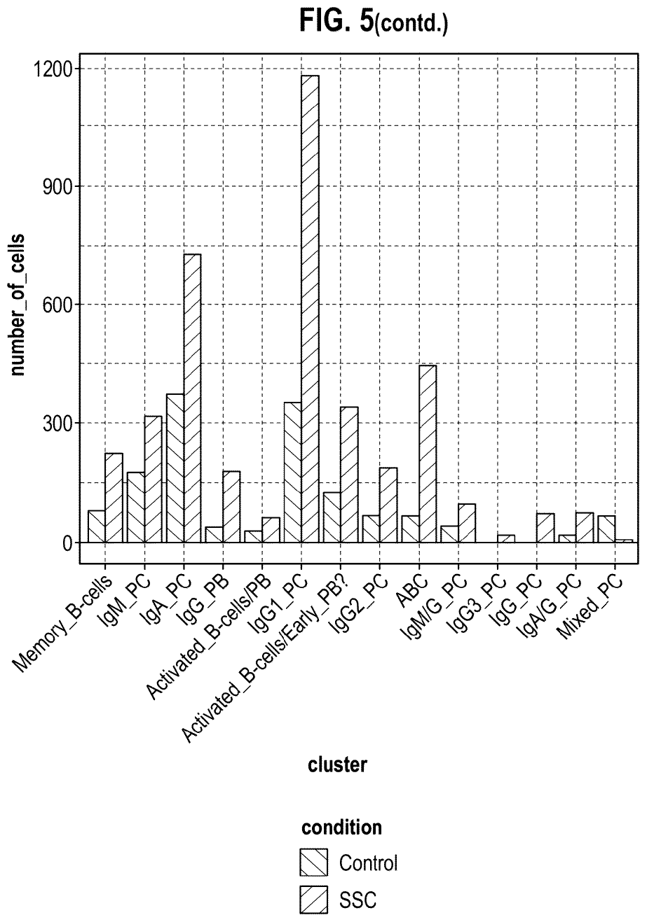

Although formal assessment of differential expression in healthy samples is lacking, this was believed to be due to the limited small sample sizes and technical variations in samples and gave us further confidence in the hypothesis that B cells are involved in SSc lung disease. (FIG. 5).

Further work to confirm this hypothesis will be carried out.

### Example 5—Planned Clinical Study (SSc-ILD)

As a result of the increased understanding that BlyS can impact subjects affected with Systemic sclerosis, a Phase III clinical trial is planned according to the following protocol. Title: A Phase 2/3, randomized, double-blind, placebo-controlled, parallel-group study to evaluate the efficacy and safety of belimumab administered subcutaneously in adults with systemic sclerosis associated interstitial lung disease (SSc-ILD).

This study investigates the efficacy and safety of belimumab compared to placebo, in addition to standard therapy, for the treatment of participants with systemic sclerosis associated interstitial lung disease (SSc-ILD). The study will evaluate the effect of belimumab treatment on lung function as well as on extra-pulmonary disease manifestations, including skin thickening and general symptoms, such as fatigue, that impact quality of life (QoL).

Participants are randomly assigned (1:1) to 1 of 2 treatment arms:
Belimumab 200 mg, subcutaneous administration (SC), in addition to standard therapy; or
Placebo SC in addition to standard therapy.

Randomization (Day 1) and the first dose of study treatment are completed within 6 weeks after initiation of screening procedures. If participants are receiving stable background immunosuppressive therapy at entry this should remain stable throughout the trial.

Belimumab or placebo are administered SC on Day 1 and then weekly (i.e., every 7 days±3 days) until Week 51.

The maximum duration of participation is expected to be no greater than 66 weeks for any participant in the study, including:
Screening—up to 6 weeks.
Double-blind treatment phase—up to 52 weeks.
Safety follow-up visit 8 weeks after discontinuation of study intervention.

Main Objective: To evaluate the efficacy of belimumab compared to placebo, in addition to standard therapy, on reducing the decline in lung volume in participants with dcSSc-ILD, as measured by change from baseline in FVC.

### Secondary Objectives:

1. To evaluate the efficacy of belimumab compared to placebo, in addition to standard therapy, on reducing the extent of skin thickening in participants with dcSSc-ILD at Week 52.
2. To evaluate the efficacy of belimumab compared to placebo, in addition to standard therapy, on reducing fatigue in participants with dcSSc-ILD.
3. To evaluate the efficacy of belimumab compared to placebo, in addition to standard therapy, on SSc progression or mortality in participants with dcSSc-ILD.
4. To evaluate the efficacy of belimumab compared to placebo, in addition to standard therapy, on reducing the decline in % predicted lung volume in participants with dcSSc-ILD.
5. To evaluate the efficacy of belimumab compared to placebo, in addition to standard therapy, on relative decline in lung volume in participants with dcSSc-ILD.
6. To evaluate the efficacy of belimumab compared to placebo, in addition to standard therapy, on reducing the extent of skin thickening in participants with dcSSc-ILD at Week 26.
7. To evaluate the efficacy of belimumab compared to placebo, in addition to standard therapy, on relative worsening of skin thickening in participants with dcSSc-ILD.
8. To evaluate the efficacy of belimumab compared to placebo, in addition to standard therapy, on maintenance of pulmonary structure as assessed by quantitative HRCT in participants with dcSSc-ILD.
9. To evaluate the efficacy of belimumab compared to placebo, in addition to standard therapy, on reducing the decline in pulmonary gas transfer in participants with dcSSc-ILD.
10. To evaluate the effect of belimumab compared to placebo, in addition to standard therapy, on dcSSc-ILD related symptoms and health-related quality of life by patient and physician reported outcomes in participants with dcSSc-ILD.
11. To evaluate the safety and tolerability of belimumab compared to placebo, in addition to standard therapy, in treatment of participants with dcSSc-ILD, as measured by occurrence of adverse events.

### Primary and Secondary Outcome Measures

| | |
|---|---|
| Outcome measure type | Primary |
| Outcome measure title | Absolute change from baseline in Forced |
| | Vital Capacity (FVC) millilitre (mL) at |
| | Week 52 |
| Outcome measure type | Secondary |
| Outcome measure title | Absolute change from baseline in modified |
| | Rodnan Skin Score (mRSS) at Week 52 |
| Outcome measure description | The modified Rodnan Skin Score (mRSS) is |
| | an evaluation of the patient's skin thickness |
| | rated by clinical palpation using a 0 to 3 |
| | scale. The scale differentiates between |
| | 0 = normal skin, 1 = mild thickness, |
| | 2 = moderate thickness, and 3 = severe |
| | thickness with inability to pinch the skin |
| | into a fold. The assessment is made across |
| | 17 pre-defined areas of the body and |
| | therefore the total score can range from |
| | 0 to 51. Higher scores indicate worse skin |
| | thickening. |
| Outcome measure type | Secondary |
| Outcome measure title | Absolute change from baseline in |
| | Functional Assessment of Chronic Illness |
| | Therapy (FACIT)-Fatigue score at Week 52 |
| Outcome measure description | FACIT-fatigue is a validated patient- |
| | reported measure developed originally to |
| | assess fatigue in individuals with cancer and |
| | has subsequently been used and validated in |
| | numerous chronic conditions, including |
| | SSc. FACIT-Fatigue scores range from 0-52 |
| | (higher scores indicate less fatigue). |
| Outcome measure type | Secondary |
| Outcome measure title | Time to Systemic sclerosis (SSc) |
| | progression or death |
| Outcome measure description | SSc progression or death is defined as the |
| | time when major organ-based complications |
| | develop, or the participant dies. |
| Time frame | From the date of assignment until the date |
| | of first documented progression or date of |
| | death from any cause, whichever comes |
| | first, assessed up to 52 Weeks |
| Outcome measure type | Secondary |
| Outcome measure title | Absolute change from baseline in FVC |
| | percentage (%) predicted at Week 52 |
| Outcome measure type | Secondary |
| Outcome measure title | Relative decline from baseline in FVC (mL) |
| | greater than or equal to (≥)5% at Week 52 |
| Outcome measure type | Secondary |
| Outcome measure title | Relative decline from baseline in FVC (mL) |
| | ≥10% at Week 52 |
| Outcome measue type | Secondary |
| Outcome measure title | Absolute change from baseline in mRSS at |
| | Week 26 |
| Outcome measure description | The modified Rodnan Skin Score (mRSS) is |
| | an evaluation of the patient's skin thickness |
| | rated by clinical palpation using a 0 to 3 |
| | scale. The scale differentiates between |
| | 0 = normal skin, 1 = mild thickness, |
| | 2 = moderate thickness, and 3 = severe |
| | thickness with inability to pinch the skin |
| | into a fold. The assessment is made across |
| | 17 pre-defined areas of the body and |
| | therefore the total score can range from |
| | 0 to 51. Higher scores indicate worse skin |
| | thickening. |
| Outcome measure type | Secondary |
| Outcome measure title | Proportion of participants achieving ≥20% |
| | increase in mRSS at Week 26 & 52 |
| Outcome measure type | Secondary |
| Outcome measure title | Absolute change from baseline in |
| | Quantitative interstitial lung disease-whole |
| | lung (QILD-WL) at Week 52 |
| Outcome measure type | Secondary |
| Outcome measure title | Absolute change from baseline in |
| | Quantitative lung fibrosis-whole lung |
| | (QLF-WL) at Week 52 |
| Outcome measure type | Secondary |
| Outcome measure title | Proportion of participants achieving ≥2% |
| | increase in QILD at Week 52 |
| Outcome measure type | Secondary |
| Outcome measure title | Absolute change from baseline in Dlco % |
| | predicted at Week 52 |
| Outcome measure type | Secondary |
| Outcome measure title | Relative decline from baseline in Dlco % |
| | predicted ≥15% at Week 52 |
| Outcome measure type | Secondary |
| Outcome measure title | Absolute change from baseline in Cough |
| | Numeric Rating Scale (NRS) at Week 52 |
| Outcome measure description | The cough NRS enables the participant to |
| | rate their cough on a defined scale from 0 to |
| | 10, where higher score indicating worse |
| | cough symptoms |
| Time frame | Baseline and Week 52 |
| Outcome measure type | Secondary |
| Outcome measure title | Absolute change from baseline in |
| | Scleroderma Skin Patient-Reported |
| | Outcome (SSPRO) at Week 52 |
| Outcome measure description | SSPRO a patient-reported outcome (PRO) |
| | instrument developed to assess the skin- |
| | related quality of life (QoL) in participants |
| | with SSc. SSPRO has 18 items that assess |
| | four SSc skin-related HRQOL domain |
| | (emotional effects, physical effects, |
| | physical limitations, and social effects). |
| | The higher score indicates worse impact |
| | on QoL. |
| Time frame | Baseline and Week 52 |
| Outcome measure type | Secondary |
| Outcome measure title | Absolute change from baseline in Health |
| | Assessment Questionnaire Disability Index |
| | (HAQ-DI) at Week 52 |
| Outcome measure description | The HAQ-DI is a 26-question instrument |
| | assessing the degree of difficulty in |
| | accomplishing tasks in eight functional |
| | areas: dressing and grooming, arising, |
| | eating, walking, hygiene, reach, grip, and |
| | common daily activities. Each question is |
| | scored 0-3 (where 0 = without difficulty & |
| | 3 = unable to do). Higher scores reflect worse |
| | disability |
| Time frame | Baseline and Week 52 |
| Outcome measure type | Secondary |
| Outcome measure title | Absolute change from baseline in Short |
| | Form-36 Health Survey Questionnaire |
| | (SF-36) at Week 52 |
| Outcome measure description | The SF-36 yields an 8-scale profile of |
| | functional health and well-being scores as |
| | well as physical and mental component |
| | health summary scores. The SF-36 consists |
| | of eight scaled scores, which are the |
| | weighted sums of the questions in their |
| | section. Each scale is directly transformed |
| | into a 0-100 scale on the assumption that |
| | each question carries equal weight. The |
| | lower the score the more disability. |
| Time frame | Baseline and Week 52 |
| Outcome measure type | Secondary |
| Outcome measure title | Absolute change from baseline in Patient |
| | Global Assessment of SSc Disease Activity |
| | (PtGA) at Week 52. |
| Outcome measure description | PtGA is a patient reported outcome scale |
| | designed to capture the participants overall |
| | assessment of their disease. The participants |
| | are asked to score their disease on a scale |
| | from 0 to 10 where higher score indicates |
| | higher severity |
| Time frame | Baseline and Week 52 |
| Outcome measure type | Secondary |
| Outcome measure title | Absolute change from baseline in Physician |
| | global assessment (PhGA) at Week 52 |
| Outcome measure description | The PhGA is a score which enables the |
| | treating physician to rate the participants |
| | disease on a scale from 0 to 10, where |
| | higher score indicates greater severity. |
| Time frame | Baseline and Week 52 |
| Outcome measure type | Secondary |
| Outcome measure title | Absolute change from baseline in Transition |
| | Dyspnea Index (TDI) at Week 52 |
| Outcome measure description | TDI assess dyspnea severity over 3 |
| | components: functional impairment, |
| | magnitude of task and magnitude of effort. |
| | Each component has 7 grades, ranging |
| | from −3 (major deterioration) to +3 (major |
| | improvement), which are summed to |
| | calculate a score, ranging between −9 and +9. |
| | The lower the score the more severely |
| | the participant is affected by dyspnea. |
| Time frame | Baseline and Week 52 |
| Outcome measure type | Secondary |
| Outcome measure title | Number of participants with Adverse |
| | Events (Aes), Adverse Events of special |
| | interest (AESIs) and Serious Aes (SAEs) up |
| | to Week 52 |
| Inclusion criteria | 1. Documented diagnosis of SSc as defined by the American |
| | College of Rheumatology/European League Against |
| | Rheumatism 2013 SSc classification criteria. |
| | 2. Diffuse cutaneous disease, defined as presence of thickened |
| | skin with mRSS greater than (>)0 over at least one skin area |
| | proximal to elbows and/or knees in addition to distal areas |
| | involvement on Day 1. |
| | 3. Evidence of interstitial lung disease. |
| | 4. Evidence for active or progressive disease |
| | 5. Participant has an area of uninvolved or mildly thickened |
| | skin that, in the opinion of the investigator, would allow SC |
| | injection at the abdomen or the front, middle region of the thigh. |
| | 6. Participant is capable and willing to self-administer the |
| | study medication or has a caregiver who is capable and |
| | willing to administer the study medication throughout the |
| | study. |
| | 7. A female participant is eligible to participate if she is not |
| | pregnant or breastfeeding, and one of the following |
| | conditions applies: |
| | Is a Woman of Non-Childbearing Potential |
| | (WONCBP) |
| | OR |
| | Is a Woman of Childbearing Potential (WOCBP) and |
| | using a contraceptive method that is highly effective. |
| | 8. Capable of giving signed informed consent. |
| Exclusion criteria | 1. Systemic sclerosis-like illness, including but not limited |
| | to localized scleroderma (morphoea), eosinophilic fasciitis, |
| | sclerodermoid graft-versus-host disease, fibro mucinous |
| | conditions (scleroedema, scleromyxoedema), scleroderma- |
| | like conditions that are associated with environmental |
| | chemical and drug exposure (e.g., toxic rapeseed oil, vinyl |
| | chloride, bleomycin, gadolinium-based contrast agents |
| | [nephrogenic systemic fibrosis], or due to metabolic |
| | disease). |
| | 2. Pulmonary arterial hypertension, as determined by the |
| | investigator at, or prior to first day of dosing (Day 1). |
| | 3. SSc renal crisis within 6 months prior to the first day of |
| | dosing (Day 1). |
| | 4. History or presence of cardiovascular, respiratory, hepatic, |
| | renal, gastrointestinal, endocrine, hematologic, or |
| | neurological disorders capable of significantly altering the |
| | absorption, metabolism, or elimination of drugs; |
| | constituting a risk when taking the study intervention or |
| | interfering with the interpretation of data. |
| | 5. Obstructive pulmonary disease (pre-bronchodilator |
| | FEV1/FVC lesser than [<]0.7). |

### Example 6—Planned Clinical Study (CTD-ILD)

A Phase 3 clinical trial is planned according to the following protocol.

Title: A Phase 3, randomized, double-blind, placebo-controlled, parallel group study to evaluate the efficacy and safety of belimumab administered subcutaneously in adults with interstitial lung disease (ILD) associated with connective tissue disease (CTD).

Participants are randomly assigned (1:1) to 1 of 2 treatment arms:
Treatment Arm 1: belimumab 200 mg SC (subcutaneous administration) weekly via pre-filled syringe in addition to standard therapy for 52 weeks; or
Treatment Arm 2: matching placebo SC weekly via pre-filled syringe in addition to standard therapy for 52 weeks.

Belimumab or placebo will be administered SC on Day 1 and then weekly (i.e., every 7 days±3 days) through to Week 51 (52 doses). The maximum duration of participation is expected to be no greater than 66 weeks for any participant in the study, including:
Screening—up to 6 weeks.
Double-blind treatment phase—up to 52 weeks.
Safety follow-up visit 8 weeks after discontinuation of study intervention.

The primary study objective is to evaluate the efficacy of belimumab compared to placebo, in addition to standard therapy, on reducing the decline in lung volume in participants with CTD-ILD, as measured by change from baseline in FVC (mL) at Week 52.

### Secondary Objectives:

1. To evaluate the efficacy of belimumab compared to placebo, in addition to standard therapy, on reducing the decline in % predicted lung volume in participants with CTD-ILD.
2. To evaluate the efficacy of belimumab compared to placebo, in addition to standard therapy, on slowing ILD progression in participants with CTD-ILD.
3. To evaluate the effect of belimumab compared to placebo, in addition to standard therapy, on fatigue in participants with CTD-ILD.
4. To evaluate the effect of belimumab compared to placebo, in addition to standard therapy, on reducing symptom severity in participants with CTD-ILD.
5. To evaluate the efficacy of belimumab compared to placebo, in addition to standard therapy, on maintenance of pulmonary structure as assessed by quantitative HRCT in participants with CTD-ILD.
6. To evaluate the efficacy of belimumab compared to placebo, in addition to standard therapy, on relative decline in lung volume in participants with CTD-ILD.
7. To evaluate the ability of belimumab compared to placebo, in addition to standard therapy, to support reduction of steroid use in participants with CTD-ILD.
8. To evaluate the efficacy of belimumab compared to placebo, in addition to standard therapy, on improving progression free survival in participants with CTD-ILD.
9. To evaluate the effect of belimumab compared to placebo, in addition to standard therapy, on improving CTD-ILD related symptoms and health-related quality of life in participants with CTD-ILD.
10. To evaluate the efficacy of belimumab compared to placebo in addition to standard therapy, on reducing the decline in pulmonary gas transfer in participants with CTD-ILD.
11. To evaluate the safety and tolerability of belimumab compared to placebo, in addition to standard therapy, in treatment of participants with CTD-ILD.

### Primary and Secondary Outcome Measures:

Outcome Measure Type: Primary

| | |
|---|---|
| Outcome Measure Title | Absolute change from baseline in Forced Vital Capacity (FVC) |
| | millilitre (mL) at Week 52. |

Outcome Measure Type: Secondary

| | |
|---|---|
| Outcome Measure Title | Absolute change from baseline in FVC percentage (%) predicted |
| | at Week 52. |
| Outcome Measure Title | Time to ILD progression or death. |
| | Outcome measure description: |
| | 1. ILD progression, defined as either: |
| | Relative decline in FVC ≥10%, OR |
| | Relative decline in FVC ≥5% to <10% AND relative |
| | decline in DLco ≥15%. |
| | 2. All-cause death (regardless of association with CTD). |
| Outcome Measure Title | Absolute change from baseline in Functional Assessment of |
| | Chronic Illness Therapy-(FACIT)-Fatigue score at Week 52 |
| | Outcome measure description: |
| | FACIT-Fatigue is a validated patient-reported measure developed |
| | originally to assess fatigue in individuals with cancer and has |
| | subsequently been used and validated in numerous chronic |
| | conditions. FACIT-Fatigue has been used to assess the impact of |
| | fatigue in a number of chronic autoimmune diseases. FACIT- |
| | Fatigue scores range from 0-52 (higher scores indicate less fatigue). |
| Outcome Measure Title | Absolute change from baseline in Living with Pulmonary |
| | Fibrosis (L-PF) total symptom score at Week 52. |
| | Outcome measure description: |
| | The Living with Idiopathic Pulmonary Fibrosis (L-PF) |
| | questionnaire was developed with input from patients with |
| | idiopathic pulmonary fibrosis (IPF) to assess symptoms and |
| | health-related quality of life (HRQOL). It consists of 44 items |
| | divided into two modules: Symptoms (23 items) and Impacts |
| | (21 items). The Symptoms module assesses shortness of breath, |
| | cough, and fatigue in the past 24 hours. The Impacts module |
| | assesses multiple aspects of HRQOL with a recall period of 1 |
| | week. Items in both modules have response options on a five- |
| | option numeric rating score with 'n' anchor of 0 “Not at all” to 4 |
| | “Extremely”. Overall scores range from 0 to 100, with higher |
| | numbers indicating a greater impairment (Swigris, et al. ERJ |
| | Open Res. 2021; 7(2):00145-2020). |
| Outcome Measure Title | Absolute change from baseline in quantitative interstitial lung |
| | disease in the whole lung (QILD-WL) at Week 52. |
| | Absolute change from baseline in quantitative measures of lung |
| | fibrosis in the whole lung (QLF-WL) at Week 52. |
| | Achieving ≥2% decrease in QILD-WL score at Week 52. |
| | Outcome measure description: |
| | QLF measures the extent of reticular patterns with architectural |
| | distortion (fibrosis) and the QILD score incorporates QLF, |
| | ground glass opacification and honeycombing features. |
| | Quantitative analysis is conducted using a computer-aided |
| | analysis tool for fibrotic patterns based on texture measures from |
| | high-resolution computed tomography HRCT (Kim, et al. |
| | Clinical and experimental rheumatology vol. 28, 5 Suppl 62 |
| | (2010): S26-35; and Kim, et al. European radiology vol. 21,12 |
| | (2011): 2455-65). |
| Outcome Measure Title | Achieving relative decline from baseline in FVC (mL) ≥5% at |
| | Week 52. |
| | Achieving relative decline from baseline in FVC (mL) ≥10% at |
| | Week 52. |
| Outcome Measure Title | Cumulative dose of corticosteroid over 52 weeks |
| Outcome Measure Title | Time to CTD progression |
| | Outcome measure description: |
| | CTD progression is defined as worsening of existing, or |
| | development of new extra-pulmonary disease manifestation(s) |
| | that require treatment escalation in accordance with local |
| | treatment guidelines. |
| Outcome Measure Title | Absolute change from baseline in Transition Dyspnea Index |
| | (TDI) at Week 52. |
| | Absolute change from baseline in Short form health survey |
| | 36-item version 2 (SF36-v2) at Week 52. |
| | Absolute change from baseline in Living with Pulmonary |
| | Fibrosis (L-PF) Impacts total score at Week 52. |
| | Absolute change from baseline in King's Brief Interstitial Lung |
| | Disease Questionnaire (K-BILD) at Week 52. |
| | Absolute change from baseline in physician global assessment |
| | (PhGA) at Week 52. |
| | Absolute change in Patient Global Impression of Change and |
| | Meaningfulness of Change (PGIC)-ILD at week 52. |
| | Outcome measure description: |
| | Baseline Dyspnea Index (BDI) and Transition Dyspnea Index |
| | (TDI): BDI and TDI assess dyspnea severity at baseline and its |
| | change over time. Each index includes 3 components: functional |
| | impairment, magnitude of task and magnitude of effort. For BDI, |
| | each component has 5 grades, from 0 (severe) to 4 (unimpaired), |
| | which are summed to calculate a score, ranging between 0 and 12. |
| | For TDI, each component has 7 grades, ranging from −3 |
| | (major deterioration) to +3 (major improvement), which are |
| | summed to calculate a score, ranging between −9 and +9. |
| | Short-Form Health Survey 36-item version 2 (SF-36v2) is a |
| | generic health survey that contains 36 questions covering eight |
| | domains of health. The SF-36v2 yields an eight-scale profile of |
| | functional health and well-being scores as well as physical and |
| | mental component summary scores. The lower the score the more |
| | the disability. |
| | The King's Brief Interstitial Lung Disease (K-BILD) |
| | questionnaire is a patient-reported outcome (PRO) measure |
| | specifically developed and validated for a range of ILDs. It was |
| | designed to measure the impact of ILD on the patient's wellbeing |
| | and daily life. K-BILD includes 15 items in 3 domains |
| | (Breathlessness and activity: 4 items, Psychological: 7 items, |
| | Chest symptoms: 3items) and a single item related to finance. |
| | Participants are to rate the impact of ILD disease on various |
| | aspects of their life during the past 2 weeks using 7-point rating |
| | scale (e.g., “All of the time”, “Most of the time”, “A good bit of |
| | the time”, “Some of the time”, “A little of the time”, “Hardly any |
| | of the time”, and “None of the time”). A total score and 3 domain |
| | scores are calculated ranging from 0-100 with greater scores |
| | denoting better quality of life (QoL). |
| | The PhGA is a score which enables the treating physician to rate |
| | the participants disease on a scale from 0 to 10, where higher |
| | score indicates greater severity. |
| | The PGIC-ILD contains two items, a global question asking |
| | participants to rate their overall change in ILD severity since first |
| | starting this study, and a yes/no question asking participants to |
| | indicate whether the change is meaningful from their perspective. |
| | Participants will complete PGIC-ILD using a 7-point verbal |
| | rating scale from “Very much worse”, “Moderately worse”, |
| | “Minimally worse”, “No change”, “Minimally improved”, |
| | “Moderately improved”, to “Very much improved”. Participants |
| | then will complete “Yes” or “No” regarding whether the change |
| | that they experienced was meaningful to them or not. |
| Outcome Measure Title | Absolute change from baseline in Diffusing Capacity of the lung |
| | for Carbon Monoxide (DLco) % predicted at Week 52. |
| | Outcome Measure Description: |
| | Single breath DLco will be carried out according to the ATS/ |
| | ERS guideline on DLco measurements (Graham, et al. 2017. Eur |
| | Respir J. 2017;49:1600016). DLco values will be adjusted for the |
| | most recent hemoglobin value. |
| Outcome Measure Title | Incidence of Adverse Events (Aes), Adverse Events of Special |
| | Interest (AESIs) and Serious Adverse Events (SAEs) up to |
| | Week 52. |
| | Incidence of respiratory related hospitalisations up to Week 52. |

### Inclusion and Exclusion Criteria:

| | |
|---|---|
| Inclusion | The Inclusion Criteria for this study comprise: |
| Criteria | 1. Participant is 18 years of age inclusive, or older at the |
| | time of signing the informed consent. |
| | 2. Documented diagnosis of rheumatoid arthritis (RA), |
| | systemic lupus erythematosus (SLE), idiopathic |
| | inflammatory myopathy (IIM; including polymyositis, |
| | dermatomyositis, anti-synthetase syndrome), Sjogren's |
| | syndrome (pSS), or mixed connective tissue disease |
| | (MCTD) in accordance with internationally recognized |
| | classification criteria. Patients with overlap syndrome |
| | (including SSc overlap) are permitted if their primary |
| | diagnosis is RA, SLE, IIM, pSS, or MCTD. |
| | 3. Diagnosis of ILD on HRCT with disease extent of ≥10% |
| | of the whole lung (WL-ILD), as confirmed by central |
| | reader at screening. The 10% extent of ILD includes the |
| | combined assessment of reticulation/fibrosis and ground |
| | glass opacity). |
| | 4. Evidence of ILD progression in the previous 24 months. |
| | 5. Must be currently receiving stable standard therapy to |
| | manage ILD and/or underlying CTD, or to have failed or |
| | failed to tolerate first line standard therapy. |
| | 6. Participant is capable and willing to self-administer the |
| | study medication or has a caregiver who is capable and |
| | willing to administer the study medication throughout |
| | the study. |
| | 7. Female participants: A female participant is eligible to |
| | participate if she is not pregnant or breastfeeding, and |
| | one of the following conditions applies: |
| | a. Is a woman of non-childbearing potential |
| | (WONCBP); OR |
| | b. Is a woman of childbearing potential (WOCBP) |
| | and using a contraceptive method that is highly |
| | effective, with a failure rate of <1%. |
| | 8. Capable of giving signed informed consent. |
| Exclusion | The Exclusion Criteria for this study comprise: |
| Criteria | 1. Diagnosis of ILD other than CTD-ILD. |
| | 2. Primary diagnosis of Systemic Sclerosis (SSc). |
| | 3. Participants with rapidly progressive disease (absolute |
| | drop of 10% or more of FVC between screening and |
| | baseline visit and/or recent pulmonary hospitalisation). |
| | 4. FVC ≤45% of predicted, or a Dlco (corrected for |
| | hemoglobin) ≤40% of predicted or requiring |
| | supplemental oxygen at screening. |
| | 5. History or presence of diffuse alveolar haemorrhage |
| | (DAH) or other confounding pulmonary disease, signs, |
| | or symptoms. |
| | 6. Pulmonary arterial hypertension requiring therapy, as |
| | determined by the investigator at, or prior to first day of |
| | dosing (Day 1). |
| | 7. Dependence on continuous oxygen supplementation. |
| | 8. History or presence of cardiovascular, respiratory, |
| | hepatic, renal, gastrointestinal, endocrine, hematologic, |
| | or neurological disorders capable of significantly |
| | altering the absorption, metabolism, or elimination of |
| | drugs; constituting a risk when taking the study |
| | intervention or interfering with the interpretation of data. |
| | 9. Obstructive pulmonary disease (pre-bronchodilator |
| | forced expiratory volume in 1 second (FEV1)/FVC <0.7). |
| | 10. Significant emphysema on screening HRCT (extent of |
| | emphysema exceeds extent of ILD). |
| | 11. Confirmed progressive Multifocal Leukoencephalopathy |
| | (PML) or unexplained new-onset or deteriorating |
| | neurologic signs and symptoms. |

_{SEQUENCES}

SEQ ID NO: 1: belimumab CDRH1
GGTFNNNAIN
SEQ ID NO: 2: belimumab CDRH2
GIIPMFGTAKYSQNFQG
SEQ ID NO: 3: belimumab CDRH3
SRDLLLFPHHALSP
SEQ ID NO: 4: belimumab CDRL1
QGDSLRSYYAS
SEQ ID NO: 5: belimumab CDRL2
GKNNRPS
SEQ ID NO: 6: belimumab CDRL3
SSRDSSGNHWV
SEQ ID NO: 7: belimumab VH
QVQLQQSGAEVKKPGSSVRVSCKASGGTFNNNAINWVRQAPGQGLEWMG
GIIPMFGTAKYSQNFQGRVAITADESTGTASMELSSLRSEDTAVYYCAR
SRDLLLFPHHALSPWGRGTMVTVSS
SEQ ID NO: 8: belimumab VL
SSELTQDPAVSVALGQTVRVTCQGDSLRSYYASWYQQKPGQAPVLVIYG
KNNRPSGIPDRFSGSSSGNTASLTITGAQAEDEADYYCSSRDSSGNHWV
FGGGTELTVLG
SEQ ID NO: 9: belimumab heavy chain
QVQLQQSGAEVKKPGSSVRVSCKASGGTFNNNAINWVRQAPGQGLEWMG
GIIPMFGTAKYSQNFQGRVAITADESTGTASMELSSLRSEDTAVYYCAR
SRDLLLFPHHALSPWGRGTMVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK
SEQ ID NO: 10: belimumab light chain
SSELTQDPAVSVALGQTVRVTCQGDSLRSYYASWYQQKPGQAPVLVIYG
KNNRPSGIPDRFSGSSSGNTASLTITGAQAEDEADYYCSSRDSSGNHWV
FGGGTELTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECS

1-40. (canceled)

41. A method for treatment of connective tissue disease-associated interstitial lung disease using an anti-BlyS antibody in a human subject in need thereof, wherein the connective tissue disease-associated interstitial lung disease comprises systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease, and wherein the connective tissue disease-associated interstitial lung disease does not comprise systemic lupus erythematosus-associated interstitial lung disease.

42. The method according to claim 41, wherein the interstitial lung disease does not comprise usual interstitial pneumonia.

43. The method according to claim 41, wherein the interstitial lung disease comprises usual interstitial pneumonia.

44. The method according to claim 43, wherein the connective tissue disease-associated interstitial lung disease is rheumatoid arthritis-associated interstitial lung disease.

45. The method according to claim 41, wherein the interstitial lung disease comprises nonspecific interstitial pneumonia, organising pneumonia, and/or lymphocytic interstitial pneumonia.

46. The method according to claim 41, wherein the connective tissue disease-associated interstitial lung disease is systemic sclerosis-associated interstitial lung disease, rheumatoid arthritis-associated interstitial lung disease, Sjögren's syndrome-associated interstitial lung disease, an idiopathic inflammatory myopathy-associated interstitial lung disease, and/or mixed connective tissue disease-associated interstitial lung disease, and wherein the interstitial lung disease comprises nonspecific interstitial pneumonia, or
wherein the connective tissue disease-associated interstitial lung disease is Sjögren's syndrome-associated interstitial lung disease, and wherein the interstitial lung disease comprises lymphocytic interstitial pneumonia, or
wherein the connective tissue disease-associated interstitial lung disease is an idiopathic inflammatory myopathy-associated interstitial lung disease, and wherein the interstitial lung disease comprises organising pneumonia.

47. The method according to claim 46, wherein the connective tissue disease-associated interstitial lung disease is an idiopathic inflammatory myopathy-associated interstitial lung disease, and wherein the idiopathic inflammatory myopathy comprises polymyositis, dermatomyositis, or anti-synthetase syndrome.

48. The method according to claim 41, wherein the treatment is characterised by:
(A) a reduction in a disease progression, optionally, wherein the disease progression is connective tissue disease progression and/or interstitial lung disease progression, and/or
(B) an improvement in one or more measures relative to baseline in the human subject, wherein the one or more measures are selected from:
(i) forced vital capacity;
(ii) diffusing capacity of the lung for carbon monoxide;
(iii) connective tissue disease-associated interstitial lung disease symptom severity, optionally, wherein the improvement in connective tissue disease-associated interstitial lung disease symptom severity comprises a reduction in fatigue, a reduction in dyspnea, and/or a reduction in cough relative to baseline; and/or
(iv) quality of life, and/or
(C) a reduction in one or more measures relative to baseline in the human subject, wherein the one or more measures are selected from:
(v) extent of interstitial lung disease; and/or
(vi) corticosteroid use.

49. The method according to claim 48, part (A), wherein the disease progression is interstitial lung disease progression, and wherein the interstitial lung disease progression is characterised by a reduction in forced vital capacity, optionally, wherein the interstitial lung disease progression is further characterised by:
(i) a reduction in forced vital capacity of 55% relative to baseline, optionally, a reduction in forced vital capacity of 100% relative to baseline; or
(ii) a reduction in forced vital capacity of 55% to <10% relative to baseline and a reduction in diffusing capacity of the lung for carbon monoxide of ≥15% relative to baseline, or wherein the disease progression is interstitial lung disease progression, and wherein the interstitial lung disease progression is characterised by two or more of the following (a) to (c) occurring within a 12 month time period:
(a) a worsening of respiratory symptoms;
(b) a physiological evidence of interstitial lung disease progression, wherein the physiological evidence of interstitial lung disease progression comprises:
(i) an absolute reduction in forced vital capacity of 55% predicted; and/or
(ii) an absolute reduction in diffusing capacity of the lung for carbon monoxide of ≥10% predicted; and/or
(c) a radiological evidence of interstitial lung disease progression.

50. The method according to claim 41, for use in the treatment of systemic sclerosis-associated interstitial lung disease in the human subject.

51. The method according to claim 50, in the treatment of:
(A) diffuse cutaneous systemic sclerosis-associated interstitial lung disease, optionally, for use in the treatment of early diffuse cutaneous systemic sclerosis-associated interstitial lung disease, or
(B) limited cutaneous systemic sclerosis-associated interstitial lung disease, optionally, for use in the treatment of early limited cutaneous systemic sclerosis-associated interstitial lung disease.

52. The method according to claim 50, wherein the treatment is characterised by a reduction in disease progression, optionally, wherein the disease progression is characterised by a reduction in forced vital capacity.

53. The method according to claim 52, wherein the disease progression is further characterised by:
(i) a reduction in forced vital capacity of 55% relative to baseline, optionally, a reduction in forced vital capacity of ≥10% relative to baseline; or
(ii) a reduction in forced vital capacity of 55% to <10% relative to baseline and a reduction in diffusing capacity of the lung for carbon monoxide of ≥15% relative to baseline, or
wherein the disease progression is further characterised by an increase in modified Rodnan skin score of ≥20% relative to baseline.

54. The method according to claim 50, wherein the treatment is characterised by:
(a) an improvement in forced vital capacity relative to baseline; and/or
(b) a reduction in modified Rodnan skin score of ≥20% relative to baseline.

55. The method according to claim 50, wherein a reduction in one or more measures relative to baseline is achieved in the human subject, wherein the one or more measures are selected from:
(i) Fatigue, optionally, wherein the fatigue is as assessed by the functional assessment of chronic illness therapy-fatigue score compared to baseline;
(ii) Health Assessment Questionnaire Disability Index score; and/or
(iii) Scleroderma skin patient-reported outcome score, and/or
wherein an increase in one or more measures relative to baseline is achieved in the human subject, wherein the one or more measures are selected from:
(i) Transition dyspnoea index score; and/or
(ii) Medical outcome study 36-item short form score.

56. The method according to claim 50, wherein the human subject is characterised by one or more of:
(i) Presence of thickened skin with a modified Rodnan Skin Score ≥10 points, optionally, ≥15 points;
(ii) Anti-topoisomerase I (anti-Scl70) autoantibody positive;
(iii) Anti-RNA polymerase autoantibody positive;
(iv) Anti-centromere autoantibody negative; and
(v) Disease duration ≤7 years, and/or
wherein the human subject is anti-topoisomerase I (anti-Scl70) autoantibody positive.

57. The method according to claim 50, wherein the human subject has a disease duration of ≥2 years and one or more of:
(i) Reduction in forced vital capacity;
(ii) Reduction in diffusing capacity of the lung for carbon monoxide;
(iii) Anti-topoisomerase I (anti-Scl70) autoantibody positive; and/or
(iv) Increased extent of interstitial lung disease on high-resolution computed tomography.

58. The method according to claim 41, wherein the anti-BlyS antibody comprises:
(A) CDRH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6 or variants thereof, and/or
(B) CRDH1 of SEQ ID NO: 1; CDRH2 of SEQ ID NO: 2; CDRH3 of SEQ ID NO: 3; CDRL1 of SEQ ID NO: 4; CDRL2 of SEQ ID NO: 5; and CDRL3 of SEQ ID NO: 6, and/or
(C) a variable heavy chain sequence of SEQ ID NO: 7, or a sequence which is at least 90% identical thereto, and a variable light chain sequence of SEQ ID NO: 8, or a sequence which is at least 90% identical thereto, and/or
(D) a variable heavy chain sequence of SEQ ID NO: 7 and a variable light chain sequence of SEQ ID NO: 8.

59. The method according to claim 41, wherein the anti-BlyS antibody is belimumab.

60. The method according to claim 41, wherein the anti-BlyS antibody is administered subcutaneously, optionally, wherein the anti-BlyS antibody is administered to the human subject at a unit dose of 200 mg per week.

61. The method according to claim 41, wherein the anti-BlyS antibody is co-administered with an additional immunosuppressive agent, optionally, wherein the additional immunosuppressive agent is selected from methotrexate, mycophenolate mofetil, mycophenolate sodium, azathioprine, a corticosteroid, a calcineurin inhibitor, a pyrimidine synthesis inhibitor, and/or an anti-malarial agent.


SEQUENCE LISTING (10 sequences)

SEQ ID NO: 1 - protein, 10 aa, synthetic construct
GGTFNNNAIN

SEQ ID NO: 2 - protein, 17 aa, synthetic construct
GIIPMFGTAKYSQNFQG

SEQ ID NO: 3 - protein, 14 aa, synthetic construct
SRDLLLFPHHALSP

SEQ ID NO: 4 - protein, 11 aa, synthetic construct
QGDSLRSYYAS

SEQ ID NO: 5 - protein, 7 aa, synthetic construct
GKNNRPS

SEQ ID NO: 6 - protein, 11 aa, synthetic construct
SSRDSSGNHWV

SEQ ID NO: 7 - protein, 123 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSS

SEQ ID NO: 8 - protein, 109 aa, synthetic construct
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 9 - protein, 453 aa, synthetic construct
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS